(19) United States
(12) Patent Application Publication
(10) Pub. No.: US 2004/0009498 A1
(43) Pub. Date: Jan. 15, 2004

(54) Chimeric antigen binding molecules and methods for making and using them

(75) Inventor: Jay M. Short, Rancho Santa Fe, CA (US)

Correspondence Address: FISH & RICHARDSON, PC, 12390 EL CAMINO REAL, SAN DIEGO, CA, 92130-2081, US

(73) Assignee: Diversa Corporation, San Diego, CA

(21) Appl. No.: 10/345,826

(22) Filed: Jan. 14, 2003

Related U.S. Application Data
(60) Provisional application No. 60/348,761, filed on Jan. 14, 2002.

(51) Int. Cl. C12Q 1/68; C07H 21/04; C12P 19/34

(52) U.S. Cl. 435/6; 435/91.2; 536/23.2

(57) ABSTRACT

The invention provides methods for identifying and purifying double-stranded polynucleotides lacking base pair mismatches, insertion/deletion loops and/or nucleotide gaps. The invention provides libraries of nucleic acid building blocks and methods for generating any nucleic acid sequence, including synthetic genes, antisense constructs and polypeptide coding sequences. The invention provides chimeric antigen binding molecules and the nucleic acids that encode them.

### RELATED APPLICATIONS

This application claims the benefit of priority under 35 U.S.C. § 119(e) of U.S. Provisional Application No. 60/348,761, filed Jan. 14, 2002. The aforementioned application is explicitly incorporated herein by reference in its entirety and for all purposes.

### TECHNICAL FIELD

The present invention is generally directed to the fields of genetic and protein engineering and molecular biology. In particular, the invention provides methods for identifying and purifying double-stranded polynucleotides lacking base pair mismatches, insertion/deletion loops and nucleotide gaps.

The present invention is generally directed to the fields of protein and genetic engineering and molecular biology. In one aspect, the invention is directed to libraries of oligonucleotides and methods for generating any nucleic acid sequence, including synthetic genes, antisense constructs and polypeptide coding sequences. In one aspect, the libraries of the invention comprise oligonucleotides comprising restriction endonuclease restriction sites, e.g., Type-IIS restriction endonuclease restriction sites, wherein the restriction endonuclease cuts at a fixed position outside of the recognition sequence to generate a single stranded overhang. The polynucleotide construction methods comprise use of libraries of pre-made multicodon (e.g., dicodon) oligonucleotide building blocks and Type-IIS restriction endonucleases.

In one aspect, the invention is directed to methods for generating sets, or libraries, of nucleic acids encoding chimeric antigen binding molecules, including, e.g., antibodies and related molecules, such as antigen binding sites and domains and other antigen binding fragments, including single and double stranded antibodies. This invention provides methods for generating new or variant chimeric antigen binding polypeptides, e.g., antigen binding sites, antibodies and specific domains or fragments of antibodies (e.g., Fab or Fc domains) by altering the nucleic acids that encode them by, e.g., saturation mutagenesis, an optimized directed evolution system, synthetic ligation reassembly, or a combination thereof.

The invention also provides libraries of chimeric antigen binding polypeptides encoded by the nucleic acid libraries of the invention and generated by the methods of the invention. These antigen binding polypeptides can be analyzed using any liquid or solid state screening method, e.g., phage display, ribosome display, using capillary array platforms, and the like. The polypeptides generated by the methods of the invention can be used in vitro, e.g., to isolate or identify antigens or in vivo, e.g., to treat or diagnose various diseases and conditions, to modulate, stimulate or attenuate an immune response. The invention also is directed to the generation of chimeric immunoglobulins for administering passive immunity and nucleic acids encoding these chimeric antigen binding molecules for genetic vaccines.

### BACKGROUND

Synthetic oligonucleotides are commonly used to construct nucleic acids, including polypeptide coding sequences and gene constructs. However, even the best oligonucleotide synthesizer has a 1% to 5% error rate. These errors can result in improper base pair sequences, which can lead to generation of an erroneous protein sequences. These errors can also result in sequences that cannot be properly transcribed or untranslated, including, e.g., premature stop codons. To detect these errors, the oligonucleotides or the sequences generated using the oligonucleotides are sequenced. However, sequencing to detect errors in nucleic acid synthetic techniques is time consuming and expensive.

Engineering genes, polypeptide coding sequences and other polynucleotide molecules can be impeded by the need to isolate, synthesize or handle a parental, or template, DNA sequence. For example, it may be necessary to alter codon usage for optimal expression in a cell host, requiring manipulation of the polynucleotide sequence. Frequently is it desirable or necessary to add and/or remove restriction sites to an isolated, cloned or amplified polynucleotide to facilitate manipulation of the sequence, requiring further modification of the molecule. All of these manipulations introduce labor costs and are potential sources of sequence and cloning errors.

The best quality oligonucleotide synthesis systems available still contain up to 1% of (n−1) and (n−2) contaminations leading to a high error rate in the nucleic acid sequences (e.g. genes, gene pathways, or regulatory motifs) built. These errors can manifest themselves as frame shifts or as stop codon, resulting in truncated proteins if the engineered gene is expressed. Sometimes, more than 20 clones have to be sequenced and errors corrected (e.g., by site directed mutagenesis) to get the desired nucleotide sequence for a single gene or coding sequence. In the case of chimeric polynucleotide libraries sequencing and correcting all errors is not an option and oligo-based sequence errors decrease cloning and screening efficiency significantly.

Antigen binding polypeptides, such as antibodies, are increasingly used in a variety of therapeutic applications. For example, in immunotherapy, antibodies are used to directly kill target cells, such as cancer cells. They can be administered to generate passive immunity. Antigen binding polypeptides are also used as carriers to deliver cytotoxic or imaging reagents. Monoclonal antibodies (mAbs) approved for cancer therapy are now in Phase II and III trials. Certain anti-idiotypic antibodies that bind to the antigen-combining sites of antibodies can effectively mimic the three-dimensional structures and functions of the external antigens and can be used as surrogate antigens for active specific immunotherapy. Bi-specific antibodies combine immune cell activation with tumor cell recognition; thus, tumor cells or cells expressing tumor specific antigens (e.g., tumor vasculature) are killed by pre-defined effector cells. Antibodies can be administered to increase or decrease the levels of cytokines or hormones by direct binding or by stimulating or inhibiting secretory cells. Accordingly, increasing the affinity or avidity of an antibody to a desired antigen, such as a cancer-specific antigen, would result in greater specificity of the antibody to its target, resulting in a variety of therapeutic benefits, such as needing to administer less antibody-containing pharmaceutical.

### SUMMARY

Methods for Purifying and Identifying Double-stranded Nucleic Acids Lacking Base Pair Mismatches, Insertion/deletion Loops or Nucleotide Gaps

The invention provides methods for identifying and purifying double-stranded polynucleotides lacking nucleotide gaps, base pair mismatches and insertion/deletion loops. In one aspect, the invention provides methods for purifying double-stranded polynucleotides lacking base pair mismatches, insertion/deletion loops and/or nucleotide gaps comprising the following steps: (a) providing a plurality of polypeptides that specifically bind to a base pair mismatch, an insertion/deletion loop and/or a nucleotide gap or gaps within a double stranded polynucleotide; (b) providing a sample comprising a plurality of double-stranded polynucleotides; (c) contacting the double-stranded polynucleotides of step (b) with the polypeptides of step (a) under conditions wherein a polypeptide of step (a) can specifically bind to a base pair mismatch, an insertion/deletion loop and/or a nucleotide gap or gaps in a double stranded polynucleotide of step (b); and (d) separating the double-stranded polynucleotides lacking a specifically bound polypeptide of step (a) from the double-stranded polynucleotides to which a polypeptide of step (a) has specifically bound, thereby purifying double-stranded polynucleotides lacking base pair mismatches, insertion/deletion loops and/or nucleotide gaps. In one aspect, the double-stranded polynucleotide comprises a double-stranded oligonucleotide. In one aspect, the double-stranded polynucleotide consists of a double-stranded oligonucleotide.

In alternative aspects, the double-stranded polynucleotide is between about 3 and about 300 base pairs in length; between 10 and about 200 base pairs in length; and, between 50 and about 150 base pairs in length. In alternative aspects, the gaps in the double-stranded polynucleotide are between about 1 and 30, about 2 and 20, about 3 and 15, about 4 and 12 and about 5 and 10 nucleotides in length.

In alternative aspects, the the base pair mismatch comprises a C:T mismatch, a G:A mismatch, a C:A mismatch or a G:U/T mismatch.

In one aspect, the polypeptide that specifically binds to a base pair mismatch, an insertion/deletion loop and/or a nucleotide gap or gaps in a double stranded polynucleotide comprises a DNA repair enzyme. In alternative aspects, the DNA repair enzyme is a bacterial DNA repair enzyme, a MutS DNA repair enzyme, a Taq MutS DNA repair enzyme, an Fpg DNA repair enzyme, a MutY DNA repair enzyme, a hexA DNA mismatch repair enzyme, a Vsr mismatch repair enzyme, a mammalian DNA repair enzyme and natural or synthetic variations and isozymes thereof. In one aspect, the DNA repair enzyme is a DNA glycosylase that initiates base-excision repair of G:U/T mismatches. The DNA glycosylase can comprise a bacterial mismatch-specific uracil-DNA glycosylase (MUG) DNA repair enzyme or a eukaryotic thymine-DNA glycosylase (TDG) enzyme.

In one aspect, the separating of the double-stranded polynucleotides lacking a specifically bound polypeptide of step (a) from the double-stranded polynucleotides to which a polypeptide of step (a) has specifically bound of step (d) comprises use of an immunoaffinity column, wherein the column comprises immobilized antibodies capable of specifically binding to the specifically bound polypeptide or an epitope bound to the specifically bound polypeptide, and the sample is passed through the immunoaffinity column under conditions wherein the immobilized antibodies are capable of specifically binding to the specifically bound polypeptide or the epitope bound to the specifically bound polypeptide.

In one aspect, the separating of the double-stranded polynucleotides lacking a specifically bound polypeptide of step (a) from the double-stranded polynucleotides to which a polypeptide of step (a) has specifically bound of step (d) comprises use of an antibody, wherein the antibody is capable of specifically binding to the specifically bound polypeptide or an epitope bound to the specifically bound polypeptide and the antibody is contacted with the specifically bound polypeptide under conditions wherein the antibodies are capable of specifically binding to the specifically bound polypeptide or an epitope bound to the specifically bound polypeptide. The antibody can be an immobilized antibody. The antibody can be immobilized onto a bead or a magnetized particle or a magnetized bead.

In one aspect, the separating of the double-stranded polynucleotides lacking a specifically bound polypeptide of step (a) from the double-stranded polynucleotides to which a polypeptide of step (a) has specifically bound of step (d) comprises use of an affinity column, wherein the column comprises immobilized binding molecules capable of specifically binding to a tag linked to the specifically bound polypeptide and the sample is passed through the affinity column under conditions wherein the immobilized antibodies are capable of specifically binding to the tag linked to the specifically bound polypeptide. The immobilized binding molecules can comprise an avidin or a natural or synthetic variation or homologue thereof and the tag linked to the specifically bound polypeptide can comprise a biotin or a natural or synthetic variation or homologue thereof.

In one aspect, the separating of the double-stranded polynucleotides lacking a specifically bound polypeptide of step (a) from the double-stranded polynucleotides to which a polypeptide of step (a) has specifically bound of step (d) comprises use of a size exclusion column, such as a spin column. Alternatively, the separating can comprise use of a size exclusion gel, such as an agarose gel.

In one aspect, the double-stranded polynucleotide comprises a polypeptide coding sequence. The polypeptide coding sequence can comprise a fusion protein coding sequence. The fusion protein can comprise a polypeptide of interest upstream of an intein, wherein the intein comprises a polypeptide. The intein polypeptide can comprise an enzyme, such as one used to identify vector or insert positive clones, such as Lac Z. The intein polypeptide can comprise an antibody or a ligand. In one aspect, the intein polypeptide comprises a polypeptide selectable marker, such as an antibiotic. The antibiotic can comprise a kanamycin, a penicillin or a hygromycin.

The invention provides a method for assembling double-stranded oligonucleotides to generate a polynucleotide lacking base pair mismatches, insertion/deletion loops and/or nucleotide gaps comprising the following steps: (a) providing a plurality of polypeptides that specifically bind to a base pair mismatch, an insertion/deletion loop and/or a nucleotide gap or gaps in a double stranded polynucleotide; (b) providing a sample comprising a plurality of double-stranded oligonucleotides; (c) contacting the double-stranded oligonucleotides of step (b) with the polypeptides of step (a) under conditions wherein a polypeptide of step (a) can specifically bind to a base pair mismatch, an insertion/deletion loop and/or a nucleotide gap or gaps in a double stranded oligonucleotide of step (b); (d) separating the double-stranded oligonucleotides lacking a specifically bound polypeptide of step (a) from the double-stranded oligonucleotides to which a polypeptide of step (a) has specifically bound, thereby purifying double-stranded oligonucleotides lacking base pair mismatches, insertion/deletion loops and/or a nucleotide gap or gaps; and (e) joining together the purified double-stranded oligonucleotides lacking base pair mismatches and insertion/deletion loops, thereby generating a polynucleotide lacking base pair mismatches, insertion/deletion loops and/or nucleotide gaps.

In one aspect, the double-stranded oligonucleotides comprise libraries of oligonucleotides, e.g., the libraries of the invention comprising oligonucleotides comprising multicodons. For example, the double-stranded oligonucleotides can comprise libraries of oligonucleotides comprising multicodon, e.g., dicodon, building blocks. In one aspect, the library comprises a plurality of double-stranded oligonucleotide members, wherein each oligonucleotide member comprises two or more codons in tandem (e.g., a dicodon) and a Type-IIS restriction endonuclease recognition sequence flanking the 5′ and the 3′ end of the multicodon (e.g., dicodon, tricodon, tetracodon, and the like).

The invention provides a method for generating a polynucleotide lacking base pair mismatches, insertion/deletion loops and/or nucleotide gaps comprising the following steps: (a) providing a plurality of polypeptides that specifically bind to a base pair mismatch, an insertion/deletion loop and/or a nucleotide gap or gaps in a double stranded polynucleotide; (b) providing a sample comprising a plurality of double-stranded oligonucleotides; (c) joining together the double-stranded oligonucleotides of step (b) to generate a double-stranded polynucleotide; (d) contacting the double-stranded polynucleotide of step (c) with the polypeptides of step (a) under conditions wherein a polypeptide of step (a) can specifically bind to a base pair mismatch, an insertion/deletion loop and/or a nucleotide gap or gaps in a double stranded polynucleotide of step (c); and (e) separating the double-stranded polynucleotides lacking a specifically bound polypeptide of step (a) from the double-stranded polynucleotides to which a polypeptide of step (a) has specifically bound, thereby purifying double-stranded polynucleotides lacking base pair mismatches, insertion/deletion loops and/or nucleotide gaps. In one aspect, the double-stranded oligonucleotides comprise a library of oligonucleotides multicodon building blocks, the library comprising a plurality of double-stranded oligonucleotide members, wherein each oligonucleotide member comprises at least two codons in tandem and a Type-IIS restriction endonuclease recognition sequence flanking the 5′ and the 3′ end of the multicodon.

In one aspect, the method further comprises providing a set of 61 immobilized starter oligonucleotides, one oligonucleotide for each possible amino acid coding triplet, wherein the oligonucleotides are immobilized on a substrate and have a single-stranded overhang corresponding to a single-stranded overhang generated by a Type-IIS restriction endonuclease, or, the oligonucleotides comprise a Type-IIS restriction endonuclease recognition site distal to the substrate and a single-stranded overhang is generated by digestion with a Type-IIS restriction endonuclease: digesting a second oligonucleotide member from the library of step (a) with a Type-IIS restriction endonuclease to generate a single-stranded overhang; and contacting the digested second oligonucleotide member to the immobilized first oligonucleotide member under conditions wherein complementary single-stranded base overhangs of the first and the second oligonucleotides can pair, and, ligating the second oligonucleotide to the first oligonucleotide, thereby generating a double-stranded polynucleotide.

The invention provides a method for generating a base pair mismatch-free, insertion/deletion loop-free and/or gap-free double-stranded polypeptide coding sequence comprising the following steps: (a) providing a plurality of polypeptides that specifically bind to a base pair mismatch, an insertion/deletion loop and/or a nucleotide gap or gaps within a double stranded polynucleotide; (b) providing a sample comprising a plurality of double-stranded polynucleotides encoding a fusion protein, wherein the fusion protein coding sequence comprises a coding sequence for a polypeptide of interest upstream of and in frame with a coding sequence for a marker or a selection polypeptide; (c) contacting the double-stranded polynucleotides of step (b) with the polypeptides of step (a) under conditions wherein a polypeptide of step (a) can specifically bind to a base pair mismatch, an insertion/deletion loop and/or a nucleotide gap or gaps in a double stranded polynucleotide of step (b);

(d) separating the double-stranded polynucleotides lacking a specifically bound polypeptide of step (a) from the double-stranded polynucleotides to which a polypeptide of step (a) has specifically bound, thereby purifying double-stranded polynucleotides lacking base pair mismatches, insertion/deletion loops and/or a nucleotide gap or gaps; (e) expressing the purified double-stranded polynucleotides and selecting the polynucleotides expressing the selection marker polypeptide, thereby generating a base pair mismatch-free, insertion/deletion loop-free and/or gap-free double-stranded polypeptide coding sequence.

In one aspect, the marker or selection polypeptide comprises a self-splicing intein, and the method further comprises the self-splicing out of the intein marker or selection polypeptide from the upstream polypeptide of interest. The marker or selection polypeptide can comprise an enzyme, such as a enzyme used to identity insert or vector-positive clones, such as a LacZ enzyme. The marker or selection polypeptide can also comprise an antibiotic, such as a kanamycin, a penicillin or a hygromycin.

In alternative aspects of the invention, the methods generate a sample or “batch” of purified oligonucleotides and/or polynucleotides that are 90%, 95%, 96%, 97%, 98%, 99%, 99.5% and 100% or completely free of base pair mismatches, insertion/deletion loops and/or a nucleotide gap or gaps.

The nucleic acids manipulated or altered by any means, including random or stochastic methods, or, non-stochastic, or “directed evolution,” can be “purified” or “processed” by the methods of the invention, e.g., the methods of the invention can be used to generate a sample or “batch” of double-stranded oligonucleotides and/or polynucleotides that are 90%, 95%, 96%, 97%, 98%, 99%, 99.5% and 100% or completely free of base pair mismatches, insertion/deletion loops and/or a nucleotide gap or gaps, wherein the nucleic acids (e.g., oligos, polynucleotides, genes, and the like) have been manipulated by stochastic methods, or, non-stochastic, or “directed evolution.” For example, the methods of the invention can be used to “purify” or “process” nucleic acids manipulated by saturation mutagenesis, an optimized directed evolution system, synthetic ligation reassembly, or a combination thereof, as described herein. The methods of the invention can be used to “purify” or “process” nucleic acids manipulated by a method comprising gene site saturated mutagenesis (GSSM). The methods of the invention can be used to “purify” or “process” nucleic acids manipulated by gene site saturated mutagenesis (GSSM), step-wise nucleic acid reassembly, error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, synthetic ligation reassembly (SLR) or a combination thereof. The methods of the invention can be used to “purify” or “process” nucleic acids manipulated by recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation or a combination thereof.

In one aspect, method of the invention comprises purifying a double-stranded nucleic acid comprising a synthetic, a naturally isolated, or a recombinantly generated nucleic acid (a polynucleotide or an oligonucleotide). The synthetic polynucleotide can be identical to a parental or a natural sequence. In one aspect, the polynucleotide comprises a gene, a chromosome. In one aspect, the gene further comprises a pathway. In one aspect, the gene comprises a regulatory sequence. In one aspect, the polynucleotide comprises a promoter or an enhancer or a polypeptide coding sequence. The polypeptide can be an enzyme, an antibody, a receptor, a neuropeptide, a chemokine, a hormone, a signal sequence, or a structural gene. In one aspect, the polynucleotide comprises non-coding sequence.

In one aspect, a polynucleotide purified by a method of the invention comprises a DNA (e.g., a gene or coding sequence), an RNA (e.g., an iRNA, an rRNA, a tRNA or an mRNA) or a combination thereof. For example, the methods of the invention can be used to generate a sample or “batch” of double-stranded DNA or RNA that are 90%, 95%, 96%, 97%, 98%, 99%, 99.5% and 100% or completely free of base pair mismatches, insertion/deletion loops and/or a nucleotide gap or gaps. In one aspect, the double-stranded polynucleotide comprises an iRNA. The double-stranded polynucleotide can comprise a DNA, e.g., a gene. In one aspect, the DNA comprises a chromosome.

Compositions and Methods for Making Polynucleotides by Assembly of Codon Building Blocks

The invention provides methods and compositions for making nucleic acids by iterative assembly of oligonucleotide building blocks. In one aspect, the invention provides libraries of oligonucleotides comprising multicodon (e.g., dicodon, tricodon) building blocks. In one aspect, the library comprises a plurality of double-stranded oligonucleotide members, wherein each oligonucleotide member comprises two or more codons in tandem (e.g., a dicodon) and a Type-IIS restriction endonuclease recognition sequence flanking the 5′ and the 3′ end of the multicodon (e.g., dicodon, tricodon, tetracodon, and the like).

In different aspects, this invention provides that the building blocks can be X-mers (where can be any integer from 3 to one billion). In other aspects, six-mers can be used that are not dicodons prior to assembly with other building blocks (because they are frame-shifted), but that can become codons after assembly with other building blocks. In other aspects, the intended product is not a coding sequence (but may be, e.g. a promoter, an enhancer, or any other regulatory motif), so the building blocks do not need to function as codons either before or after assembly with other building blocks. In other aspects, the assembly product can be, e.g., operons, gene pathways, chromosomes, or genomes. Thus, the term “codon” includes all nucleic acid sequences, including sequences that code for “non-coding” sequences such as regulatory motifs (e.g., promoters, enhancers), operons, structural sequences (e.g., telomeres) and the like.

In one aspect, the library comprises oligonucleotide members comprising all possible codon combinations, e.g., all possible dimer (dicodon) combinations, tricodon combinations, tetracodon combinations, and the like. In one aspect, the library of the invention can comprise oligonucleotide members comprising 4096 different possible codon dimer (dicodon) combinations (proteins are synthesized according to base triplets (codons) in a given DNA sequence; there are 61 different triplets coding for 20 different amino acids). The library can be of any size and can include anywhere from one to 4096 different members, e.g., the library can comprise about 50, 100, 150, 200, 250, 300, 350, 400, 450, 500, 600, 700, 800, 900, 1000, 2000, 3000, 4000 or more different members. In one aspect, none of the codons are stop codons.

In one aspect, the Type-IIS restriction endonuclease recognition sequence at the 5′ end of the dicodon differs from the Type-IIS restriction endonuclease recognition sequence at the 3′ end of the dicodon. The Type-IIS restriction endonuclease recognition sequence can be specific for a restriction endonuclease that, upon digestion of the oligonucleotide library member, generates a base overhang, including a one base single-stranded overhang, a two base single-stranded overhang, a three base single-stranded overhang, a four base single-stranded overhang, and the like. The restriction endonuclease can comprise a SapI restriction endonuclease or an isochizomer thereof, or, an EarI restriction endonuclease or an isochizomer thereof. In one aspect, the Type-IIS restriction endonuclease recognition sequence is specific for a restriction endonuclease that, upon digestion of the oligonucleotide library member, generates a two base single-stranded overhang. The restriction endonuclease can be a BseRI, a BsgI or a BpmI restriction endonuclease. In one aspect, the Type-IIS restriction endonuclease recognition sequence is specific for a restriction endonuclease that, upon digestion of the oligonucleotide library member, generates a one base single-stranded overhang. The restriction endonuclease can be an N.AlwI or an N.BstNBI restriction endonuclease.

In one aspect, the Type-IIS restriction endonuclease recognition sequence is specific for a restriction endonuclease that, upon digestion of the oligonucleotide library member, cuts on both sides of the Type-IIS restriction endonuclease recognition sequence. The restriction endonuclease can be a BcgI, a BsaXI or a BspCNI restriction endonuclease.

In one aspect, each oligonucleotide library member consists essentially of two codons in tandem (a dicodon) and a Type-IIS restriction endonuclease recognition sequence flanking the 5′ and the 3′ end of the dicodon.

In alternative aspects, the oligonucleotide library members are between about 20 and 400 base pairs in length, between about 40 and 200 base pairs in length or between about 100 and 150 base pairs in length.

The oligonucleotide library member can comprise a (complementary base paired) sequence (NNN)(NNN) AGAAGAGC (SEQ ID NO:1) and (NNN)(NNN) TCTTCTCG (SEQ ID NO:2), wherein (NNN) is a codon and N is A, C, T or G or an equivalent thereof.

The oligonucleotide library member can comprise a (complementary base paired) sequence (NNN)(NNN) TGAAGAGAG (SEQ ID NO:3) and (NNN)(NNN) ACTTCTCTC (SEQ ID NO:4), wherein (NNN) is a codon and N is A, C, T or G or an equivalent thereof.

The oligonucleotide library member can comprise a (complementary base paired) sequence (NNN)(NNN) TGAAGAGAG CT GCTACTAACT GCA (SEQ ID NO:5) and (NNN)(NNN) ACTTCTCTC GA CGATGATTG (SEQ ID NO:6), wherein (NNN) is a codon and N is A, C, T or G or an equivalent thereof.

The oligonucleotide library member can comprise a (complementary base paired) sequence CTCTCTTCA NNN NNN AGAAGAGC (SEQ ID NO:7) and GAGAGAAGT NNN NNN TCTTCTCG (SEQ ID NO:8), wherein (NNN) is a codon and N is A, C, T or G or an equivalent thereof.

The oligonucleotide library member can comprise a (complementary base paired) sequence CTCTCTTCA NNN NNN AGAAGAGC GGGTCTTCCAACT AGAGAATTCGATATCTGCA (SEQ ID NO:9) and GAGAGAAGT NNN NNN TCTTCTCG CCCAGAAGGTTGATCTCTTAAGCTATAG (SEQ ID NO:10), wherein (NNN) is a codon and N is A, C, T or G or an equivalent thereof.

The invention provides a method for building a polynucleotide comprising codons by iterative assembly of multicodon (e.g., dicodon) building blocks. In one aspect, the method comprises the following steps: (a) providing a library of double-stranded codon building block oligonucleotides of the invention; (b) providing a substrate surface; (c) immobilizing a first oligonucleotide member from the library of step (a) to the substrate surface of step (b) and digesting with a Type-IIS restriction endonuclease to generate a single-stranded overhang in a codon, or, digesting a first oligonucleotide member from the library of step (a) with a Type-IIS restriction endonuclease to generate a single-stranded overhang in a codon and immobilizing to the substrate surface of step (b) by the oligonucleotide end opposite the codon; (d) digesting a second oligonucleotide member from the library of step (a) with a Type-IIS restriction endonuclease to generate a single-stranded overhang in a codon; and (e) contacting the digested second oligonucleotide member of step (d) to the digested immobilized first oligonucleotide member of step (c) under conditions wherein complementary single-stranded base overhangs of the first and the second oligonucleotides can pair, and, ligating the second oligonucleotide to the first oligonucleotide; thereby building a polynucleotide comprising codons by iterative assembly of multicodon (e.g., dicodon) building blocks.

The methods of the invention can further comprise digesting the immobilized oligonucleotide of step (e) with a Type-IIS restriction endonuclease to generate a single-stranded overhang in a codon, wherein the Type-IIS restriction endonuclease recognizes a restriction endonuclease recognition sequence in the oligonucleotide distal to the substrate surface. The methods of the invention can further comprise digesting another oligonucleotide member from the library of step (a) with a Type-IIS restriction endonuclease to generate a single-stranded overhang in a codon. The methods of the invention can further comprise contacting a digested oligonucleotide library member to a digested immobilized oligonucleotide member under conditions wherein complementary single-stranded base overhangs of the oligonucleotides can pair, and, ligating the oligonucleotides; thereby building a polynucleotide comprising codons by iterative assembly of multicodon (e.g., dicodon) building blocks.

In one aspect, the method is repeated iteratively, thereby building a polynucleotide comprising a plurality of codons. The method can be iteratively repeated n times, wherein n is an integar between 2 and 10⁶ or more. The method can iteratively repeated n times, wherein n is an integer between 10² and 10⁵.

In one aspect, a member of the library is randomly selected for iterative assembly to the polynucleotide. All or a subset of the members of the library added to the polynucleotide can be selected randomly.

In one aspect, a member of the library is non-stochastically selected for iterative assembly to the polynucleotide. All or a subset of the members of the library added to the polynucleotide can be selected non-stochastically.

In one aspect, the library of oligonucleotides comprises all possible codon combinations, e.g., dimer (dicodon) combinations, tricodon combinations and the like. In one aspect, the library of oligonucleotides consists of 4096 codon dimer (dicodon) combinations. In one aspect, the codons are not stop codons.

In one aspect, the substrate surface comprises a solid surface. The solid surface can comprise a bead. The solid surface can comprise a polystyrene or a glass. In one aspect, the solid surface comprises a double-orificed container. The double-orificed container can comprise a double-orificed capillary array. The double-orificed capillary array can be a GIGAMATRIX™ capillary array.

In one aspect, the substrate surface of step (b) further comprises an immobilized double-stranded oligonucleotide. The immobilized double-stranded oligonucleotide can further comprise a codon building block oligonucleotide library member of the invention. The codon building block oligonucleotide library member can be immobilized to the immobilized double-stranded oligonucleotide by blunt end ligation.

In one aspect, the immobilized double-stranded oligonucleotide comprises a single-stranded base overhang at the non-immobilized end of the oligonucleotide. The oligonucleotide library member can be immobilized to the immobilized double-stranded oligonucleotide by base pairing of single stranded base overhangs followed by ligation.

In one aspect, the Type-IIS restriction endonuclease recognition sequence at the 5′ end of the multicodon (e.g., dicodon) differs from the Type-IIS restriction endonuclease recognition sequence at the 3′ end of the multicodon (e.g., dicodon).

In one aspect, the Type-IIS restriction endonuclease upon digestion of the oligonucleotide library member generates a three base single-stranded overhang. The Type-IIS restriction endonuclease comprises a SapI restriction endonuclease or an isochizomer thereof, or, an EarI restriction endonuclease or an isochizomer thereof.

In one aspect, the Type-IIS restriction endonuclease upon digestion of the oligonucleotide library member generates a two base single-stranded overhang. The Type-IIS restriction endonuclease can be a BseRI, a BsgI or a BpmI restriction endonuclease or an isochizomer thereof

In one aspect, the Type-IIS restriction endonuclease upon digestion of the oligonucleotide library member generates a one base single-stranded overhang. The Type-IIS restriction endonuclease can be a N.AlwI or a N.BstNBI restriction endonuclease or an isochizomer thereof.

In one aspect, the Type-IIS restriction endonuclease upon digestion of the oligonucleotide library member cuts on both sides of the Type-IIS restriction endonuclease recognition sequence. The Type-IIS restriction endonuclease can be a BcgI, a BsaXI or a BspCNI restriction endonuclease or an isochizomer thereof.

In one aspect, each library member consists essentially of two codons in tandem (a dicodon) and a Type-IIS restriction endonuclease recognition sequence flanking the 5′ and the 3′ end of the dicodon. In alternative aspects, each library member can be three, four, five, six or more codons in tandem and a Type-IIS restriction endonuclease recognition sequence flanking the 5′ and the 3′ end of the multicodon.

In alternative aspects, the oligonucleotide library members are between about 20 and 400 or more base pairs in length, between about 40 and 200 base pairs in length, between about 100 and 150 base pairs in length.

In one aspect, an oligonucleotide library member comprises a sequence (NNN)(NNN) AGAAGAGC (SEQ ID NO:1) and (NNN)(NNN) TCTTCTCG (SEQ ID NO:2), wherein (NNN) is a codon and N is A, C, T or G or an equivalent thereof.

In one aspect, an oligonucleotide library member comprises a sequence (NNN)(NNN) TGAAGAGAG (SEQ ID NO:3) and (NNN)(NNN) ACTTCTCTC (SEQ ID NO:4), wherein (NNN) is a codon and N is A, C, T or G or an equivalent thereof.

In one aspect, an oligonucleotide library member comprises a sequence (NNN)(NNN) TGAAGAGAG CT GCTACTAACT GCA (SEQ ID NO:5) and (NNN)(NNN) ACTTCTCTC GA CGATGATTG (SEQ ID NO:6), wherein (NNN) is a codon and N is A, C, T or G or an equivalent thereof.

In one aspect, an oligonucleotide library member comprises a sequence CTCTCTTCA NNN NNN AGAAGAGC (SEQ ID NO:7) and GAGAGAAGT NNN NNN TCTTCTCG (SEQ ID NO:8), wherein (NNN) is a codon and N is A, C, T or G or an equivalent thereof.

In one aspect, an oligonucleotide library member comprises a sequence CTCTCTTCA NNN NNN AGAAGAGC GGGTCTTCCAACTAGAGAATTCGAT ATCTGCA (SEQ ID NO:9) and GAGAGAAGT NNN NNN TCTTCTCG CCCAGA AGGTTGATCTCTTAAGCTATAG (SEQ ID NO:10), wherein (NNN) is a codon and N is A, C, T or G or an equivalent thereof.

In one aspect, the immobilized double-stranded oligonucleotide comprises a general formula: [Substrate] (linker) (promoter) (restriction site)(single stranded overhang). In one aspect, the immobilized double-stranded oligonucleotide comprises a general formula: (Y)n (promoter) (restriction site)(single stranded overhang), wherein Y is any nucleotide base and n is an integer between 2 and 50, or more. Any promoter can be used, e.g., constitutive or inducible. In one aspect, the promoter is a T6 promoter, a T3 promoter or an SP6 promoter. In one aspect, the promoter is directly attached to a substrate, or, is attached by a linker, which can be (Y)n nucleotide bases. The attachment to the substrate (the immobilization) can be direct or indirect, e.g., by covalent attachment or by hybridization of complementary base pairs.

In one aspect, an immobilized double-stranded oligonucleotide comprises a sequence (NNN) (NNN) CGCGCG(Y)nCGAATTGGAGCTC (SEQ ID NO:11) and (NNN) (NNN) GCGCGC(Y)nGCTTAACCTCGAGCCCC (SEQ ID NO:12), wherein n is an integer greater than or equal to 1, Y is any nucleoside and (NNN) is a codon.

In one aspect, an immobilized double-stranded oligonucleotide comprises a sequence (NNN) (NNN) CGCGCGTAATACGACTCACTATAGGGCGAATTG GAGCTC (SEQ ID NO:13) and (NNN) (NNN) and GCGCGCATTATGCTGAGTGA TATCCCGCTTAACCTCGACCCC SEQ ID NO:14).

In one aspect, an immobilized double-stranded oligonucleotide comprises a promoter. The promoter can comprise a bacteriophage promoter, such as a T7 promoter, a T6 promoter or an SP6 promoter.

In one aspect, ligating the oligonucleotides comprises use of an enzyme, such as a ligase. Any ligase can be used, such as a mammalian or a bacteria DNA ligase, including, e.g., a T4 ligase or an E. coli ligase.

In one aspect, the methods of the invention further comprise sequencing the constructed polynucleotide. The methods of the invention can further comprise determining whether all or part of the polynucleotide sequence encodes a peptide or a polypeptide. The methods of the invention can further comprise isolating the constructed polynucleotide. The methods of the invention can further comprise polymerase-based amplification of the constructed polynucleotide. The polymerase-based amplification can be a polymerase chain reaction (PCR). The methods of the invention can further comprise transcription of the constructed polynucleotide.

In one aspect, the solid substrate comprises a double-orificed container. The double-orificed container can comprise a double-orificed capillary array. The double-orificed capillary array can be a GIGAMATRIX™ capillary array.

The invention provides a multiplexed system for building a polynucleotide comprising codons by iterative assembly of codon building blocks comprising the following components: (a) a library comprising oligonucleotide members, wherein each oligonucleotide member comprises multiple codons in tandem, e.g., two codons in tandem (a dicodon), and a Type-IIS restriction endonuclease recognition sequence flanking the 5′ and the 3′ end of the multicodon (e.g., dicodon); and, (b) a substrate surface comprising a plurality of oligonucleotide library members of step (a) immobilized to the substrate surface.

The invention provides multiplexed systems for building polynucleotide comprising codons by iterative assembly of oligonucleotides comprising the following components: (a) a library of oligonucleotides of the invention; and (b) a substrate surface comprising a plurality of oligonucleotides of step (a) immobilized to the substrate surface. In one aspect, the substrate surface can further comprise a double-orificed capillary array. The double-orificed capillary array can comprise a GIGAMATRIX™ capillary array. The multiplexed system can further comprise instructions comprising all or part of a method of the invention. The substrate surface can comprise a plurality of beads, such as magnetic beads. In one aspect, the plurality of beads comprises 61 sets of beads, each comprising an oligonucleotide comprising a dicodon, one bead set for each possible amino acid coding triplet.

The invention provides kits comprising a plurality of beads sets, each bead set comprising an immobilized oligonucleotide comprising a multicodon, wherein each multicodon is flanked by a Type-IIS restriction endonuclease recognition sequence on its non-immobilized end.

The invention provides kits comprising a plurality of beads comprising 61 sets of beads, each bead comprising an immobilized oligonucleotide comprising an amino acid coding triplet, one bead set for each possible amino acid coding triplet, wherein each possible amino acid coding triplet is flanked by a Type-IIS restriction endonuclease recognition sequence on its non-immobilized end. In one aspect, an immobilized oligonucleotide comprises a promoter. The promoter can comprise a bacteriophage promoter, such as a T7 promoter, a T6 promoter or an SP6 promoter. In one aspect, the kits further comprise an enzyme, such as a ligase, e.g., a mammalian or a bacteria DNA ligase, including, e.g., a T4 ligase or an E. coli ligase.

These nucleic acids can be further manipulated or altered by any means, including random or stochastic methods, or, non-stochastic, or “directed evolution.” For example, these nucleic acids can be manipulated by saturation mutagenesis, an optimized directed evolution system, synthetic ligation reassembly, or a combination thereof, as described herein. These nucleic acids can be manipulated by a method comprising gene site saturated mutagenesis (GSSM), step-wise nucleic acid reassembly, error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, synthetic ligation reassembly (SLR) or a combination thereof. These nucleic acids can be manipulated by recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation or a combination thereof.

Chimeric Antigen Binding Molecules and Methods for Making and Using them

The invention provides a library of chimeric nucleic acids encoding a plurality of chimeric antigen binding polypeptides, the library made by a method comprising the following steps: (a) providing a plurality of nucleic acids encoding a lambda light chain variable region polypeptide domain (V_{λ}) or a kappa light chain variable region polypeptide domain (V_{κ}); (b) providing a plurality of oligonucleotides encoding a J region polypeptide domain (V_{J}); (c) providing a plurality of nucleic acids encoding a lambda light chain constant region polypeptide domain (C_{λ}) or a kappa light chain constant region polypeptide domain (C_{κ}); (d) joining together a nucleic acid of step (a), a nucleic acid of step (c) and an oligonucleotide of step (b), wherein the oligonucleotide of step (b) is placed between the nucleic acids of step (a) and step (c) to generate a V-J-C chimeric nucleic acid coding sequence encoding a chimeric antigen binding polypeptide, and repeating this joining step to generate a library of chimeric nucleic acid coding sequences encoding a library of chimeric antigen binding polypeptides.

In alternative aspects of the invention, an antigen binding polypeptide comprises a single chain antibody, a Fab fragment, an Fd fragment or an antigen binding complementarity determining region (CDR).

The lambda light chain variable region polypeptide domain (Vλ) nucleic acid coding sequence or the kappa light chain variable region polypeptide domain (Vκ) nucleic acid coding sequence of step (a) can be generated by an amplification reaction. The lambda light chain constant region polypeptide domain (Cλ) nucleic acid coding sequence or the kappa light chain constant region polypeptide domain (Cκ) nucleic acid coding sequence of step (c) also can be generated by an amplification reaction. Any amplification reaction or system can be used. The amplification reaction can comprise a polymerase chain reaction (PCR) amplification reaction using a pair of oligonucleotide primers. The amplification reaction can comprise a ligase chain reaction (LCR), a transcription amplification, a self-sustained sequence replication, a Q Beta replicase amplification and other RNA polymerase mediated techniques. In one aspects, the oligonucleotide primers can further comprise one or more restriction enzyme sites.

In alternative aspects, the lambda light chain variable region polypeptide domain (Vλ) nucleic acid coding sequence, the kappa light chain variable region polypeptide domain (Vκ) nucleic acid coding sequence, the lambda light chain constant region polypeptide domain (Cλ) nucleic acid coding sequence or the kappa light chain constant region polypeptide domain (Cκ) nucleic acid coding sequence are between about 99 and about 600 base pair residues in length, between about 198 and about 402 base pair residues in length and between about 300 and about 320 base pair residues in length.

In one aspect, the amplified nucleic acid is a mammalian nucleic acid, such as a human or a mouse nucleic acid. The amplified nucleic acid can be a genomic DNA, a cDNA or an RNA.

In alternative aspects, an oligonucleotide encoding a J region polypeptide domain of step (b) is between about 9 and about 99 base pair residues in length, between about 18 and about 81 base pair residues in length and between about 36 and about 63 base pair residues in length.

In alternative aspects, the joining step to generate a chimeric nucleic acid comprises a DNA ligase, a transcription or an amplification reaction. The amplification reaction can comprise a polymerase chain reaction (PCR) amplification reaction, a ligase chain reaction (LCR), a transcription amplification, a self-sustained sequence replication, a Q Beta replicase amplification and other RNA polymerase mediated techniques. The amplification reaction can comprise use of oligonucleotide primers. The oligonucleotide primers can further comprise a restriction enzyme site. The transcription can comprise a DNA polymerase transcription reaction.

The invention provides a library of chimeric nucleic acids encoding a plurality of chimeric antigen binding polypeptides, the library made by a method comprising the following steps: (a) providing a plurality of nucleic acids encoding an antibody heavy chain variable region polypeptide domain (V_{H}); (b) providing a plurality of oligonucleotides encoding a D region polypeptide domain (V_{D}); (c) providing a plurality of oligonucleotides encoding a J region polypeptide domain (V_{J}); (d) providing a plurality of nucleic acids encoding a heavy chain constant region polypeptide domain (C_{H}); (e) joining together a nucleic acid of step (a), a nucleic acid step (d) and an oligonucleotide of step (b) and step (c), wherein the oligonucleotides of step (b) and step (c) are placed between the nucleic acids of step (a) and step (d) to generate a V-D-J-C chimeric nucleic acid coding sequence encoding a chimeric antigen binding polypeptide, and repeating this joining step to generate a library of chimeric nucleic acid coding sequences encoding a library of chimeric antigen binding polypeptides.

In alternative aspects, the antigen binding polypeptide comprises an single chain antibody, a Fab fragment, an Fd fragment or an antigen binding complementarity determining region (CDR). The antigen binding polypeptide can comprise a μ, γ, γ2, γ3, γ4, δ, ε, α1 or α2 constant region. The heavy chain variable region polypeptide domain (V_{H}) or the heavy chain constant region polypeptide domain (CH) nucleic acid coding sequence can be generated by an amplification reaction. The amplification reaction can comprise a polymerase chain reaction (PCR) amplification reaction, a ligase chain reaction (LCR), a transcription amplification, a self-sustained sequence replication, a Q Beta replicase amplification and other RNA polymerase mediated techniques. The amplification reaction can comprise using a pair of oligonucleotide primers. The oligonucleotide primers can further comprise a restriction enzyme site.

In alternative aspects, the heavy chain variable region polypeptide domain (V_{H}) nucleic acid coding sequence or the heavy chain constant region polypeptide domain (C_{H}) nucleic acid coding sequence is between about 99 and about 600 base pair residues in length, between about 198 and about 402 base pair residues in length, or between about 300 and about 320 base pair residues in length.

The amplified nucleic acid can be a mammalian nucleic acid, such as a human or a mouse nucleic acid. The amplified nucleic acid can be a genomic DNA, a cDNA or an RNA, e.g., an mRNA.

In alternative aspects, the oligonucleotide encoding a D region polypeptide domain of step (b) or a J region polypeptide domain of step (c) is between about 9 and about 99 base pair residues in length, between about 18 and about 81 base pair residues in length, or between about 36 and about 63 base pair residues in length.

The joining of step (e) to generate a chimeric nucleic acid can comprise a DNA ligase, a transcription or an amplification reaction. The amplification reaction comprises a polymerase chain reaction (PCR) amplification reaction, a ligase chain reaction (LCR), a transcription amplification, a self-sustained sequence replication, a Q Beta replicase amplification and other RNA polymerase mediated techniques. The amplification reaction can comprise use of oligonucleotide primers. The oligonucleotide primers can further comprise a restriction enzyme site. The transcription can comprise a DNA polymerase transcription reaction.

The invention provides an expression vector comprising a chimeric nucleic acid selected from a library of the invention. The invention provides a transformed cell comprising a chimeric nucleic acid selected from a library of the invention. The invention provides a transformed cell comprising an expression vector of the invention. The invention provides a non-human transgenic animal comprising a chimeric nucleic acid selected from a library of the invention.

The invention provides a method for making a chimeric antigen binding polypeptide comprising the following steps: (a) providing a nucleic acid encoding a lambda light chain variable region polypeptide domain (V_{λ}) or a kappa light chain variable region polypeptide domain (V_{κ}); (b) providing an oligonucleotides encoding a J region polypeptide domain (V_{J}); (c) providing a nucleic acid encoding a lambda light chain constant region polypeptide domain (C_{λ}) or a kappa light chain constant region polypeptide domain (C_{κ}); (d) joining together a nucleic acid of step (a), a nucleic acid of step (c) and an oligonucleotide of step (b), wherein the oligonucleotide of step (b) is placed between the nucleic acids of step (a) and step (c) to generate a V-J-C chimeric nucleic acid coding sequence encoding a chimeric antigen binding polypeptide.

The invention provides a method for making a library of chimeric antigen binding polypeptides comprising the following steps: (a) providing a plurality of nucleic acids encoding a lambda light chain variable region polypeptide domain (V_{λ}) or a kappa light chain variable region polypeptide domain (V_{κ}); (b) providing a plurality of oligonucleotides encoding a J region polypeptide domain (V_{J}); (c) providing a plurality of nucleic acids encoding a lambda light chain constant region polypeptide domain (C_{λ}) or a kappa light chain constant region polypeptide domain (C_{κ}); (d) joining together a nucleic acid of step (a), a nucleic acid of step (c) and an oligonucleotide of step (b), wherein the oligonucleotide of step (b) is placed between the nucleic acids of steps (a) and step (c) to generate a V-J-C chimeric nucleic acid coding sequence encoding a chimeric antigen binding polypeptide, and repeating this joining step to generate a library of chimeric nucleic acid coding sequences encoding a library of chimeric antigen binding polypeptides.

The invention provides a method for making a chimeric antigen binding polypeptide comprising the following steps: (a) providing a nucleic acid encoding an antibody heavy chain variable region polypeptide domain (V_{H}); (b) providing an oligonucleotide encoding a D region polypeptide domain (V_{D}); (c) providing an oligonucleotide encoding a J region polypeptide domain (V_{J}); (d) providing a nucleic acid encoding a heavy chain constant region polypeptide domain (C_{H}); (e) joining together a nucleic acid of step (a), a nucleic acid of step (d) and an oligonucleotide of step (b) and step (c), wherein the oligonucleotides of step (b) and step (c) are placed between the nucleic acids of step (a) and step (d) to generate a V-D-J-C chimeric nucleic acid coding sequence encoding a chimeric antigen binding polypeptide.

The invention provides a method for making a library of chimeric antigen binding polypeptides comprising the following steps: (a) providing a plurality of nucleic acids encoding an antibody heavy chain variable region polypeptide domain (V_{H}); (b) providing a plurality of oligonucleotides encoding a D region polypeptide domain (V_{D}); (c) providing a plurality of oligonucleotides encoding a J region polypeptide domain (V_{J}); (d) providing a plurality of nucleic acids encoding a heavy chain constant region polypeptide domain (C_{H}); (e) joining together a nucleic acid of step (a), a nucleic acid of step (d) and an oligonucleotide of step (b) and step (c), wherein the oligonucleotides of step (b) and step (c) are placed between the nucleic acids of step (a) and step (d) to generate a V-D-J-C chimeric nucleic acid coding sequence encoding a chimeric antigen binding polypeptide, and repeating this joining step to generate a library of chimeric nucleic acid coding sequences encoding a library of chimeric antigen binding polypeptides.

The methods the invention can further comprise expressing the nucleic acid coding sequences encoding one or a library of chimeric antigen binding polypeptides. The methods the invention can further comprise screening the expressed chimeric antigen binding polypeptide for its ability to specifically bind an antigen.

The methods the invention can further comprise mutagenizing the nucleic acid coding sequence encoding a chimeric antigen binding polypeptide by a method comprising an optimized directed evolution system or a synthetic ligation reassembly, saturation mutagenesis, or a combination thereof. The methods the invention can further comprise screening the mutagenized chimeric antigen binding polypeptide for its ability to specifically bind an antigen. The methods the invention can further comprise screening the mutagenized chimeric antigen binding polypeptide for its ability to specifically bind an antigen. The methods the invention can further comprise identifying a mutagenized antigen binding site variant by its increased antigen binding affinity or antigen binding specificity as compared to the affinity or specificity of the chimeric antigen binding polypeptide before mutagenesis. The methods the invention can further comprise screening the mutagenized chimeric antigen binding polypeptide for its ability to specifically bind an antigen by a method comprising phage display of the antigen binding site polypeptide. The methods the invention can further comprise screening the mutagenized chimeric antigen binding polypeptide for its ability to specifically bind an antigen by a method comprising expression of the expressed antigen binding site polypeptide in a liquid phase. The methods the invention can further comprise screening the mutagenized chimeric antigen binding polypeptide for its ability to specifically bind an antigen by a method comprising ribosome display of the antigen binding site polypeptide. The methods the invention can further comprise screening the chimeric antigen binding polypeptide for its ability to specifically bind an antigen by a method comprising immobilizing the polypeptide in a solid phase. The methods the invention can further comprise screening the chimeric antigen binding polypeptide for its ability to specifically bind an antigen by a method comprising a capillary array. The methods the invention can further comprise screening the chimeric antigen binding polypeptide for its ability to specifically bind an antigen by a method comprising a double-orificed container. The double-orificed container can comprise a double-orificed capillary array. The double-orificed capillary array can be a GIGAMATRIX™ capillary array.

The method provides a method for making a library of chimeric antigen binding polypeptides comprising the following steps: (a) providing a plurality of V-J-C chimeric nucleic acids encoding a chimeric antigen binding polypeptide made by a method as set forth in claim 48 or a plurality of V-D-J-C chimeric nucleic acids encoding a chimeric antigen binding polypeptide made by a method as set forth in claim 50; (b) providing a plurality of oligonucleotides, wherein each oligonucleotide comprises a sequence homologous to a chimeric nucleic acid of step (a), thereby targeting a specific sequence of the chimeric nucleic acid, and a sequence that is a variant of the chimeric nucleic acid; and (c) generating “n” number of progeny polynucleotides comprising non-stochastic sequence variations by replicating the chimeric nucleic acid of step (a) with the oligonucleotides of step (b), wherein n is an integer, thereby generating a library of chimeric antigen binding polypeptides.

In alternative aspects, the sequence homologous to the chimeric nucleic acid is x bases long, wherein x is an integer between 3 and 100, between 5 and 50 and between 10 and 30. In one aspect, the sequence that is a variant of the chimeric nucleic acid is x bases long, wherein x can be an integer between 1 and 50 or between 2 and 20. The oligonucleotide of step (b) can further comprise a second sequence homologous to the chimeric nucleic acid, wherein the variant sequence is flanked by the sequences homologous to the chimeric nucleic acid. In one aspect, the second sequence that is a variant of the chimeric nucleic acid is x bases long, wherein x is an integer between 1 and 50, or, where x is 3, 6, 9 or 12.

In one aspect, the oligonucleotides can comprise variant sequences targeting a chimeric nucleic acid codon, thereby generating a plurality of progeny chimeric polynucleotides comprising a plurality of variant codons. The variant sequences can generate variant codons encoding all nineteen naturally-occurring amino acid variants for a targeted codon, thereby generating all nineteen possible natural amino acid variations at the residue encoded by the targeted codon. The oligonucleotides can comprise variant sequences targeting a plurality of chimeric nucleic acid codons. The oligonucleotides can comprise variant sequences targeting all of the codons in the chimeric nucleic acid, thereby generating a plurality of progeny polypeptides wherein all amino acids are non-stochastic variants of the polypeptide encoded by the chimeric nucleic acid. The variant sequences can generate variant codons encoding all nineteen naturally-occurring amino acid variants for all of the chimeric nucleic acid codons, thereby generating a plurality of progeny polypeptides wherein all amino acids are non-stochastic variants of the polypeptide encoded by the chimeric nucleic acid and a variant for all nineteen possible natural amino acids at all of the codons.

In alternative aspects of the methods, in generating “n” number of progeny polynucleotides comprises non-stochastic sequence variations, “n” is an integer between 1 and about 10³⁰, between about 10² and about 10²⁰, or between about 10² and about 10¹⁰.

In alternative aspects of the methods, the replicating of step (c) comprises an enzyme-based replication, such as a polymerase-based amplification reaction. The amplification reaction can comprise a polymerase chain reaction (PCR). The enzyme-based replication can comprise an error-free polymerase reaction.

In one aspect of the methods, an oligonucleotide of step (b) further comprises a nucleic acid sequence capable of introducing one or more nucleotide residues into the template polynucleotide. The oligonucleotide of step (b) can further comprise a nucleic acid sequence capable of deleting one or more residue from the template polynucleotide. The oligonucleotide of step (b) can further comprise addition of one or more stop codons to the template polynucleotide.

The invention provides a method for making a library of chimeric antigen binding polypeptides comprising the following steps: (a) providing x number of V-J-C chimeric nucleic acids encoding a chimeric antigen binding polypeptide made by a method as set forth in claim 48 or x number of V-D-J-C chimeric nucleic acids encoding a chimeric antigen binding polypeptide made by a method as set forth in claim 50; (b) providing y number of building block polynucleotides, wherein y is an integer, and the building block polynucleotides are designed to cross-over reassemble with a chimeric nucleic acid of step (a) at predetermined sequences and comprise a sequence that is a variant of the chimeric nucleic acid and a sequence homologous to the chimeric nucleic acid flanking the variant sequence; and, (c) combining at least one building block polynucleotide with at least one chimeric nucleic acid such that the building block polynucleotide cross-over reassembles with the chimeric nucleic acid to generate non-stochastic progeny chimeric polynucleotides, thereby generating a library of polynucleotides encoding chimeric antigen binding polypeptides.

In alternative aspects of the method, x is an integer between 1 and about 10¹⁰, or between about 10 and about 10², or, x is an integer selected from the group consisting of 1, 2, 3, 4, 5, 6, 7, 8, 9 and 10.

In one aspect, a plurality of building block polynucleotides are used and the variant sequences target a chimeric nucleic acid codon to generate a plurality of progeny polynucleotides that are variants of the targeted codon, thereby generating a plurality of natural amino acid variations at a residue in a polypeptide encoded by the chimeric nucleic acid. In one aspect, the variant sequences generate variant codons encoding all nineteen naturally-occurring amino acid variants for the targeted codon, thereby generating all nineteen possible natural amino acid variations at the residue encoded by the targeted codon in a polypeptide encoded by the chimeric nucleic acid.

In one aspect, a plurality of building block polynucleotides are used, and the variant sequences target a plurality of chimeric nucleic acid codons, thereby generating a plurality of codons that are variants of the targeted codons and a plurality of natural amino acid variations at a plurality of residues encoded by the targeted codon in a polypeptide encoded by the chimeric nucleic acid. In one aspect, the variant sequences generate variant codons in all of the codons in the chimeric nucleic acid, thereby generating a plurality of progeny polypeptides wherein all amino acids are non-stochastic variants of the polypeptide encoded by the chimeric nucleic acid. In one aspect, the variant sequences generate variant codons encoding all nineteen naturally-occurring amino acid variants for all of the chimeric nucleic acid codons, thereby generating a plurality of progeny polypeptides wherein all amino acids are non-stochastic variants of the polypeptide encoded by the chimeric nucleic acid and a variant for all nineteen possible natural amino acids at all of the codons. In one aspect, all of the codons in an antigen binding site are targeted.

In alternative aspects, the library comprises between 1 and about 10³⁰ members, between about 10² and about 10²⁰ members or between about 10³ and about 10¹⁰ members. In alternative aspects, an end of a building block polynucleotide comprises at least about 6 nucleotides homologous to a chimeric nucleic acid, at least about 15 nucleotides homologous to a chimeric nucleic acid or at least about 21 nucleotides homologous to a chimeric nucleic acid.

In one aspect, combining one or more building block polynucleotides with a chimeric nucleic acid comprises z cross-over events between the building block polynucleotides and the chimeric nucleic acid, wherein y is an integer between 1 and about 10²⁰, between about 10 and about 10¹⁰, or between about 10² and about 10⁵.

In alternative aspects, a non-stochastic progeny chimeric polynucleotide differs from a chimeric nucleic acid in z number of residues, wherein z is between 1 and about 10⁴ or between 10 and about 10³, or, z is 1, 2, 3, 4, 5, 6, 7, 8, 9 and 10.

In alternative aspects, a non-stochastic progeny chimeric polynucleotide differs from a chimeric nucleic acid in z number of codons, wherein z is between 1 and about 10⁴, z is between 10 and about 10³., or z is 1, 2, 3, 4, 5, 6, 7, 8, 9 and 10.

In alternative aspects, the methods of the invention further comprise non-stochastic modification of all or a part of the sequence of a chimeric antibody coding sequence of the invention. The modification can be by any method, including, e.g., by “saturation mutagenesis” or “GSSM, ” “optimized directed evolution system” and “synthetic ligation reassembly” or “SLR” or any combination of these methods.

Nucleic acids encoding the chimeric antibodies of the invention can be further manipulated or altered by any means, including random or stochastic methods, or, non-stochastic, or “directed evolution.” For example, nucleic acids encoding the chimeric antibodies of the invention can be manipulated by step-wise nucleic acid reassembly (see Example 3, below), saturation mutagenesis, an optimized directed evolution system, synthetic ligation reassembly, or a combination thereof, as described herein. Nucleic acids encoding the chimeric antibodies of the invention can be manipulated by a method comprising gene site saturated mutagenesis (GSSM), error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, synthetic ligation reassembly (SLR) or a combination thereof. These nucleic acids can be manipulated by recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation or a combination thereof.

The details of one or more embodiments of the invention are set forth in the accompanying drawings and the description below. Other features, objects, and advantages of the invention will be apparent from the description and drawings, and from the claims.

All publications, GenBank Accession references (sequences), ATCC Deposits, patents and patent applications cited herein are hereby expressly incorporated by reference for all purposes.

### DESCRIPTION OF DRAWINGS

FIG. 1 schematically illustrates an exemplary “elongation cycle” of a gene building method of the invention, the method comprising: “loading” starter oligo onto substrate; ligation (with any ligase, e.g., T4 ligase or E. coli ligase); wash; fill-in ends; wash; cut with restriction endonuclease; wash; repeat (reiterate cycle), as discussed in detail in the Example 1, below.

FIG. 2 schematically illustrates a cloning vector designed to reassemble antibody light chains according the methods of the invention, as discussed in Example 2.

FIG. 3 schematically illustrates an exemplary scheme to reassemble lambda light chains according the methods of the invention, as discussed in Example 2.

FIG. 4 schematically illustrates an exemplary scheme to reassemble kappa light chains according the methods of the invention, as discussed in Example 2.

FIG. 5 schematically illustrates an exemplary scheme to reassemble antibody heavy chains according the methods of the invention, as discussed in Example 2.

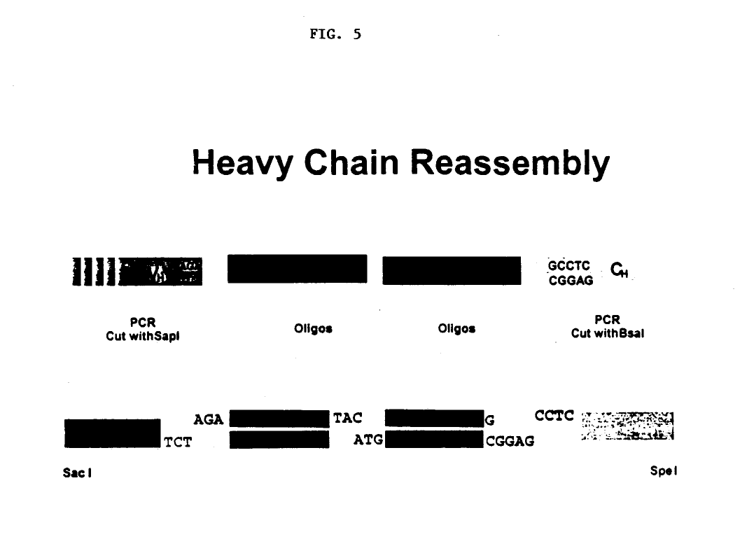

FIG. 6 illustrates an exemplary procedure for the reassembly of three esterase genes, as discussed in Example 3.

FIG. 7A illustrates the elution of reassembled DNA from the solid support using alternative restriction sites engineered in the biotinylated hook, as discussed in Example 3. FIG. 7B illustrates the elution of final reassembled products from the solid support, as discussed in Example 3.

FIG. 8 illustrates an exemplary software program used in the methods of the invention.

Like reference symbols in the various drawings indicate like elements.

### DETAILED DESCRIPTION

Methods for Purifying and Identifying Double-stranded Nucleic Acids Lacking Base Pair Mismatches, Insertion/deletion Loops or Nucleotide Gaps

The invention provides methods for identifying and purifying double-stranded polynucleotides lacking nucleotide gaps, base pair mismatches and insertion/deletion loops.

Definitions

Unless defined otherwise, all technical and scientific terms used herein have the meaning commonly understood by a person skilled in the art to which this invention belongs. As used herein, the following terms have the meanings ascribed to them unless specified otherwise.

The phrase “polypeptides that specifically bind to a nucleotide gap or gaps, a base pair mismatch and/or an insertion/deletion loop in a double stranded polynucleotide”include all polypeptides, natural or synthetic, that can specifically bind to a nucleoside base pair mismatch, an insertion/deletion loop and/or a nucleotide gap or gaps in a double stranded polynucleotide (e.g., oligonucleotide). These polypeptides include, e.g., DNA repair enzymes, antibodies, transcriptional regulatory polypeptides and the like, as described in further detail herein. Specifically binds means any level of affinity of binding that is not non-specific.

The phrase “lacking base pair mismatches, insertion/deletion loops and/or a nucleotide gap or gaps” means substantially lacking or completely lacking base pair mismatches, insertion/deletion loops and/or a nucleotide gap or gaps. For example, the methods of the invention can generate a sample or “batch” of purified oligonucleotides and/or polynucleotides that are 90%, 95%, 96%, 97%, 98%, 99%, 99.5%, 99.9% and 100% or completely free of base pair mismatches, insertion/deletion loops and/or nucleotide gaps.

The phrase “DNA repair enzymes” includes all DNA repair enzymes and natural or synthetic (e.g., genetically reengineered) variations thereof that can specifically bind to a base pair mismatch, an insertion/deletion loop and/or a nucleotide gap or gaps in a double stranded polynucleotide (e.g., oligonucleotide), including, e.g., DNA mismatch repair (MMR) enzymes, Taq MutS enzymes, Fpg enzymes, MutY DNA repair enzymes, hexA DNA mismatch repair enzymes, Vsr mismatch repair enzymes and the like, as described in further detail, below.

The term “MutS DNA repair enzyme” includes all MutS DNA repair enzymes, including synthetic (e.g., genitically reengineered) variations, and eukaryotic (e.g., mammalian) homologues of bacterial enzymes, that can bind a nucleoside base pair mismatch or an insertion/deletion loop, including, e.g., the Thermus aquaticus (Taq) and Pseudomonas aeruginosa MutS DNA repair enzymes, as described in further detail, below.

The term “Fpg DNA repair enzyme” includes all Fpg DNA repair enzymes, including synthetic (e.g., genetically reengineered) variations, and eukaryotic (e.g., mammalian) homologues of bacterial enzymes, that can bind a nucleoside base pair mismatch or an insertion/deletion loop, as described in further detail, below.

The term “MutY” includes all MutY DNA repair enzymes, including synthetic (e.g., genetically reengineered) variations, and eukaryotic (e.g., mammalian) homologues of bacterial enzymes, that can bind a nucleoside base pair mismatch or an insertion/deletion loop, as described in further detail, below

The term “DNA glycosylase” includes all natural or synthetic DNA glycosylase enzymes that initiate base-excision repair of G:U/T mismatches. The natural DNA glycosylase enzymes include, e.g., bacterial mismatch-specific uracil-DNA glycosylase (MUG) DNA repair enzymes and eukaryotic thymine-DNA glycosylase (TDG) enzymes, as described in further detail, below.

The term “intein” includes all polypeptide sequences that are self-splicing. Inteins are intron-like elements that are removed post-translationally by self-splicing, as described in further detail, below.

The term “saturation mutagenesis” or “GSSM” includes a method that uses degenerate oligonucleotide primers to introduce point mutations into a polynucleotide, as described in detail herein.

The term “optimized directed evolution system” or “optimized directed evolution” includes a method for reassembling fragments of related nucleic acid sequences, e.g., related genes, and explained in detail herein.

The term “synthetic ligation reassembly” or “SLR” includes a method of ligating oligonucleotide fragments in a non-stochastic fashion, and explained in detail herein.

The terms “nucleic acid” and “polynucleotide” as used herein refer to a deoxyribonucleotide or ribonucleotide in either single- or double-stranded form. The terms encompass all nucleic acids, e.g., oligonucleotides, and modifications analogues of natural nucleotides, e.g., nucleic acids with modified internucleoside linkages. The terms also encompass nucleic-acid-like structures with synthetic backbones. Synthetic backbone analogues include, e.g., phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units, and can be used as probes (see, e.g., U.S. Pat. No. 5,871,902). Phosphorothioate linkages are described, e.g., in WO 97/03211; WO 96/39154; Mata (1997) Toxicol. Appl. Pharmacol. 144:189-197. Other synthetic backbones include methyl-phosphonate linkages or alternating methylphosphonate and phosphodiester linkages (Strauss-Soukup (1997) Biochemistry 36:8692-8698), and benzylphosphonate linkages (Samstag (1996) Antisense Nucleic Acid Drug Dev 6:153-156). Modified internucleoside linkages that are resistant to nucleases are described, e.g., in U.S. Pat. No. 5,817,781. The term nucleic acid can be used interchangeably with the terms gene, cDNA, mRNA, iRNA, tRNA, primer, probe, amplification product and the like.

Base Pair Mismatch-, Insertion/deletion Loop- and Gap-binding Polypeptides

The invention provides a method for purifying double-stranded polynucleotides lacking base pair mismatches, insertion/deletion loops and/or nucleotide gaps comprising providing a plurality of polypeptides that specifically bind to a base pair mismatch, an insertion/deletion loop and/or nucleotide gaps within a double stranded polynucleotide. The methods of the invention can use any polypeptide, natural or synthetic, that specifically binds to a base pair mismatch, an insertion/deletion loop and/or a nucleotide gap or gaps in a double stranded polynucleotide. This includes all polypeptides, natural or synthetic, that can specifically bind to a nucleoside base pair mismatch, an insertion/deletion loop and/or a nucleotide gap or gaps in a double stranded polynucleotide, such as a double stranded oligonucleotide. The polypeptide can be, e.g., an enzyme, a structural protein, an antibody, variations thereof, or a protein of entirely synthetic, e.g., in silico, design. These polypeptides include, e.g., DNA repair enzymes and transcriptional regulatory polypeptides and the like. In one aspect, the mismatch or insertion/deletion loop is not within the extreme 5′ or 3′ end of the double stranded nucleic acid.

DNA repair enzymes can include all DNA repair enzymes and natural or synthetic (e.g., genetically reengineered) variations thereof that can specifically bind to a base pair mismatch, an insertion/deletion loop and/or a nucleotide gap or gaps in a double stranded polynucleotide. Examples include, e.g., DNA mismatch repair (MMR) enzymes (see, e.g., Hsieh (2001) Mutat. Res. 486(2):71-87), Taq MutS enzymes, Fpg enzymes, MutY DNA repair enzymes, hexA DNA mismatch repair enzymes (see, e.g., Ren (2001) Curr. Microbiol. 43:232-237), Vsr mismatch repair enzymes (see, e.g., Mansour (2001) Mutat. Res. 485(4):331-338) and the like. See, e.g., Mol (1999) Annu. Rev. Biophys. Biomol. Struct. 28:101-128; Obmolova (2000) Nature 407(6805):703-710.

MutS DNA repair enzymes include all MutS DNA repair enzymes, including synthetic (e.g., genetically reengineered) variations, and eukaryotic (e.g., mammalian) homologues of bacterial enzymes, that can bind a nucleoside base pair mismatch or an insertion/deletion loop, including, e.g., the Thermus aquaticus (Taq) and Pseudomonas aeruginosa MutS DNA repair enzymes. The MutS DNA repair enzyme can be used in the form of a dimer. For example, it can be a homodimer of a MutS homolog, e.g., a human MutS homolog, a murine MutS homolog, a rat MutS homolog, a Drosophila MutS homolog, a yeast MutS homolog, such as a Saccharomyces cerevisiae MutS homolog. See, e.g., U.S. Pat. No. 6,333,153; Pezza (2002) Biochem J. 361(Pt 1):87-95; Biswas (2001) J. Mol. Biol. 305:805-816; Biswas (2000) Biochem J. 347 Pt 3:881-886; Biswas (1999) J. Biol. Chem. 274:23673-23678. MutS has been shown to preferentially bind a nucleic acid heteroduplex containing a deletion of a single base, see, e.g., Biwas (1997) J. Biol. Chem. 272:13355-13364; see also, Su (1986) Proc. Natl. Acad. Sci. 83:5057-5061; Malkov (1997) J. Biol. Chem. 272:23811-23817.

Fpg DNA repair enzymes includes all Fpg DNA repair enzymes, including synthetic (e.g., genetically reengineered) variations, and eukaryotic (e.g., mammalian) homologues of bacterial enzymes, that can bind a nucleoside base pair mismatch or an insertion/deletion loop, including, e.g., the Fgp enzyme from Escherichia coli. See, e.g., Leipold (2000) Biochemistry 39:14984-14992.

MutY DNA repair enzymes include all MutY DNA repair enzymes, including synthetic (e.g., genetically reengineered) variations, and eukaryotic (e.g., mammalian) homologues of bacterial enzymes, that can bind a nucleoside base pair mismatch or an insertion/deletion loop (see, e.g., Porello (1998) Biochemistry 37:14756-14764; Williams (1999) Biochemistry 38:15417-15424).

DNA glycosylase includes all natural or synthetic DNA glycosylase enzymes that initiate base-excision repair of G:U/T mismatches. The natural DNA glycosylase enzymes form a homologous family of DNA glycosylase enzymes that initiate base-excision repair of G:U/T mismatches, including, e.g., bacterial mismatch-specific uracil-DNA glycosylase (MUG) DNA repair enzymes (see, e.g., Barrett (1999) EMBO J. 18:6599-6609) and eukaryotic thymine-DNA glycosylase (TDG) enzymes (see, e.g., Barrett (1999) ibid; Barrett (1998) Cell 92:117-129). See also Pearl (2000) Mutat. Res. 460:165-181; Niederreither (1998) Oncogene 17:1577-15785.

Additional nucleotide gap binding polypeptides include, e.g., DNA polymerase deltas, such as the DNA polymerase delta isolated in the teleost fish Misgurnus fossilis (see, e.g., Sharova (2001) Biochemistry (Mosc) 66:402-409); DNA polymerase betas, see, e.g., Bhattacharyya (2001) Biochemistry 40:9005-9013; DNA topoisomerases, such as type IB DNA topoisomerase V, as in the hyperthermophile Methanopyrus kandleri described by Belova (2001) Proc. Natl. Acad. Sci. USA 98:6015-6020; ribosomal proteins, e.g., S3 ribosomal proteins such as the Drosophila S3 ribosomal protein described by Hegde (2001) J. Biol. Chem. 276:27591-2756.

The methods of the invention comprise contacting the double-stranded polynucleotides with the polypeptides to be purified of base pair mismatches, insertion/deletion loops and/or a nucleotide gap or gaps under conditions wherein a mismatch-, an insertion/deletion loop- and/or a gap-binding polypeptide can specifically bind to a base pair mismatch or an insertion/deletion loop or a nucleotide gap or gaps. These conditions are well known in the art, as described, e.g., in the references cited herein, or, can be determined or optimized by one skilled in the art without undue experimentation. For example, U.S. Pat. No. 6,333,153, describes a method comprising contacting a MutS dimer and the mismatched duplex DNA in the presence of a binding solution comprising ADP and optionally ATP. The concentration of ATP, if present, in the binding solution is less than about 3 micromolar. The MutS dimer binds ADP, and the MutS ADP-bound dimer associates with a mismatched region of the duplex DNA.

In mammalian cells most altered bases in DNA are repaired through a single-nucleotide patch base excision repair mechanism. Base excision repair is initiated by a DNA glycosylase that removes a damaged base and generates an abasic site (AP site). This AP site is further processed by an AP endonuclease activity that incises the phosphodiester bond adjacent to the AP site and generates a strand break containing 3′-OH and 5′-sugar phosphate ends. In mammalian cells, the 5′-sugar phosphate is removed by the AP lyase activity of DNA polymerase beta. The same enzyme also fills the gap, and the DNA ends are finally rejoined by DNA ligase. Thus, in addition to DNA polymerases such as DNA polymerase beta, the methods of the invention also can use DNA glycosylases as oligonucleotide or polynucleotide binding polypeptides alone or in conjunction with other base pair mismatch-, insertion/deletion loop- or nucleotide gap-binding polypeptides. See, e.g., Podlutsky (2001) Biochemistry 40:809-813.

Marker and Selection Polypeptides

The invention provides a methods comprising purifying a double-stranded polynucleotide lacking base pair mismatches, insertion/deletion loops and/or a nucleotide gap or gaps, wherein the polynucleotide encodes a fusion protein coding sequence that comprises a coding sequence for a polypeptide of interest upstream of and in frame with a coding sequence for a marker or a selection polypeptide. The use of a marker or a selection polypeptide coding sequence downstream of and in frame with a polypeptide of interest acts to confirm that the polypeptide of interest coding sequence lacks defects that would prevent transcription or translation of the fusion protein sequence. Because the marker or a selection polypeptide coding sequence is downstream and in frame with the polypeptide of interest coding sequence, any such defects would prevent transcription and/or translation of the marker or selection polypeptide. For example, this scheme can be used to segregate or purify out polypeptide of interest coding sequences lacking base pair mismatches, insertion/deletion loops and/or a nucleotide gap or gaps from those with a defect that would prevent transcription or translation of the sequence, the defect including, e.g., base pair mismatches, insertion/deletion loops and/or gap(s).

Selection markers can be incorporated to confer a phenotype to facilitate selection of cells transformed with the sequences purified by the methods of the invention. For example, a marker selection polypeptide can comprise an enzyme, e.g., LacZ encoding a polypeptide with beta-galactosidase activity which, when expressed in a transformed cell and exposed to the appropriate substrate will produce a detectable marker, e.g., a color. See, e.g., Jain (1993) Gene 133:99-102; St Pierre (1996) Gene 169:65-68; Pessi (2001) Microbiology 147(Pt 8):1993-1995. See also U.S. Pat. Nos. 5,444,161; 4,861,718; 4,708,929; 4,668,622. Selection markers can code for episomal maintenance and replication such that integration into the host genome is not required. Selection markers can code for chloramphenicol acetyl transferase (CAT); an enzyme-substrate reaction is monitored by addition of an exogenous electron carrier and a tetrazolium salt. See, e.g., U.S. Pat. No. 6,225,074.

The marker can also encode antibiotic, herbicide or drug resistance to permit selection of those cells transformed with the desired DNA sequences. For example, antibiotic resistance can be conferred by herpes simplex thymidine kinase (conferring resistance to ganciclovir), chloramphenicol resistance enzymes (see, e.g., Harrod (1997) Nucleic Acids Res. 25:1720-1726), kanamycin resistance enzymes, aminoglycoside phosphotransferase (conferring resistance to G418), bleomycin resistance enzymes, hygromycin resistance enzymes, and the like. The marker can also encode a herbicide resistance, e.g., chlorosulfuron or Basta. Because selectable marker genes conferring resistance to substrates like neomycin or hygromycin can only be utilized in tissue culture, chemoresistance genes are also used as selectable markers in vitro and in vivo. The marker can also encode enzymes conferring resistance to a drug, e.g., an oubain-resistant (Na, K)-ATPase; a MDR1 multidrug transporter (confers resistance to certain cytotoxic drugs), and the like. Various target cells are rendered resistant to anticancer drugs by transfer of chemoresistance genes encoding P-glycoprotein, the multidrug resistance-associated protein-transporter, dihydrofolate reductase, glutathione-S-transferase, O6-alkylguanine DNA alkyltransferase, or aldehyde reductase. See, e.g., Licht (1995) Cytokines Mol. Ther. 1:11-20; Blondelet-Rouault (1997) Gene 190:315-317; Aubrecht (1997) J. Pharmacol. Exp. Ther. 281:992-997; Licht (1997) Stem Cells 15:104-111; Yang (1998) Clin. Cancer Res. 4:731-741. See also U.S. Pat. No. 5,851,804, describing chimeric kanamycin resistance genes; U.S. Pat. No. 4,784,949.

The marker or selection polypeptide can also comprise a sequence coding for a polypeptide with affinity to a known antibody to facilitate affinity purification, detection, or the like. Such detection- and purification-facilitating domains include, but are not limited to, metal chelating peptides such as polyhistidine tracts and histidine-tryptophan modules that allow purification on immobilized metals, protein A or biotin domains that allow purification, e.g., on immobilized immunoglobulin or streptavidin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between the protein of interest and the second domain can also be used, e.g., to facilitate purification and for ease of handling and using the protein of interest. For example, a fusion protein can comprise six histidine residues followed by thioredoxin and an enterokinase cleavage site (for example, see Williams (1995) Biochemistry 34:1787-1797). The histidine residues facilitate detection and purification while the enterokinase cleavage site provides a means for purifying the desired protein of interest from the remainder of the fusion protein. Technology pertaining to vectors encoding fusion proteins and application of fusion proteins are well described in the patent and scientific literature, see e.g., Kroll (1993) DNA Cell. Biol., 12:441-53.

Inteins

In one aspect, the marker or selection polypeptide coding sequence can be a self-splicing intein. Inteins are intron-like elements that are removed post-translationally by self-splicing. Thus, the methods of the invention can further comprise the self-splicing out of the marker or selection polypeptide intein coding sequence from the polypeptide of interest. Intein sequences are well known in the art. See, e.g., Colston (1994) Mol. Microbiol. 12:359-363; Perler (1994) Nucleic Acids Res. 22:1125-1127; Perler (1997) Curr. Opin. Chem. Biol. 1:292-299; Giriat (2001) Genet. Eng. (NY) 23:171-199. See also, U.S. Pat. Nos. 5,795,731; 5,496,714. For example, because inteins are protein splicing elements that occur naturally as in-frame protein fusions, intein sequences can be designed or based on naturally occurring intein sequences. Inteins are phylogenetically widespread, having been found in all three biological kingdoms, eubacteria, archaea and eukaryotes. Alternatively, they entirely synthetic splicing sequences. Intein nomenclature parallels that for RNA splicing, whereby the coding sequences of a gene (exteins) are interrupted by sequences that specify the protein splicing element (intein).

Purifying Error Free Polynucleotides

In one aspect, the methods of the invention comprise purifying double-stranded polynucleotides lacking a base pair mismatch-, an insertion/deletion loop and/or a nucleotide gap or gaps. Any purification methodology can be used, including use of antibodies, binding molecules, size exclusion and the like.

Antibodies and Immunoaffinity Columns

In one aspect, antibodies are used to purify a double-stranded polynucleotide lacking a base pair mismatch-, an insertion/deletion loop or a nucleotide gap or gaps. For example, antibodies can be designed to specifically bind directly to a base pair mismatch-, insertion/deletion loop- or nucleotide gap-binding polypeptide, or, antibodies can bind to an epitope bound to the base pair mismatch-, insertion/deletion loop- or nucleotide gap-binding polypeptide. The antibody can be bound to a bead, such as a magnetized bead. See, e.g., U.S. Pat. Nos. 5,981,297; 5,508,164; 5,445,971; 5,445,970. See also, U.S. Pat. Nos. 5,858,223; 5,746,321, and, U.S. Pat. No. 6,312,910, describing a multistage electromagnetic separator to separate magnetically susceptible materials suspended in fluids.

The separating can comprise use of an immunoaffinity column, wherein the column comprises immobilized antibodies capable of specifically binding to the specifically bound base pair mismatch-, insertion/deletion loop- or nucleotide gap-binding polypeptide or an epitope bound to the base pair mismatch-, insertion/deletion loop- or nucleotide gap-binding polypeptide. The sample is passed through an immunoaffinity column under conditions wherein the immobilized antibodies are capable of specifically binding to the specifically bound polypeptide or the epitope, or “tag,” bound to the specifically bound polypeptide.

Monoclonal or polyclonal antibodies to base pair mismatch-, insertion/deletion loop-binding and/or a nucleotide gap-binding polypeptides can be used. Methods of producing polyclonal and monoclonal antibodies are known to those of skill in the art and described in the scientific and patent literature, see, e.g., Coligan, Current Protocols in Immunology, Wiley/Greene, NY (1991); Stites (eds.) Basic and Clinical Immunology (7th ed.) Lange Medical Publications, Los Altos, Calif. (“Stites”); Goding, Monoclonal Antibodies: Principles and Practice (2d ed.) Academic Press, New York, N.Y. (1986); Kohler (1975) Nature 256:495; Harlow (1988) Antibodies, a Laboratory Manual, Cold Spring Harbor Publications, New York. Antibodies also can be generated in vitro, e.g., using recombinant antibody binding site expressing phage display libraries, in addition to the traditional in vivo methods using animals. See, e.g., Huse (1989) Science 246:1275; Ward (1989) Nature 341:544; Hoogenboom (1997) Trends Biotechnol. 15:62-70; Katz (1997) Annu. Rev. Biophys. Biomol. Struct. 26:27-45.

The term “antibody” includes a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

Biotin/avidin Separation Systems

Any ligand/receptor model can be used to purify a double-stranded polynucleotide lacking a base pair mismatch-, an insertion/deletion loop and/or a nucleotide gap or gaps. For example, a biotin can be attached to a base pair mismatch-, an insertion/deletion loop- and/or a nucleotide gap binding polypeptide, or, it can be part of a fusion protein comprising a base pair mismatch-, an insertion/deletion loop- and/or a nucleotide gap-binding polypeptide. The biotin-binding avidin is typically immobilized, e.g., onto a bead, a magnetic material, a column, a gel and the like. The bead can be magnetized. See, e.g., the U.S. Patents noted above for making and using magnetic particles in purification techniques, and, describing various biotin-avidin binding systems and methods for making and using them, U.S. Pat. Nos. 6,287,792; 6,277,609; 6,214,974; 6,022,688; 5,484,701; 5,432,067; 5,374,516.

Generating and Manipulating Nucleic Acids

The invention provides methods for purifying double-stranded polynucleotides lacking base pair mismatches, insertion/deletion loops and/or a nucleotide gap or gaps. Nucleic acids purified by the methods of the invention can be amplified, cloned, sequence or further manipulated, e.g., their sequences can be further changed by SLR, GSSM and the like. The polypeptides used in the methods of the invention can be expressed recombinantly, synthesized or isolated from natural sources. These and other nucleic acids needed to make and use the invention can be isolated from a cell, recombinantly generated or made synthetically. The sequences can be isolated by, e.g., cloning and expression of cDNA libraries, amplification of message or genomic DNA by PCR, and the like. In practicing the methods of the invention, genes can be modified by manipulating a template nucleic acid, as described herein. The invention can be practiced in conjunction with any method or protocol or device known in the art, which are well described in the scientific and patent literature.

General Techniques

The nucleic acids used to practice this invention, whether RNA, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., subcloning, ligations, labeling probes (e.g., random-primer labeling using Klenow polymerase, nick translation, amplification), sequencing, hybridization and the like are well described in the scientific and patent literature, see, e.g., Sambrook, ed., Molecular Cloning: A Laboratory Manual (2ND ED.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridazation with Nucleic Acid Probes, Part I. Theory and Nucleic Acid Preparation, Tijssen, ed. Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Amplification of Nucleic Acids

In practicing the methods of the invention, nucleic acids can be generated and reproduced by, e.g., amplification reactions. Amplification reactions can also be used to join together nucleic acids to generate fusion protein coding sequences. Amplification reactions can also be used to clone sequences into vectors. Amplification reactions can also be used to quantify the amount of nucleic acid in a sample, label the nucleic acid (e.g., to apply it to an array or a blot), detect the nucleic acid, or quantify the amount of a specific nucleic acid in a sample. Message isolated from a cell or a cDNA library are amplified. The skilled artisan can select and design suitable oligonucleotide amplification primers. Amplification methods are also well known in the art, and include, e.g., polymerase chain reaction, PCR (see, e.g., PCR Protocols, A Guide to Methods and Applications, ed. Innis, Academic Press, N.Y. (1990) and PCR Strategies (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

Compositions and Methods for Making Polynucleotides by Iterative Assembly of Codon Building Blocks

The invention provides compositions and methods for making polynucleotides by iterative assembly of codon building blocks. The invention provides libraries of synthetic or recombinant oligonucleotides comprising multicodons (e.g., dicodons, tricodons, tetracodons and the like). The libraries comprise oligonucleotides comprising restriction endonuclease restriction sites, e.g., Type-IIS restriction endonuclease restriction sites, wherein the restriction endonuclease cuts at a fixed position outside of the recognition sequence to generate a single stranded overhang. In one aspect, the multicodon (e.g., dicodon) is flanked on both ends by a restriction endonuclease restriction site, e.g., Type-IIS restriction endonuclease restriction sites.

The invention also provides methods for generating any nucleic acid sequence, such as synthetic genes, antisense constructs, self-splicing introns or transcripts (e.g., ribozymes) and polypeptide coding sequences. The polynucleotide construction methods comprise use of libraries of pre-made oligonucleotide building blocks and Type-IIS restriction endonucleases. Type-IIS restriction endonucleases, upon digestion of an oligonucleotide library member, can generate a three, two or a one base single-stranded overhang. Type-IIS restriction endonucleases can include, e.g., SapI, EarI, BseRI, BsgI, BpmI, N.AlwI, N.BstNBI, BcgI, BsaXI or BspCNI or an isochizomer thereof.

In one aspect, the synthesis starts at a solid support, e.g., a bead, such as a magnetic bead, or a capillary, such as a GIGAMATRIX™, to which is immobilized a “starter” oligonucleotide fragment. In one aspect, a library of “elongation fragments” is used to build the nucleic acid sequence codon by codon. Where the “elongation fragments” comprise dicodons, the library has a total of all possible hexameric dicodon sequences, or 4096 “elongation fragment oligonucleotides.” Each “elongation fragment” is “embedded in” or flanked by Type-IIS restriction endonuclease recognition sites. Class IIS restriction endonucleases have specific recognition sequences and cut at a fixed distance outside the recognition site. Digestion produces compatible overhangs. Newly added fragments can be used in molar excess as compared to the immobilized oligonucleotide, or growing polynucleotide. The molar excess saturates free ends and drives the ligation to completion. Unbound material is washed away. The remaining 5′ overhangs can be filled in with Klenow DNA polymerase to block them from further elongation in a later cycle. Joined fragments can be ligated enzymatically. The process can be repeated, adding at least one codon in each cycle. The process can be iteratively repeated to produce a polynucleotide of any length. The synthesis can be started simultaneously at multiple points within the gene. Synthesized partial genes can be then released from the solid support, e.g., by a second set of restriction sites in the flanking regions and linked to form a desired full-length product, e.g., a polypeptide coding sequence, a transcript with or without 5′ and 3′ non-coding regions, a transcriptional control region, a gene.

In the methods of the invention, the same set of starter and elongation oligonucleotide fragments can be used for every synthesis. The methods of the invention of the invention can generate polynucleotides with very low error frequencies. The oligonucleotide building blocks, including the immobilized “starter” and the “elongation” oligonucleotides can be prepared from plasmid DNA as restriction fragments, or, they can be generated by nucleic acid amplification (e.g., PCR).

An exemplary polynucleotide synthetic scheme of the invention uses a library of pre-made building blocks to generate any given DNA sequence. The library can include all possible di-codon combinations, at total of 4096 clones to be used with 61 “starter” linker oligonucleotide fragments. As described in Example 1, below, in one aspect, each di-codon containing oligonucleotide “block” is cloned, sequence verified, PCR amplified or prepped from a restriction digest, and pre-cut (pre-digested) with a Type-IIS restriction endonuclease.

Building genes from oligonucleotides using the methods and libraries of the invention can eliminate the requirement of a “parental” or a template DNA. Using a codon by codon addition strategy allows custom design of nucleic acid sequences, including genes, antisense coding sequences, polypeptide coding sequences and others without the need for a “parental” or a template DNA. The methods and libraries of the invention can be used to design synthetic nucleic acids such that codon usage towards one or more specific expression hosts is optimized. Restriction sites can be designed according to individual cloning needs. The methods and libraries of the invention can be used to design and incorporate custom transcriptional regulatory elements linked to a coding sequence to achieve a desired level of expression or a cell-specific expression pattern. The compositions and methods of the invention can be used in conjunction with any other method, including methods using “parental” or a template DNA.

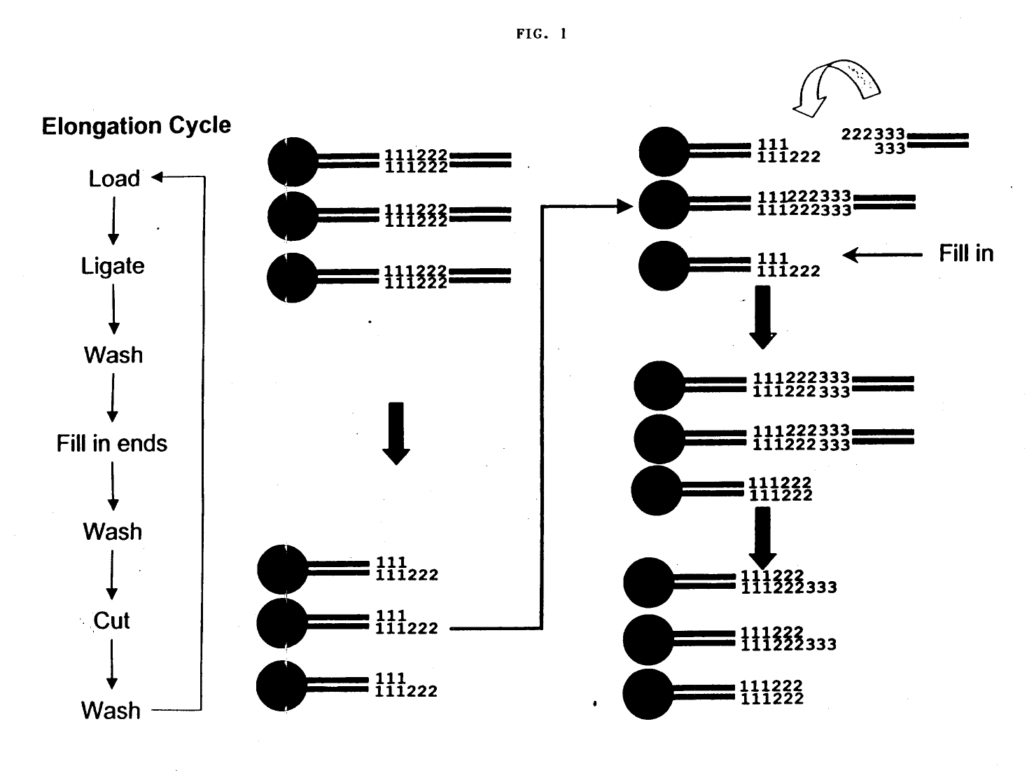

See FIG. 1 for a summary of this exemplary iterative codon by codon gene building protocol. In one aspect, a target DNA sequence is synthesized on a solid support (e.g., a bead or a capillary). As noted in FIG. 1, first a “starter” fragment containing at least a first codon is immobilized to the support. The “starter” oligonucleotide can be immobilized by a “hook” already on the support, e.g., the bead. In the next step, an “elongation fragment” comprising a multicodon (at least two codons, or a dicodon) is added. In this example the first “elongation fragment” comprises the first two codons. However, in other aspects of the invention, the “starter” fragments can comprise at least one codon. The joined ends are ligated. The cycle is completed after cutting with a restriction enzyme to generate a 5′ overhang. In this exemplary method, the restriction enzyme cuts in codon two such that the cycle adds one codon in each cycle.

In another aspect, because palindromic sequences may result in self-ligation of the fragments the 5′ overhangs can be filled in and converted to blunt ends using Klenow DNA polymerase to block them from annealing in later elongation cycles.

The building block oligonucleotide libraries of the invention can be prepared in vectors, thus, the building block oligonucleotide libraries of the invention can comprise a cloning vehicle, such as a vector. In the preparation of a library of the invention the choice of the vector and host strain may be important that the vector not contain restriction sites used in the preparation of the “building blocks.” A strain that produces unmodified DNA may need to be used because some of the class IIS restriction enzymes are sensitive to methylation. The “building blocks” can be prepared in a variety of ways, e.g., as restriction fragments, by high-fidelity PCR amplification, by synthetic chemistry.

In one aspect, these methods are performed as an automated, high throughput system. Supporting software can be used, e.g., for archiving and/or retrieval of sequenced clones, identifying the necessary building blocks in an array of clones or in a library for a given nucleic acid sequence. Any software system can be used, e.g., variations of DNACARPENTER™ software, Diversa Corporation, San Diego, Calif. Any robots system can be used for the automated, high throughput system.

Definitions

Unless defined otherwise, all technical and scientific terms used herein have the meaning commonly understood by a person skilled in the art to which this invention belongs. As used herein, the following terms have the meanings ascribed to them unless specified otherwise.

The terms “Type-IIS enzyme” or “Type-IIS restriction endonuclease” include all restriction endonucleases and all isochizomers having an asymmetric recognition sequence that cut at a fixed position outside of the recognition sequence at one strand or both strands, either 3′ or 5′ or on both sides of the recognition sequence. Type IIS enzymes can recognize asymmetric base sequences and cleave DNA at a specified position up to 20 or more base pairs outside of the recognition site. In one aspect, they can cleave a few nucleotides away from the recognition sequence (see, e.g., Bath (2001) Biol. Chem. November 29; epub). Exemplary restriction endonucleases that cut on both sides include BcgI (see, e.g., Kong (1998) J. Mol. Biol. 279:823-32), BsaXI and BspCNI. Exemplary restriction endonucleases that generate a three base single-stranded overhang include EarI and SapI. Exemplary restriction endonucleases that generate a two base single-stranded overhang include BseRI, BsgI (see, e.g., Ariazi (1996) Biotechniques 20:446-448, 450-451) and BpmI. Exemplary restriction endonucleases that generate a one base single-stranded overhang include BmrI; EciI, HphI, MboII (see, e.g., Soundararajan (2001) J. Biol. Chem. October 17; epub) and MnII. Exemplary restriction endonucleases that cut only one strand (“nicking enzymes”) include N.AlwI and N.BstNBI. Any Type IIS enzyme can be used in the methods of the invention, including, e.g., BspMI (see, e.g., Gormley (2001) J. Biol. Chem. November 29; epub) and BcefI (see, e.g., Venetianer (1988) Nucleic Acids Res. 16:3053-3060).

“EarI” includes all Type-IIS restriction endonucleases which recognize 5′-CTCTTC-3′ and all isochizomers and restriction endonucleases having the same recognition sequence and base cleaving pattern (isochizomers have the same the specificity of the prototype restriction endonuclease). EarI was first isolated from an Enterobacter aerogenes. See, e.g., Polisson (1988) Nucleic Acids Res. 16:9872.

“SapI” includes all Type-IIS restriction endonucleases which recognize the non-palindromic 7-base recognition sequence (GCTCTTC) and all isochizomers and restriction endonucleases having the same recognition sequence and base-cleaving pattern. See, e.g., Xu (1998) Mol. Gen. Genet. 260:226-231.

The term “saturation mutagenesis” or “GSSM” includes a method that uses degenerate oligonucleotide primers to introduce point mutations into a polynucleotide, as described in detail herein.

The term “optimized directed evolution system” or “optimized directed evolution” includes a method for reassembling fragments of related nucleic acid sequences, e.g., related genes, and explained in detail herein.

The term “synthetic ligation reassembly” or “SLR” includes a method of ligating oligonucleotide fragments in a non-stochastic fashion, and explained in detail herein.

The terms “nucleic acid” and “polynucleotide” as used herein include deoxyribonucleotides or ribonucleotides in either single- or double-stranded form. The terms encompass all nucleic acids, e.g., oligonucleotides, and modifications analogues of natural nucleotides, e.g., nucleic acids with modified internucleoside linkages. The terms also encompass nucleic-acid-like structures with synthetic backbones. Synthetic backbone analogues include, e.g., phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs can contain non-ionic backbones, such as N-(2-aminoethyl) glycine units, see, e.g., U.S. Pat. No. 5,871,902. Phosphorothioate linkages are described, e.g., in WO 97/03211; WO 96/39154; Mata (1997) Toxicol. Appl. Pharmacol. 144:189-197. Other synthetic backbones include methyl-phosphonate linkages or alternating methylphosphonate and phosphodiester linkages (Strauss-Soukup (1997) Biochemistry 36:8692-8698), and benzylphosphonate linkages (Samstag (1996) Antisense Nucleic Acid Drug Dev 6:153-156). Modified internucleoside linkages that are resistant to nucleases are described, e.g., in U.S. Pat. No. 5,817,781. The term nucleic acid and polynucleotide can be used interchangeably with the terms gene, cDNA, mRNA, probe and amplification product.

Generating and Manipulating Nucleic Acids

The invention provides libraries of nucleic acids (oligonucleotides and polynucleotides) and methods of making and using these libraries. The invention also provides methods for making nucleic acids using a codon by codon building technique and methods for further manipulation of these nucleic acids, including cloning, sequencing and expressing them. Nucleic acids, including individual bases, codons, oligos, and the like, needed to make and use the invention can be isolated from a cell, recombinantly generated or made synthetically. Sequences can be isolated by, e.g., cloning and expression of cDNA libraries, amplification of message or genomic DNA by PCR, and the like. The invention can be practiced in conjunction with any method or protocol or device known in the art, which are well described in the scientific and patent literature.

General Techniques

Nucleic acids (including individual bases, codons, oligos, and the like) used to practice this invention, whether RNA, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids (including individual bases, codons, oligos, and the like) can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., subcloning, ligations, labeling probes (e.g., random-primer labeling using Klenow polymerase, nick translation, amplification), sequencing, hybridization and the like are well described in the scientific and patent literature, see, e.g., Sambrook, ed., Molecular Cloning: A Laboratory Manual (2nd ed.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridization with Nucleic Acid Probes, Part I. Theory and Nucleic Acid Preparation, Tijssen, ed. Elsevier, N.Y. (1993).

Nucleic acids, oligonucleotides, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

A variety of enzymes and buffers can be used in the methods and systems of the invention, including restriction endonucleases (e.g., type IIS endonucleases), DNA ligases, Klenow DNA polymerases and the like. Buffers and reactions conditions, e.g., incubation times, temperatures, amount of enzyme and nucleic acid used for each step, can be optimized for each step by routine methods.

Amplification of Nucleic Acids

In practicing the methods of the invention, nucleic acids and oligonucleotides can be manipulated, sequenced, cloned, reproduced and the like by amplification reactions. Amplification reactions can be used to splice together nucleic acids or oligonucleotides or clone them into vectors. Amplification reactions can also be used to quantify the amount of nucleic acid in a sample, label the nucleic acid (e.g., to apply it to an array or a blot), detect the nucleic acid, or quantify the amount of a specific nucleic acid in a sample. The skilled artisan can select and design suitable oligonucleotide amplification primers. Amplification methods are also well known in the art, and include, e.g., polymerase chain reaction, PCR (see, e.g., PCR Protocols, A Guide to Methods And Applications, ed. Innis, Academic Press, N.Y. (1990) and PCR Strategies (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

Substrate Surfaces

The invention provides a method for building a polynucleotide by iterative assembly of multicodon, e.g., dicodon, building blocks comprising providing a substrate surface and immobilizing an oligonucleotide to the substrate surface. Any substrate surface can be used to practice the invention. For example, substrate surfaces can be of rigid, semi-rigid or flexible material. Substrate surfaces can be flat or planar, be shaped as wells, raised regions, etched trenches, pores, beads, filaments, or the like. Substrate surfaces can be of any material upon which a “capture probe” can be directly or indirectly bound. For example, suitable materials can include paper, glass (see, e.g., U.S. Pat. No. 5,843,767), ceramics, quartz or other crystalline substrates (e.g. gallium arsenide), metals, metalloids, polacryloylmorpholide, various plastics and plastic copolymers, Nylon™, Teflon™, polyethylene, polypropylene, poly(4-methylbutene), polystyrene, polystyrene/latex, polymethacrylate, poly(ethylene terephthalate), rayon, nylon, poly(vinyl butyrate), polyvinylidene difluoride (PVDF) (see, e.g., U.S. Pat. No. 6,024,872), silicones (see, e.g., U.S. Pat. No. 6,096,817), polyformaldehyde (see, e.g., U.S. Pat. Nos. 4,355,153; 4,652,613), cellulose (see, e.g., U.S. Pat. No. 5,068,269), cellulose acetate (see, e.g., U.S. Pat. No. 6,048,457), nitrocellulose, various membranes and gels (e.g., silica aerogels, see, e.g., U.S. Pat. No. 5,795,557), paramagnetic or superparamagnetic microparticles (see, e.g., U.S. Pat. No. 5,939,261) and the like. Silane (e.g., mono- and dihydroxyalkylsilanes, aminoalkyltrialkoxysilanes, 3-aminopropyl-triethoxysilane, 3-aminopropyltrimethoxysilane) can provide a hydroxyl functional group for reaction with an amine functional group.

In one aspect, the invention provides a set of beads, e.g., magnetic beads (including, e.g., paramagnetic or superparamagnetic microparticles), comprising 61 “starter” oligonucleotides, one bead for each possible amino acid coding triplet. In another aspect, the invention provides a system comprising these 61 “starter” oligonucleotides and 4⁶ or 1096 possible hexameric dicodon oligonucleotides. As discussed above, these dicodon oligonucleotides are “embedded” in, or flanked by, a framework of endonuclcase recognition sites, e.g., class IIS restriction sites. The 61 “starter” oligonucleotides can be immobilized onto modalities other than beads, e.g., wells, strands, capillary tubes (see below, e.g., capillary arrays, such as the GIGAMATRIX™), troughs and the like.

Capillary Arrays

Capillary arrays, such as the GIGAMATRIX™, Diversa Corporation, San Diego, Calif., can be used as a substrate surface. Capillary arrays provide another system for immobilizing and building nucleic acids using the methods of the invention. Once constructed, the immobilized newly constructed polynucleotides can be screened and expressed within the capillary array. A plurality of capillaries can be formed into an array of adjacent capillaries, wherein each capillary comprises at least one wall defining a lumen for retaining an oligonucleotide. The apparatus can further include interstitial material disposed between adjacent capillaries in the array, and one or more reference indicia formed within of the interstitial material. A capillary for screening a sample, wherein the capillary is adapted for being bound in an array of capillaries, can include a first wall defining a lumen for retaining the sample, and a second wall formed of a filtering material, for filtering excitation energy provided to the lumen to excite the sample. See, e.g., WO0138583.

For example, a nucleic acid, e.g., a codon-comprising library member, can be introduced into a first component into at least a portion of a capillary of a capillary array. Each capillary of the capillary array can comprise at least one wall defining a lumen for retaining the first component, and introducing an air bubble into the capillary behind the first component. A second component (e.g., a different buffer, an endonuclease enzyme, a codon-comprising library member) can be introduced into the capillary, wherein the second component is separated from the first component by the air bubble. A sample (e.g., comprising a codon-comprising library member) can be introduced as a first liquid labeled with a detectable particle into a capillary of a capillary array, wherein each capillary of the capillary array comprises at least one wall defining a lumen for retaining the first liquid and the detectable particle, and wherein the at least one wall is coated with a binding material for binding the detectable particle to the at least one wall. The method can further include removing the first liquid from the capillary tube, wherein the bound detectable particle is maintained within the capillary, and introducing a second liquid into the capillary tube.

The capillary array can include a plurality of individual capillaries comprising at least one outer wall defining a lumen. The outer wall of the capillary can be one or more walls fused together. Similarly, the wall can define a lumen that is cylindrical, square, hexagonal or any other geometric shape so long as the walls form a lumen for retention of a liquid or sample. The capillaries of the capillary array can be held together in close proximity to form a planar structure. The capillaries can be bound together, by being fused (e.g., where the capillaries are made of glass), glued, bonded, or clamped side-by-side. The capillary array can be formed of any number of individual capillaries, for example, a range from 100 to 4,000,000 capillaries. A capillary array can form a microtiter plate having about 100,000 or more individual capillaries bound together.

Modification of Nucleic Acids

The nucleic acids generated by the methods of the invention can be altered by any means, including saturation mutagenesis, an optimized directed evolution system, synthetic ligation reassembly, or a combination thereof, as described herein. Random or stochastic methods, or, non-stochastic, or “directed evolution,” methods can be used. Further, as discussed above, the nucleic acids generated by the methods of the invention can be purified by the methods described herein, e.g., the methods for purifying double-stranded polynucleotides lacking base pair mismatches, insertion/deletion loops and/or a nucleotide gap or gaps as described herein. The nucleic acids generated by the methods of the invention can be altered by a method comprising gene site saturated mutagenesis (GSSM), error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, synthetic ligation reassembly (SLR) and a combination thereof. The nucleic acids generated by the methods of the invention can be altered by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof.

Methods for random mutation of genes are well known in the art, see, e.g., U.S. Pat. No. 5,830,696. Mutagens include, e.g., ultraviolet light or gamma irradiation, or a chemical mutagen, e.g., mitomycin, nitrous acid, photoactivated psoralens, alone or in combination, to induce DNA breaks amenable to repair by recombination. Other chemical mutagens include, for example, sodium bisulfite, nitrous acid, hydroxylamine, hydrazine or formic acid. Other mutagens are analogues of nucleotide precursors, e.g., nitrosoguanidine, 5-bromouracil, 2-aminopurine, or acridine. These agents can be added to a PCR reaction in place of the nucleotide precursor thereby mutating the sequence. Intercalating agents such as proflavine, acriflavine, quinacrine and the like can also be used.

Techniques in molecular biology can be used, e.g., random PCR mutagenesis, see, e.g., Rice (1992) Proc. Natl. Acad. Sci. USA 89:5467-5471; or, combinatorial multiple cassette mutagenesis, see, e.g., Crameri (1995) Biotechniques 18:194-196. Alternatively, nucleic acids, e.g., genes, can be reassembled after random, or “stochastic,” fragmentation, see, e.g., U.S. Pat. Nos. 6,291,242; 6,287,862; 6,287,861; 5,955,358; 5,830,721; 5,824,514; 5,811,238; 5,605,793. Polypeptides encoded by isolated and/or modified nucleic acids can be screened for an activity before their reinsertion into the cell by, e.g., using a capillary array platform. See, e.g., U.S. Pat. Nos. 6,280,926; 5,939,250.

Saturation Mutagenesis, or, GSSM

In one aspect of the invention, non-stochastic gene modification, a “directed evolution process,” can be used to modify nucleic acids generated by the methods of the invention. Variations of this method have been termed “gene site-saturation mutagenesis,”“site-saturation mutagenesis,” “saturation mutagenesis” or simply “GSSM.” It can be used in combination with other mutagenization processes. See, e.g., U.S. Pat. Nos. 6,171,820; 6,238,884. In one aspect, GSSM comprises providing a template polynucleotide and a plurality of oligonucleotides, wherein each oligonucleotide comprises a sequence homologous to the template polynucleotide, thereby targeting a specific sequence of the template polynucleotide, and a sequence that is a variant of the homologous gene; generating progeny polynucleotides comprising non-stochastic sequence variations by replicating the template polynucleotide with the oligonucleotides, thereby generating polynucleotides comprising homologous gene sequence variations.

In another aspect, site-saturation mutagenesis can be used together with another stochastic or non-stochastic means to vary sequence, e.g., synthetic ligation reassembly (see below), shuffling, chimerization, recombination and other mutagenizing processes and mutagenizing agents. This invention provides for the use of any mutagenizing process(es), including saturation mutagenesis, in an iterative manner.

Synthetic Ligation Reassembly (SLR)

Another non-stochastic gene modification, a “directed evolution process,” that can be can be used to modify nucleic acids generated by the methods of the invention has been termed “synthetic ligation reassembly,” or simply “SLR.” SLR is a method of ligating oligonucleotide fragments together non-stochastically. This method differs from stochastic oligonucleotide shuffling in that the nucleic acid building blocks are not shuffled, concatenated or chimerized randomly, but rather are assembled non-stochastically. See, e.g., U.S. patent application Ser. No. (USSN) 09/332,835 entitled “Synthetic Ligation Reassembly in Directed Evolution” and filed on Jun. 14, 1999 (“U.S. Ser. No. 09/332,835”). In one aspect, SLR comprises the following steps: (a) providing a template polynucleotide, wherein the template polynucleotide comprises sequence encoding a homologous gene; (b) providing a plurality of building block polynucleotides, wherein the building block polynucleotides are designed to cross-over reassemble with the template polynucleotide at a predetermined sequence, and a building block polynucleotide comprises a sequence that is a variant of the homologous gene and a sequence homologous to the template polynucleotide flanking the variant sequence; (c) combining a building block polynucleotide with a template polynucleotide such that the building block polynucleotide cross-over reassembles with the template polynucleotide to generate polynucleotides comprising homologous gene sequence variations.

SLR does not depend on the presence of high levels of homology between polynucleotides to be rearranged. Thus, this method can be used to non-stochastically generate libraries (or sets) of progeny molecules comprised of over 10¹⁰⁰ different chimeras. SLR can be used to generate libraries comprised of over 10¹⁰⁰⁰ different progeny chimeras. Thus, aspects of the present invention include non-stochastic methods of producing a set of finalized chimeric nucleic acid molecule shaving an overall assembly order that is chosen by design. This method includes the steps of generating by design a plurality of specific nucleic acid building blocks having serviceable mutually compatible ligatable ends, and assembling these nucleic acid building blocks, such that a designed overall assembly order is achieved.

Optimized Directed Evolution System

Nucleic acids generated by the methods of the invention can also be modified by a method comprising an optimized directed evolution system. Optimized directed evolution is directed to the use of repeated cycles of reductive reassortment, recombination and selection that allow for the directed molecular evolution of nucleic acids through recombination. Optimized directed evolution allows generation of a large population of evolved chimeric sequences, wherein the generated population is significantly enriched for sequences that have a predetermined number of crossover events. A crossover event is a point in a chimeric sequence where a shift in sequence occurs from one parental variant to another parental variant. Such a point is normally at the juncture of where oligonucleotides from two parents are ligated together to form a single sequence. This method allows calculation of the correct concentrations of oligonucleotide sequences so that the final chimeric population of sequences is enriched for the chosen number of crossover events. This provides more control over choosing chimeric variants having a predetermined number of crossover events.

In addition, this method provides a convenient means for exploring a tremendous amount of the possible protein variant space. By using optimized directed evolution system, a population of nucleic acid molecules can be enriched for those variants that have a particular number of crossover events. One method for creating a chimeric progeny polynucleotide sequence is to create oligonucleotides corresponding to fragments or portions of each parental sequence. Each oligonucleotide can include a unique region of overlap so that mixing the oligonucleotides together results in a new variant that has each oligonucleotide fragment assembled in the correct order. Additional information can also be found in WO0077262; WO0058517; WO0046344.

Chimeric Antigen Binding Molecules and Methods for Making and Using Them

The invention provides novel chimeric antigen binding polypeptides, nucleic acids encoding them and methods for making and using them. This invention also provides methods for further modifying these chimeric antigen binding polypeptides by altering the nucleic acids that encode them by saturation mutagenesis, an optimized directed evolution system, synthetic ligation reassembly, or a combination thereof. These modifications can focus on such as antigen binding sites or specific domains or fragments of antibodies, e.g., variable or heavy domains, Fab or Fc domains or CDRs.

The invention also provides libraries of chimeric antigen binding polypeptides encoded by the nucleic acid libraries of the invention and generated by the methods of the invention. These antigen binding polypeptides can be analyzed using any liquid or solid state screening method, e.g., phage display, ribosome display, using capillary array platforms, e.g., GIGAMATRIX™, and the like.

The chimeric antigen binding polypeptides generated by the methods of the invention can be used in vitro, e.g., to isolate, measure amounts of, or identify antigens or in vivo, e.g., to treat or diagnose various diseases and conditions, or to modulate, stimulate or attenuate an immune response. The antigen binding polypeptides of the invention can be manipulated to be catalytic antibodies, see, e.g., U.S. Pat. Nos. 6,326,179; 5,439,812; 5,302,516; 5,187,086; 5,126,258.

This invention also pertains to the field of vaccines. The libraries and methods of the invention provide manipulated antigen binding polypeptides, including polypeptide antibodies and genetic vaccines comprising nucleic acids. Specific antigen binding polypeptides can be selected for optimization by the methods of the invention for a particular vaccination goal. Antibodies can be designed for administration to generate passive immunity. Nucleic acids encoding these antigen binding polypeptides can be used as genetic vaccines. In one aspect, this invention provides methods for improving the efficacy of genetic vaccines by providing antigen binding polypeptides that facilitate targeting of a genetic vaccine to a particular tissue or cell type of interest.

This invention pertains to the field biologic therapeutics by providing polypeptides comprising antigen binding sites, such as antibodies, with modified (e.g., increased or decreased) affinity for antigen. For example, the methods of the invention provide antibodies of altered or enhanced affinities for an antigen for use, e.g., in immunotherapeutics or diagnostics. The antibodies generated by the methods of the invention can be administered therapeutically to slow the growth of or kill cells, such as cancer cells, or, to stimulate cell division, e.g., for enhancing an immune response or for tissue regeneration, or, to alter any biological mechanism or response. For example, administration of antibodies that bind to immune effector or regulatory cells, or to lymphokines or cytokines, can alter, e.g., upregulate, stimulate or attenuate, a humoral or a cellular immune response. This invention also can be used to develop efficient immune responses against a broad range of antigens.

This invention pertains to the field of modulation of immune responses by providing chimeric antigen binding polypeptides specific for molecules that are involved in the stimulation and regulation of the immune response, including, e.g., Fc receptors, surface expressed (membrane bound) immunoglobulins, T cell receptors or Class I and Class II major histocompatibility (MHC) molecules. For example, by modulating expression of one or more these molecules the methods of the invention can modulate autoreactive TCR reactions, generate an abated or attenuated immune response to a self antigen or generate an enhanced immune response, e.g., to a pathogen.

This invention also relates to the field of protein engineering. The invention uses directed evolution methods for modifying polynucleotides encoding the chimeric antigen binding polypeptides of the invention. Methods of mutagenesis are used to generate novel polynucleotides encoding chimeric antigen binding polypeptides that are altered, or “improved.” These methods include non-stochastic polynucleotide chimerization and non-stochastic site-directed point mutagenesis.

In one aspect, this invention relates to a method of generating a progeny library, or set, of chimeric antigen binding polynucleotide(s) by means that are synthetic and non-stochastic. The design of the progeny antigen binding polynucleotide(s) is derived by analysis of a parental set of antigen binding polynucleotides and/or of the polypeptides correspondingly encoded by the parental polynucleotides. In another aspect, this invention relates to a method of performing site-directed mutagenesis using means that are exhaustive, systematic, and non-stochastic.

This invention also includes selecting from among a generated set of progeny chimeric antigen binding molecules a subset comprised of particularly desirable species, including by a process termed end-selection, which subset may then be screened further. This invention also includes screening a set of antigen binding polynucleotides. The antigen binding polypeptides can be re-designed to have a useful property, such as having an increased affinity (e.g., “affinity enrichment”) or decreased affinity for an antigen, or gaining or changing its ability to act as an enzyme.

The methods of the invention provide for “affinity enrichment” of a chimeric antibody or an antigen binding site. Antibody constant regions (e.g., Fc domains) can also be “affinity enriched” for their ability to specifically bind to an Fc receptor or a complement polypeptide. Very large sets, or libraries, of variant antibodies, including, e.g., CDRs, Fabs, Fcs, and single-chain antibodies, can be generated and screened for binding to ligand (e.g., antigen, complement, receptor, and the like). In one aspect, the variant polynucleotide is isolated and further manipulated by a method described herein, e.g., shuffled to recombine combinatorially the amino acid sequence of the selected polypeptides, peptide(s) or predetermined portions thereof. Thus, antibodies, antigen binding sites, Fc domains, and the like can be generated having a desired binding affinity for a molecule. The peptide or antibody can then be synthesized in bulk by conventional means for any suitable use (e.g., as a therapeutic pharmaceutical, a diagnostic agent, or as an in vitro reagent).

Definitions

Unless defined otherwise, all technical and scientific terms used herein have the meaning commonly understood by a person skilled in the art to which this invention belongs. As used herein, the following terms have the meanings ascribed to them unless specified otherwise.

The term “saturation mutagenesis” or “GSSM” includes a method that uses degenerate oligonucleotide primers to introduce point mutations into a polynucleotide, as described in detail, below. In one aspect, the methods of the invention further comprise non-stochastic modification of all or a part of the sequence of a chimeric antibody coding sequence of the invention by “saturation mutagenesis” or “GSSM.”

The term “optimized directed evolution system” or “optimized directed evolution” includes a method for reassembling fragments of related nucleic acid sequences, e.g., related genes, and explained in detail, below. In one aspect, the methods of the invention further comprise non-stochastic modification of all or a part of the sequence of a chimeric antibody coding sequence of the invention by “optimized directed evolution system.”

The term “synthetic ligation reassembly” or “SLR” includes a method of ligating oligonucleotide fragments in a non-stochastic fashion, and explained in detail, below. In one aspect, the methods of the invention further comprise non-stochastic modification of all or a part of the sequence of a chimeric antibody coding sequence of the invention by “synthetic ligation reassembly” or “SLR.”

The term “antibody” includes a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y. (1993); Wilson (1994) J. Immunol. Methods 175:267-73; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

Generating and Manipulating Nucleic Acids

The invention provides libraries of chimeric nucleic acids encoding a plurality of chimeric antigen binding polypeptides and methods for making these libraries. Making these libraries comprises providing nucleic acids encoding lambda light chain variable region polypeptide domains (Vλ), kappa light chain variable region polypeptide domains (Vκ), J region polypeptide domains (VJ), lambda light chain constant region polypeptide domains (Cλ), kappa light chain constant region polypeptide domains (Cκ), antibody heavy chain variable region polypeptide domains (VH), D region polypeptide domains (VD), J region polypeptide domains (VJ) and heavy chain constant region polypeptide domains (CH).

These and other nucleic acids needed to make and use the invention can be isolated from a cell, recombinantly generated or made synthetically. The sequences can be isolated by, e.g., cloning and expression of cDNA libraries, amplification of message or genomic DNA by PCR, and the like. In practicing the methods of the invention, homologous genes can be modified by manipulating a template nucleic acid, as described herein. The invention can be practiced in conjunction with any method or protocol or device known in the art, which are well described in the scientific and patent literature.

General Techniques

The nucleic acids used to practice this invention, whether RNA, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., subcloning, ligations, labeling probes (e.g., random-primer labeling using Klenow polymerase, nick translation, amplification), sequencing, hybridization and the like are well described in the scientific and patent literature, see, e.g., Sambrook, ed., Molecular Cloning: A Laboratory Manual (2nd ed.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridization with Nucleic Acid Probes, Part I. Theory and Nucleic Acid Preparation, Tijssen, ed. Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

Amplification of Nucleic Acids

In practicing the methods of the invention, nucleic acids encoding lambda light chain variable region polypeptide domains (Vλ), kappa light chain variable region polypeptide domains (Vκ), J region polypeptide domains (VJ), lambda light chain constant region polypeptide domains (Cλ), kappa light chain constant region polypeptide domains (Cκ), antibody heavy chain variable region polypeptide domains (VH), D region polypeptide domains (VD), J region polypeptide domains (VJ) and heavy chain constant region polypeptide domains (CH) can be generated and reproduced by, e.g., amplification reactions. Amplification reactions can also be used to join together these domains or splice the chimeric nucleic acids of the invention into vectors. Amplification reactions can also be used to quantify the amount of nucleic acid in a sample, label the nucleic acid (e.g., to apply it to an array or a blot), detect the nucleic acid, or quantify the amount of a specific nucleic acid in a sample. In one aspect of the invention, message isolated from a cell or a cDNA library are amplified. The skilled artisan can select and design suitable oligonucleotide amplification primers. Amplification methods are also well known in the art, and include, e.g., polymerase chain reaction, PCR (see, e.g., PCR PROTOCOLS, A GUIDE to METHODS and APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

Immunoglobulin Coding Sequences

The invention provides chimeric antigen binding polypeptides including lambda light chain variable region polypeptide domains (Vλ), kappa light chain variable region polypeptide domains (Vκ), J region polypeptide domains (VJ), lambda light chain constant region polypeptide domains (Cλ), kappa light chain constant region polypeptide domains (Cκ), antibody heavy chain variable region polypeptide domains (VH), D region polypeptide domains (VD), J region polypeptide domains (VJ) and heavy chain constant region polypeptide domains (CH) and the chimeric nucleic acids encoding them. These sequences can be modeled from, cloned or amplified from or directed isolated from any gene or message, including cDNA, sequence.

Any cell can be used to as a source of antigen binding polypeptide coding sequence, including lymphocytes, such as B cells. Rearranged or activated B cells or plasma cells in the circulation, a lymph node or the spleen can be used. Any vertebrate can be a cell source. The repertoire of rearranged genes can be biased for a pre-determined binding specificity. For example, an animal can be immunized prior to isolating rearranged B cells or plasma cells. This generates a repertoire enriched for genetic material producing a ligand binding polypeptide of high affinity.

Alternatively, nucleic acids encoding immunoglobulin sequences an be modeled after already characterized coding sequences, many of which are known and characterized in the art, as, e.g., Genbank sequences, or, for sequences or methods to isolate such sequences e.g., see U.S. Pat. Nos. 6,319,690; 6,291,161; 6,258,529; 6,214,984; 6,204,023; 6,068,840; 6,057,421; 5,891,438; 5,869,619; 5,861,499; 5,851,801; 5,821,123.

Modification of Nucleic Acids

In one aspect of the methods of the invention, chimeric antigen binding polypeptide coding sequences are modified to alter the properties of the polypeptides they encode. The nucleic acids can be altered by any means, including saturation mutagenesis, an optimized directed evolution system, synthetic ligation reassembly, or a combination thereof, as described herein. Random or stochastic methods, or, non-stochastic, or “directed evolution,” methods can be used. These nucleic acid modifying procedures can target specific domains, e.g., lambda light chain variable region polypeptide domains (Vλ), kappa light chain variable region polypeptide domains (Vκ), J region polypeptide domains (VJ), lambda light chain constant region polypeptide domains (Cλ), kappa light chain constant region polypeptide domains (Cκ), antibody heavy chain variable region polypeptide domains (VH), D region polypeptide domains (VD), J region polypeptide domains (VJ) or heavy chain constant region polypeptide domains (CH). They can also specifically regions encoding target antigen binding sites or CDRs.

Further, the nucleic acids encoding these antibodies can be purified by the methods described herein, e.g., the methods for purifying double-stranded polynucleotides lacking base pair mismatches, insertion/deletion loops and/or a nucleotide gap or gaps as described herein.

The nucleic acids encoding the chimeric antigen binding polypeptide coding sequences can be modified by a method comprising gene site saturated mutagenesis (GSSM), error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, synthetic ligation reassembly (SLR) and a combination thereof. The nucleic acids generated by the methods of the invention can be altered by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof.

Methods for random mutation of genes are well known in the art, see, e.g., U.S. Pat. No. 5,830,696. For example, mutagens can be used to randomly mutate a gene. Mutagens include, e.g., ultraviolet light or gamma irradiation, or a chemical mutagen, e.g., mitomycin, nitrous acid, photoactivated psoralens, alone or in combination, to induce DNA breaks amenable to repair by recombination. Other chemical mutagens include, for example, sodium bisulfite, nitrous acid, hydroxylamine, hydrazine or formic acid. Other mutagens are analogues of nucleotide precursors, e.g., nitrosoguanidine, 5-bromouracil, 2-aminopurine, or acridine. These agents can be added to a PCR reaction in place of the nucleotide precursor thereby mutating the sequence. Intercalating agents such as proflavine, acriflavine, quinacrine and the like can also be used.

Techniques in molecular biology can be used, e.g., random PCR mutagenesis, see, e.g., Rice (1992) Proc. Natl. Acad. Sci. USA 89:5467-5471; or, combinatorial multiple cassette mutagenesis, see, e.g., Crameri (1995) Biotechniques 18:194-196. Alternatively, nucleic acids, e.g., genes, can be reassembled after random, or “stochastic,” fragmentation, see, e.g., U.S. Pat. Nos. 6,291,242; 6,287,862; 6,287,861; 5,955,358; 5,830,721; 5,824,514; 5,811,238; 5,605,793. Polypeptides encoded by isolated and/or modified nucleic acids can be screened for an activity before their reinsertion into the cell by, e.g., using a capillary array platform. See, e.g., U.S. Pat. Nos. 6,280,926; 5,939,250.

Saturation Mutagenesis, or, GSSM

In one aspect of the invention, non-stochastic gene modification, a “directed evolution process,” can be used to modify chimeric antigen binding polypeptide coding sequences. Variations of this method have been termed “gene site-saturation mutagenesis,”“site-saturation mutagenesis,” “saturation mutagenesis” or simply “GSSM.” It can be used in combination with other mutagenization processes. See, e.g., U.S. Pat. Nos. 6,171,820; 6,238,884. In one aspect, GSSM comprises providing a template polynucleotide and a plurality of oligonucleotides, wherein each oligonucleotide comprises a sequence homologous to the template polynucleotide, thereby targeting a specific sequence of the template polynucleotide, and a sequence that is a variant of the homologous gene; generating progeny polynucleotides comprising non-stochastic sequence variations by replicating the template polynucleotide with the oligonucleotides, thereby generating polynucleotides comprising homologous gene sequence variations.

In one aspect, codon primers containing a degenerate N,N,G/T sequence are used to introduce point mutations into a polynucleotide, so as to generate a set of progeny polypeptides in which a full range of single amino acid substitutions is represented at each amino acid position, e.g., an amino acid residue in an enzyme active site or ligand binding site targeted to be modified. These oligonucleotides can comprise a contiguous first homologous sequence, a degenerate N,N,G/T sequence, and, optionally, a second homologous sequence. The downstream progeny translational products from the use of such oligonucleotides include all possible amino acid changes at each amino acid site along the polypeptide, because the degeneracy of the N,N,G/T sequence includes codons for all 20 amino acids.

In one aspect, one such degenerate oligonucleotide (comprised of, e.g., one degenerate N,N,G/T cassette) is used for subjecting each original codon in a parental polynucleotide template to a full range of codon substitutions. In another aspect, at least two degenerate cassettes are used—either in the same oligonucleotide or not, for subjecting at least two original codons in a parental polynucleotide template to a full range of codon substitutions. For example, more than one N,N,G/T sequence can be contained in one oligonucleotide to introduce amino acid mutations at more than one site. This plurality of N,N,G/T sequences can be directly contiguous, or seperated by one or more additional nucleotide sequence(s). In another aspect, oligonucleotides serviceable for introducing additions and deletions can be used either alone or in combination with the codons containing an N,N,G/T sequence, to introduce any combination or permutation of amino acid additions, deletions, and/or substitutions.

In one aspect, simultaneous mutagenesis of two or more contiguous amino acid positions is done using an oligonucleotide that contains contiguous N,N,G/T triplets, i.e. a degenerate (N,N,G/T)n sequence. In another aspect, degenerate cassettes having less degeneracy than the N,N,G/T sequence are used. For example, it may be desirable in some instances to use (e.g. in an oligonucleotide) a degenerate triplet sequence comprised of only one N, where said N can be in the first second or third position of the triplet. Any other bases including any combinations and permutations thereof can be used in the remaining two positions of the triplet. Alternatively, it may be desirable in some instances to use (e.g. in an oligo) a degenerate N,N,N triplet sequence.

In one aspect, use of degenerate triplets (e.g., N,N,G/T triplets) allows for systematic and easy generation of a full range of possible natural amino acids (for a total of 20 amino acids) into each and every amino acid position in a polypeptide (in alternative aspects, the methods also include generation of less than all possible substitutions per amino acid residue, or codon, position). For example, for a 100 amino acid polypeptide, 2000 distinct species (i.e. 20 possible amino acids per position×100 amino acid positions) can be generated. Through the use of an oligonucleotide or set of oligonucleotides containing a degenerate N,N,G/T triplet, 32 individual sequences can code for all 20 possible natural amino acids. Thus, in a reaction vessel in which a parental polynucleotide sequence is subjected to saturation mutagenesis using at least one such oligonucleotide, there are generated 32 distinct progeny polynucleotides encoding 20 distinct polypeptides. In contrast, the use of a non-degenerate oligonucleotide in site-directed mutagenesis leads to only one progeny polypeptide product per reaction vessel. Nondegenerate oligonucleotides can optionally be used in combination with degenerate primers disclosed; for example, nondegenerate oligonucleotides can be used to generate specific point mutations in a working polynucleotide. This provides one means to generate specific silent point mutations, point mutations leading to corresponding amino acid changes, and point mutations that cause the generation of stop codons and the corresponding expression of polypeptide fragments.

In one aspect, each saturation mutagenesis reaction vessel contains polynucleotides encoding at least 20 progeny polypeptide molecules such that all 20 natural amino acids are represented at the one specific amino acid position corresponding to the codon position mutagenized in the parental polynucleotide (other aspects use less than all 20 natural combinations). The 32-fold degenerate progeny polypeptides generated from each saturation mutagenesis reaction vessel can be subjected to clonal amplification (e.g. cloned into a suitable host, e.g., E. coli host, using, e.g., an expression vector) and subjected to expression screening. When an individual polypeptide is identified (e.g., by screening) to display a favorable change in property (when compared to the parental polypeptide, such as increased affinity or avidity to an antigen), it can be sequenced to identify the correspondingly favorable amino acid substitution contained therein.

In one aspect, upon mutagenizing each and every amino acid position in a parental polypeptide using saturation mutagenesis as disclosed herein, favorable amino acid changes may be identified at more than one amino acid position. One or more new progeny molecules can be generated that contain a combination of all or part of these favorable amino acid substitutions. For example, if 2 specific favorable amino acid changes are identified in each of 3 amino acid positions in a polypeptide, the permutations include 3 possibilities at each position (no change from the original amino acid, and each of two favorable changes) and 3 positions. Thus, there are 3×3×3 or 27 total possibilities, including 7 that were previously examined—6 single point mutations (i.e. 2 at each of three positions) and no change at any position.

In another aspect, site-saturation mutagenesis can be used together with another stochastic or non-stochastic means to vary sequence, e.g., synthetic ligation reassembly (see below), shuffling, chimerization, recombination and other mutagenizing processes and mutagenizing agents. This invention provides for the use of any mutagenizing process(es), including saturation mutagenesis, in an iterative manner.

Synthetic Ligation Reassembly (SLR)

Another non-stochastic gene modification, a “directed evolution process,” that can be can be used to modify a chimeric antigen binding polypeptide coding sequence has been termed “synthetic ligation reassembly,” or simply “SLR.” SLR is a method of ligating oligonucleotide fragments together non-stochastically. This method differs from stochastic oligonucleotide shuffling in that the nucleic acid building blocks are not shuffled, concatenated or chimerized randomly, but rather are assembled non-stochastically. See, e.g., U.S. patent application Ser. No. (USSN) 09/332,835 entitled “Synthetic Ligation Reassembly in Directed Evolution” and filed on Jun. 14, 1999 (“U.S. Ser. No. 09/332,835”). In one aspect, SLR comprises the following steps: (a) providing a template polynucleotide, wherein the template polynucleotide comprises sequence encoding a homologous gene; (b) providing a plurality of building block polynucleotides, wherein the building block polynucleotides are designed to cross-over reassemble with the template polynucleotide at a predetermined sequence, and a building block polynucleotide comprises a sequence that is a variant of the homologous gene and a sequence homologous to the template polynucleotide flanking the variant sequence; (c) combining a building block polynucleotide with a template polynucleotide such that the building block polynucleotide cross-over reassembles with the template polynucleotide to generate polynucleotides comprising homologous gene sequence variations.

SLR does not depend on the presence of high levels of homology between polynucleotides to be rearranged. Thus, this method can be used to non-stochastically generate libraries (or sets) of progeny molecules comprised of over 10¹⁰⁰ different chimeras. SLR can be used to generate libraries comprised of over 10¹⁰⁰⁰ different progeny chimeras. Thus, aspects of the present invention include non-stochastic methods of producing a set of finalized chimeric nucleic acid molecule shaving an overall assembly order that is chosen by design. This method includes the steps of generating by design a plurality of specific nucleic acid building blocks having serviceable mutually compatible ligatable ends, and assembling these nucleic acid building blocks, such that a designed overall assembly order is achieved.

The mutually compatible ligatable ends of the nucleic acid building blocks to be assembled are considered to be “serviceable” for this type of ordered assembly if they enable the building blocks to be coupled in predetermined orders. Thus the overall assembly order in which the nucleic acid building blocks can be coupled is specified by the design of the ligatable ends. If more than one assembly step is to be used, then the overall assembly order in which the nucleic acid building blocks can be coupled is also specified by the sequential order of the assembly step(s). In one aspect, the annealed building pieces are treated with an enzyme, such as a ligase (e.g. T4 DNA ligase), to achieve covalent bonding of the building pieces.

In one aspect, the design of the oligonucleotide building blocks is obtained by analyzing a set of progenitor nucleic acid sequence templates that serve as a basis for producing a progeny set of finalized chimeric polynucleotide molecules. These parental oligonucleotide templates thus serve as a source of sequence information that aids in the design of the nucleic acid building blocks that are to be mutagenized, e.g., chimerized or shuffled.

In one aspect of this method, the sequences of a plurality of parental nucleic acid templates are aligned in order to select one or more demarcation points. The demarcation points can be located at an area of homology, and are comprised of one or more nucleotides. These demarcation points are preferably shared by at least two of the progenitor templates. The demarcation points can thereby be used to delineate the boundaries of oligonucleotide building blocks to be generated in order to rearrange the parental polynucleotides. The demarcation points identified and selected in the progenitor molecules serve as potential chimerization points in the assembly of the final chimeric progeny molecules. A demarcation point can be an area of homology (comprised of at least one homologous nucleotide base) shared by at least two parental polynucleotide sequences. Alternatively, a demarcation point can be an area of homology that is shared by at least half of the parental polynucleotide sequences, or, it can be an area of homology that is shared by at least two thirds of the parental polynucleotide sequences. Even more preferably a serviceable demarcation points is an area of homology that is shared by at least three fourths of the parental polynucleotide sequences, or, it can be shared by at almost all of the parental polynucleotide sequences. In one aspect, a demarcation point is an area of homology that is shared by all of the parental polynucleotide sequences.

In one aspect, a ligation reassembly process is performed exhaustively in order to generate an exhaustive library of progeny chimeric polynucleotides. In other words, all possible ordered combinations of the nucleic acid building blocks are represented in the set of finalized chimeric nucleic acid molecules. At the same time, in another embodiment, the assembly order (i.e. the order of assembly of each building block in the 5′ to 3 sequence of each finalized chimeric nucleic acid) in each combination is by design (or non-stochastic) as described above. Because of the non-stochastic nature of this invention, the possibility of unwanted side products is greatly reduced.

In another aspect, the ligation reassembly method is performed systematically. For example, the method is performed in order to generate a systematically compartmentalized library of progeny molecules, with compartments that can be screened systematically, e.g. one by one. In other words this invention provides that, through the selective and judicious use of specific nucleic acid building blocks, coupled with the selective and judicious use of sequentially stepped assembly reactions, a design can be achieved where specific sets of progeny products are made in each of several reaction vessels. This allows a systematic examination and screening procedure to be performed. Thus, these methods allow a potentially very large number of progeny molecules to be examined systematically in smaller groups.

Because of its ability to perform chimerizations in a manner that is highly flexible yet exhaustive and systematic as well, particularly when there is a low level of homology among the progenitor molecules, these methods provide for the generation of a library (or set) comprised of a large number of progeny molecules. Because of the non-stochastic nature of the instant ligation reassembly invention, the progeny molecules generated preferably comprise a library of finalized chimeric nucleic acid molecules having an overall assembly order that is chosen by design.

The saturation mutagenesis and optimized directed evolution methods also can be used to generate these amounts of different progeny molecular species.

It is appreciated that the invention provides freedom of choice and control regarding the selection of demarcation points, the size and number of the nucleic acid building blocks, and the size and design of the couplings. It is appreciated, furthermore, that the requirement for intermolecular homology is highly relaxed for the operability of this invention. In fact, demarcation points can even be chosen in areas of little or no intermolecular homology. For example, because of codon wobble, i.e. the degeneracy of codons, nucleotide substitutions can be introduced into nucleic acid building blocks without altering the amino acid originally encoded in the corresponding progenitor template. Alternatively, a codon can be altered such that the coding for an originally amino acid is altered. This invention provides that such substitutions can be introduced into the nucleic acid building block in order to increase the incidence of intermolecularly homologous demarcation points and thus to allow an increased number of couplings to be achieved among the building blocks, which in turn allows a greater number of progeny chimeric molecules to be generated.

In another aspect, the synthetic nature of the step in which the building blocks are generated allows the design and introduction of nucleotides (e.g., one or more nucleotides, which may be, for example, codons or introns or regulatory sequences) that can later be optionally removed in an in vitro process (e.g. by mutagenesis) or in an in vivo process (e.g. by utilizing the gene splicing ability of a host organism). It is appreciated that in many instances the introduction of these nucleotides may also be desirable for many other reasons in addition to the potential benefit of creating a serviceable demarcation point.

Thus, according to another aspect, a nucleic acid building block can be used to introduce an intron. Thus, functional introns may be introduced into a man-made gene manufactured according to the methods described herein. The artificially introduced intron(s) can be functional in a host cells for gene splicing much in the way that naturally-occurring introns serve functionally in gene splicing.

Optimized Directed Evolution System

In practicing the methods of the invention, chimeric nucleic acids encoding an antigen binding polypeptide can also be modified by a method comprising an optimized directed evolution system. Optimized directed evolution is directed to the use of repeated cycles of reductive reassortment, recombination and selection that allow for the directed molecular evolution of nucleic acids through recombination. Optimized directed evolution allows generation of a large population of evolved chimeric sequences, wherein the generated population is significantly enriched for sequences that have a predetermined number of crossover events.

A crossover event is a point in a chimeric sequence where a shift in sequence occurs from one parental variant to another parental variant. Such a point is normally at the juncture of where oligonucleotides from two parents are ligated together to form a single sequence. This method allows calculation of the correct concentrations of oligonucleotide sequences so that the final chimeric population of sequences is enriched for the chosen number of crossover events. Thus provides more control over choosing chimeric variants having a predetermined number of crossover events.

In addition, this method provides a convenient means for exploring a tremendous amount of the possible protein variant space in comparison to other systems. Previously, if one generated, for example, 10¹³ chimeric molecules during a reaction, it would be extremely difficult to test such a high number of chimeric variants for a particular activity. Moreover, a significant portion of the progeny population would have a very high number of crossover events that resulted in proteins that were less likely to have increased levels of a particular activity. By using these methods, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

One method for creating a chimeric progeny polynucleotide sequence is to create oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. Additional information can also be found in U.S. Ser. No. 09/332,835. The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a ⅓ chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. By utilizing these methods, one can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events.

These methods are directed to the use of repeated cycles of reductive reassortment, recombination and selection that allow for the directed molecular evolution of a nucleic acid encoding an polypeptide through recombination. This system allows generation of a large population of evolved chimeric sequences, wherein the generated population is significantly enriched for sequences that have a predetermined number of crossover events. A crossover event is a point in a chimeric sequence where a shift in sequence occurs from one parental variant to another parental variant. Such a point is normally at the juncture of where oligonucleotides from two parents are ligated together to form a single sequence. The method allows calculation of the correct concentrations of oligonucleotide sequences so that the final chimeric population of sequences is enriched for the chosen number of crossover events. This provides more control over choosing chimeric variants having a predetermined number of crossover events.

In addition, these methods provide a convenient means for exploring a tremendous amount of the possible protein variant space in comparison to other systems. By using the methods described herein, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

In one aspect, the method creates a chimeric progeny polynucleotide sequence by creating oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. See also U.S. Ser. No. 09/332,835.

The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a ⅓ chance (assuming 3 parents) that a oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. One can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events.

Determining Crossover Events

Embodiments of the invention include a system and software that receive a desired crossover probability density function (PDF), the number of parent genes to be reassembled, and the number of fragments in the reassembly as inputs. The output of this program is a “fragment PDF” that can be used to determine a recipe for producing reassembled genes, and the estimated crossover PDF of those genes. The processing described herein is preferably performed in MATLAB® (The Mathworks, Natick, Mass.) a programming language and development environment for technical computing.

Iterative Processes

In practicing the methods of the invention, the process can be iteratively repeated. For example a nucleic acid (or, the nucleic acid) responsible for an altered antigen binding property is identified, re-isolated, again modified, re-tested for binding activity. The process can be iteratively repeated until a desired polypeptide is engineered. The invention is not limited to only a single round of screening. This iterative practice of determining which oligonucleotides are most related to the desired activity allows more efficient exploration all of the possible protein variants that might be provide a particular property or activity.

Mutagenized Oligonucleotides

While the optimized directed evolution method can use oligonucleotides that have a 100% fidelity to their parent polynucleotide sequence, this level of fidelity is not required. For example, if a set of three related parental polynucleotides are chosen to undergo ligation reassembly in order to create, e.g., an antibody with an altered binding affinity or specificity, a set of oligonucleotides having unique overlapping regions can be synthesized by conventional methods. However a set of mutagenized oligonucleotides could also be synthesized. These mutagenized oligonucleotides are preferably designed to encode silent, conservative, or non-conservative amino acids.

The choice to enter a silent mutation might be made to, for example, add a region of nucleotide homology two fragments, but not affect the final translated protein. A non-conservative or conservative substitution is made to determine how such a change alters the function of the resultant polypeptide. This can be done if, for example, it is determined that mutations in one particular oligonucleotide fragment were responsible for increasing the activity of a peptide. By synthesizing mutagenized oligonucleotides (e.g.: those having a different nucleotide sequence than their parent), one can explore, in a controlled manner, how resulting modifications to the peptide or protein sequence affect the activity of the peptide or polypeptide.

Another method for creating variants of a nucleic acid sequence using mutagenized fragments includes first aligning a plurality of nucleic acid sequences to determine demarcation sites within the variants that are conserved in a majority of said variants, but not conserved in all of said variants. A set of first sequence fragments of the conserved nucleic acid sequences are then generated, wherein the fragments bind to one another at the demarcation sites. A second set of fragments of the not conserved nucleic acid sequences are then generated by, for example, a nucleic acid synthesizer. However, the not conserved, sequences are generated to have mutations at their demarcation site so that the second fragments have the same nucleotide sequence at the demarcation sites as said first fragments. This allows the not conserved sequences to still hybridize during the ligation reaction to the other parental sequences. Once the fragments are generated, a desired number of crossover events can be selected for each of the variants. The quantity of each of the first and second fragments is then calculated so that a ligation/incubation reaction between the calculated quantities of the first and second fragments will result in progeny molecules having the desired number of crossover events.

Screening Methodologies and Devices

In practicing the methods of the invention and determining the properties of the chimeric antigen binding polypeptides of the invention any method or device can be used.

Capillary Arrays

Capillary arrays, such as the GIGAMATRIX™, Diversa Corporation, San Diego, Calif., can be used to screen for or monitor a variety of compositions, including the polypeptides and nucleic acids of the invention. Capillary arrays provide an efficient system for holding and screening samples. For example, a sample screening apparatus can include a plurality of capillaries formed into an array of adjacent capillaries, wherein each capillary comprises at least one wall defining a lumen for retaining a sample. The apparatus can further include interstitial material disposed between adjacent capillaries in the array, and one or more reference indicia formed within of the interstitial material. A capillary for screening a sample, wherein the capillary is adapted for being bound in an array of capillaries, can include a first wall defining a lumen for retaining the sample, and a second wall formed of a filtering material, for filtering excitation energy provided to the lumen to excite the sample.

A polypeptide or nucleic acid, e.g., an antibody, can be introduced into a first component into at least a portion of a capillary of a capillary array. Each capillary of the capillary array can comprise at least one wall defining a lumen for retaining the first component, and introducing an air bubble into the capillary behind the first component. A second component can be introduced into the capillary, wherein the second component is separated from the first component by the air bubble. A sample of interest can be introduced as a first liquid labeled with a detectable particle into a capillary of a capillary array, wherein each capillary of the capillary array comprises at least one wall defining a lumen for retaining the first liquid and the detectable particle, and wherein the at least one wall is coated with a binding material for binding the detectable particle to the at least one wall. The method can further include removing the first liquid from the capillary tube, wherein the bound detectable particle is maintained within the capillary, and introducing a second liquid into the capillary tube.

The capillary array can include a plurality of individual capillaries comprising at least one outer wall defining a lumen. The outer wall of the capillary can be one or more walls fused together. Similarly, the wall can define a lumen that is cylindrical, square, hexagonal or any other geometric shape so lone as the walls form a lumen for retention of a liquid or sample The capillaries of the capillary array can be held together in close proximity to form a planar structure. The capillaries can be bound together, by being fused (e.g., where the capillaries are made of glass), glued, bonded, or clamped side-by-side. The capillary array can be formed of any number of individual capillaries, for example, a range from 100 to 4,000,000 capillaries. A capillary array can form a microtiter plate having about 100,000 or more individual capillaries bound together.

Arrays, or “BioChips”

In one aspect of the invention, the chimeric polypeptides or nucleic acids of the invention can be analyzed by their immobilization onto an array, or “biochip.” Alternatively, antigen binding polypeptides can be screened by immobilizing antigens to an array. In practicing the methods of the invention, known arrays and methods of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. See also published U.S. patent applications Nos. 20010018642; 20010019827; 20010016322; 20010014449; 20010014448; 20010012537; 20010008765.

Antibodies and Immunoblots

In one aspect of the invention, animals are immunized before isolation of nucleic acids encoding antigen binding sequences. Methods of immunization, producing and isolating antibodies (polyclonal and monoclonal) are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

Sources of Cells and Culturing of Cells

Any vertebrate cell can be used as a source of nucleic acid encoding an antigen binding polypeptide. As noted above, immunoglobulin coding sequences can be isolated from cells of the immune system, e.g., B cells or plasma cells. Once a chimeric or modified antigen binding polypeptide coding sequence has been generated, it can be expressed in any cell, e.g., bacterial, Archaebacteria, mammalian, yeast, fungi, insect or plant cells. In one aspect, the cell can be from a tissue or fluid taken from an individual, e.g., a patient. The cell can be from, e.g., lymphatic or lymph node samples, serum, blood, chord blood, CSF or bone marrow aspirations, fecal samples, saliva, tears, tissue and surgical biopsies, needle or punch biopsies, and the like.

Any apparatus to grow or maintain cells can be used, e.g., a bioreactor or a fermentor, see, e.g., U.S. Pat. Nos. 6,242,248; 6,228,607; 6,218,182; 6,174,720; 6,168,949; 6,133,022; 6,133,021; 6,048,721; 5,660,977; 5,075,234.

Genetic Vaccines

The invention provides genetic vaccines comprising chimeric nucleic acids selected from the libraries of the invention. These genetic vaccines can be used in nucleic acid- or immunoglobulin-mediated immunomodulation. The invention provides various approaches for the evolution of genetic vaccines by stochastic (e.g. polynucleotide shuffling & interrupted synthesis) and non-stochastic polynucleotide reassembly.

A genetic vaccine is an exogenous polynucleotide that produces a medically useful phenotypic effect upon the mammalian cell(s) and organisms into which it is transferred. A genetic vaccine may be in the form of “naked” nucleic acid or as a vector. The vector or nucleic acid may or may not have an origin of replication. For example, it may be useful to include an origin of replication in a vector to allow for propagation of the vector in order to obtain sufficient quantities of the vector prior to administration to a patient. If the vector is designed to integrate into host chromosomal DNA or bind to host mRNA or DNA, or if replication in the host is otherwise undesirable, the origin of replication can be removed before administration, or an origin can be used that functions in the cells used for vector production but not in the target cells. However, in certain situations, including some of those discussed herein, it is desirable that the genetic vaccine vector be capable of replicating in appropriate host cells.

Vectors used in genetic vaccination can be viral or nonviral. Viral vectors are usually introduced into a patient as components of a virus. Exemplary vectors include, for example, adenovirus-based vectors (Cantwell (1996) Blood 88:4676-4683; Ohashi (1997) Proc. Nat'l. Acad. Sci USA 94:1287-1292), Epstein-Barr virus-based vectors (Mazda (1997) J. Immunol. Methods 204:143-15 1), adenovirus-associated virus vectors, Sindbis virus vectors (Strong (1997) Gene Ther. 4: 624-627), herpes simplex virus vectors (Kennedy (1997) Brain 120: 1245-1259) and retroviral vectors (Schubert (1997) Curr. Eye Res. 16:656-662). Nonviral vectors, typically dsDNA, can be transferred as naked DNA or associated with a transfer-enhancing vehicle, such as a receptor-recognition protein, liposome, lipoamine, or cationic lipid. This DNA can be transferred into a cell using a variety of techniques well known in the art. For example, naked DNA can be delivered by the use of liposomes which fuse with the cellular membrane or are endocytosed, i.e., by employing ligands attached to the liposome, or attached directly to the DNA, that bind to surface membrane protein receptors of the cell resulting in endocytosis. Alternatively, the cells may be permeabilized to enhance transport of the DNA into the cell, without injuring the host cells. One can use a DNA binding protein, e.g., HBGF-1, known to transport DNA into a cell. Furthermore, DNA can be delivered by bombardment of the skin by gold or other particles coated with DNA that are delivered by mechanical means, e.g., pressure. These procedures for delivering naked DNA to cells are useful in vivo. For example, by using liposomes, particularly where the liposome surface carries ligands specific for target cells, or are otherwise preferentially directed to a specific organ, one may provide for the introduction of the DNA into the target cells/organs in vivo.

### EXAMPLES

The following examples are offered to illustrate, but not to limit the claimed invention.

### Example 1

Building Genes Using an Exemplary Library and Method of the Invention

The following example describes building a nucleic acid, a gene, using an exemplary oligonucleotide library and method of the invention.

Building polynucleotides using the methods of the invention does not require handling of any template or parental DNA. Codon usage can be optimized towards any expression host. Restriction sites can be added/changed according to cloning needs.

This exemplary system of the invention uses a library of oligonucleotide building blocks to generate a DNA sequence. Oligonucleotide building blocks are designed for each sequence to be custom built. In one aspect, the library consists of all possible di-codon combinations at total of 4096 clones and 61 linker fragments. Oligonucleotide building blocks can be designed for each custom built sequence. Each oligonucleotide building block is cloned, sequence verified, PCR amplified (or prepped from a restriction digest) and pre-cut. See FIG. 1 for a summary of this exemplary iterative codon by codon gene building protocol.

Building Block Library Construction

A library of 4096 unique “building block” oligonucleotides is constructed in which each oligonucleotide (and corresponding clone into which the oligo is inserted) contains one specific di-codon sequence. The “building block” oligonucleotides are PCR amplified. “Starter” fragments to be linked to a solid support are precut at a 3′ codon. “Elongation fragments” are precut in a 5′ codon. The “starter” fragments (to be bound to solid support) and “elongation fragments” are cut with different Type-IIS restriction endonucleases; e.g., the starter” fragments are cut with EarI and the “elongation fragments” are precut with SapI, or, vice versa. In one example, “starter” fragments are first cut with BbsI for ligation to a “hook” and then cut with EarI after coupling to hook. “Elongation fragments” are amplified with primers SapF and T3 (a SapI site introduced during PCR) and cut with SapI. In one exemplary protocol, PCR amplification of the building block oligonucleotides adds a SapI site and deletes the EarI site. Each “building block” oligonucleotides is cloned and each dicodon sequence verified.

In this exemplary method, the cloning vector into which each oligonucleotide building block is inserted is a modification of pBluescriptII Ks minus™ (Stratagene, San Diego, Calif.). The following changes were made:

Removal of Vector-specific SapI and EarI Sites:

As in some aspects SapI and EarI are used to generate overhangs in the building block oligonucleotides, it is necessary to remove SapI and EarI recognition sites in the vectors. In this example, pBluescriptII Ks minus™ contains three EarI sites (at positions 518, 1038 and 2842), one of them overlapping a single SapI site (at position 1038). These sites can be removed by, e.g., using Stratagene's QUICKCHANGE SITE DIRECTED MUTAGENESIS™ kit. Successful changes can be verified by restriction cuts using SapI and EarI and/or sequencing. In this example, the modified vector was designated pΔSE.

Insertion of a Single BbsI Site:

The “starter fragments” need to be ligated to the “hook” immobilized on the solid support, in this example, the hook is immobilized to magnetic beads. A non-palindromic overhang (e.g., 5′-GGGG-3′) can be used in order to avoid self-ligation of the fragments. The sequence is available by insertion of this double stranded fragment into the pASE vector (see above) and with SacI/NotI. In to the linearized vector insert:

(SEQ ID NO:15)

SacI ↓ NotI
5′-AGCTCGAAGACTTGGGGTTGTCTTCACCGCGGTGGC

(SEQ ID NO:16)

```
        3′-GCTTCTGAACCCCAGAATGGCGCCACCGCCGG-5′
              BbsI ↑
```

This introduces BbsI site to create GGGG overhangs for high ligation efficiency (connection to hook fragment on solid support). Annealing of equal molar amount of PAGE purified oligonucleotides (e.g., from Integrated DNA Technologies, Coralville, Iowa) will create the double stranded (ds) fragment as shown above. Successful integration can be verified by restriction cut with BbsI and sequencing. The BbsI site is designed to generate a 5′-GGGG overhang. This modified vector is designated pBbs4G. This vector (pBbs4G) can be used for making the library.

Insertion of Sma/PstI Spacer

In this example, inserts of the oligonucleotide library have blunt ends on one side and PstI compatible 3′-overhangs on the other enabling directed cloning without further manipulation into a SmaI/PstI cut vector. These sites are located directly next to each other in the pBluescriptII Ks minus™ (Stratagene, San Diego, Calif.) vector. After the first enzyme cuts, the recognition sequence of the other one is very close to the end of the DNA. PstI and SmaI do not cut efficiently close to DNA ends. This problem can be solved by inserting this dsDNA into the vector pBbs4G cut with SmaI and HindIII, dephosphorylated and gel purified:

Cut pBbs4G with SmaI/HindIII, insert:
SmaI (half) PstI EcoRI HindIII

(SEQ ID NO:17)

5′-GGGCATCATCATCATCATCTGCAGGAATTCGATATGA

(SEQ ID NO:18)

3′-CCCGTAGTAGTAGTAGTAGACGTCCTTAAGCTATACTTCGA

Separate SmaI and PstI to make double cuts more efficient. The fragment can be generated by annealing complementary, 5′-phosphorylated oligonucleotides, as noted above. Successful integration can be checked by sequencing. The modified vector is designated pGB1. KpnI or SacI can be used instead of PstI without vector modification, but this may result in much shorter fragments (see below) which are more difficult to prepare (the efficiency of standard methods drops below about 70 base pairs).

Design of the Building Blocks

In this exemplary procedure, to start gene synthesis with any codon simultaneously at several starting points a total of 61 “starter” and 4096 “elongation” fragments are used. All fragments can be cloned into pGBI (see above). The vector can be cut with SmaI and PstI, dephosphorylated and gel purified.

“Starter fragments ”

The 61 “starter” clones can be created by annealing two partially complementary oligonucleotides, as illustrated below. Filling in the 5′ overhangs with Klenow DNA polymerase and cloning the mixture into pGB1 as described above. SapI can be used to generate the overhang for ligation of the first elongation fragment. BsmFI can be used to release partial genes from the solid support and ligate those to generate full length genes. The vector is cut with SmaI/PstI.

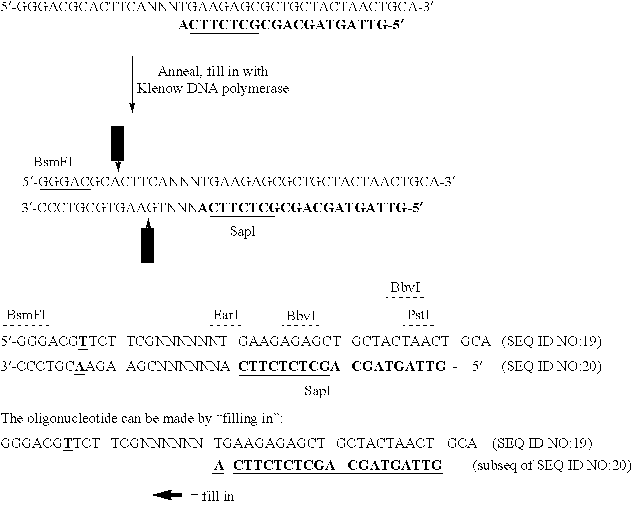

In one aspect, 96 colonies are picked and sequenced. Missing codons can be created using a sequence-specific primer instead of a degenerate primer. The cloning procedure is the same as outlined above.

“Elongation Fragments”

The “Elongation Fragments” containing all possible 4096 dicodon combinations (all possible two-codon combinations) can be generated according to the procedure as described above. The oligos used are as follows:

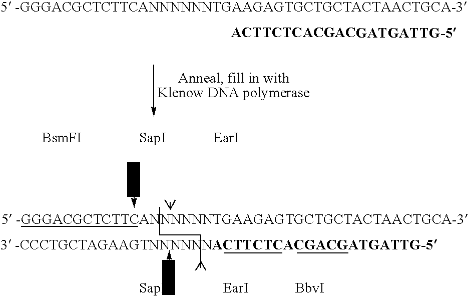

The clones have this design:

| | |
|---|---|
| SacI BbsI NotI SpeI | |
| T7 promoter ˜˜˜˜˜˜ ˜˜˜˜˜˜ ˜˜˜˜˜˜˜˜ ˜˜˜˜˜˜ | |
| CGCGCGTAATACGACTCACTATAGGGCGAATTGGAGCTCGGGGTTGTCTTCACCGCGGTGGCGGCCGCTCTAGAACTAGT | (SEQ ID NO:21) |
| GCGCGCATTATGCTGAGTGATATCCCGCTTAACCTCGAGCCCCAACAGAAGTGGCGCCACCGCCGGCGAGATCTTGATCA | (SEQ ID NO:22) |
| Primer E_F | |
| BamHI BsmFI EarI BbvI PstI EcoRI HindIII ClaI | |
| ˜˜˜˜˜˜ ˜˜˜˜˜ ˜˜˜˜˜˜ ˜˜˜˜˜ ˜˜˜˜˜˜˜˜˜˜˜˜ ˜˜˜˜˜˜˜˜˜˜˜˜ | |
| GGATCCCCCTGGGACGTTCTTCGNNNNNNTGAAGAGAGCTGCTACTAACTGCAGGAATTCGATATGAAGCTTATCGATAC | |
| CCTAGGGGGACCCTGCAAGAAGCNNNNNNACTTCTCTCGACGATGATTGACGTCCTTAAGCTATACTTCGAATAGCTATG | |
| SalI XhoI KpnI | |
| ˜˜˜˜˜˜˜˜˜˜˜˜ ˜˜˜˜˜˜ T3 promoter | |
| CGTCGACCTCGAGGGGGGGCCCGGTACCCAGCTTTTGTTCCCTTTAGTGAGGGTTAATTGCGCGCTTGGCGTAATCATGG (a) | |
| GCAGCTGGAGCTCCCCCCCGGGCCATGGGTCGAAAACAAGGGAAATCACTCCCAATTAACGCGCGAACCGCATTAGTACC (b) | |

SapI is used to generate 5′ overhangs prior to the ligation. EarI is used to create 5′ overhangs in the next codon for addition of the next fragments. BsmFI and BbvI restriction sites are positioned to enable cutting within the first two and last two codons of a synthesized DNA fragment. BsmFI is used to release partial genes from the solid support. BbvI is used to generate compatible overhangs at the 3′ end of partial genes attached to the solid support.

The library comprises 4096 clones. Two of the clones (coding for the sequence CTCTTC and GAAGAG) cannot be used for the assembly process because they encode the EarI recognition sequence. This is not a problem because the target sequences can be modified accordingly. In order to capture and conserve the entire variability, 10,000 single colonies are picked into 96-well plates. An automated colony picker can be used for this purpose. In one aspect, it is sufficient to have 96 unique clones. In one aspect, enough clones are sequenced to be able to synthesize an artificial gene of one kbp in length.

In one aspect, only four different class IIS restriction enzymes (SapI, EarI, BsmFI, BbvI) are used to generate compatible overhangs for the ligation of the individual building blocks. SapI and EarI generate 3-base 5′ overhangs, BsmFI and BbvI 4-base 5′ overhangs. The design of the starter/elongation clones is shown in Table 2:

TABLE 2
Design of the building blocks.

```
Starter clones
          T7 primer SacI BbsI NotI XbaI
TAATACGACTCACTATAGGGCGAATTGGAGCTCGAAGACTTGGGGTCTTACCGCGGTGGCGGCCGCTCTA
ATTATGCTGAGTGATATCCCGCTTAACCTCGAGCTTCTGAACCCCAGAATGGCGCCACCGCCGGCGAGAT
                                  BsmFI SapI BbvI PstI EcoRI
GAACTAGTGGATCCCCCGGGACGCACTTCANNNTGAAGAGCGCTGCTACTAACTGCAGGAATTCGATATG
CTTGATCACCTAGGGGGCCCTGCGTGAAGTNNNACTTCTCGCGACGATGATTGACGTCCTTAAGCTATAC
            ClaI SalI XhoI KpnI
AAGCTTATCGATACCGTCGACCTCCAGGGGGGGCCCGGTACCCAGCTTTTGTTCCCTTTAGTGAGGCTTA
TTCGAATAGCTATGGCAGCTGGAGCTCCCCCCCGGGCCATGGGTCGAAAACAAGGGAAATCACTCCCAAT
                                                                                                                          T3 primer
Elongation clones
T7 primer SacI BbsI NotI XbaI
TAATACGACTCACTATAGGGCGAATTGGAGCTCGAAGACTTGGGGTCTTACCGCGGTGGCGGCCGCTCTA
ATTATGCTGAGTGATATCCCGCTTAACCTCGAGCTTCTGAACCCCAGAATGGCGCCACCGCCGGCGAGAT
```

Starter fragments. The inserts can be recovered as restriction fragments (BbsI/KpnI; 140 bp) or by amplification with T7/T3 primers (210 bp) and a restriction cut with BbsI (170 bp).

Elongation fragments. The inserts can be recovered as restriction fragments (SapI/KpnI; 88 bp) or by amplification with S1/T3 primers (127 bp) and a restriction cut with SapI (110 bp).

Preparation of Building Blocks:

Starter and elongation fragments can be generated by PCR, purified by using, e.g., the Qiagen PCR purification kit, digested by SapI, and purified again by using a Qiagen PCR purification kit. These processes can be carried out in a 96-well format on, e.g., a Beckman BIOMEK 2000™. The standard operation protocols are used. The purified building blocks can be stored at a standardized DNA concentration (e.g. 100 pmol/μl) in 96-well deep blocks (up to 2 ml).

It is not anticipated that PCR-introduced nucleotide substitution will cause a significant number of mutations in the synthesized gene. A THERMALACE™ DNA polymerase (Invitrogen) can be used; it is a high fidelity/high efficiency enzyme. The error rate is 1/(6×10⁵). This means one out of 1500 copies of a 200 bp PCR product (600,000b:400 b) has one error on average. Only 6 bp (12 bases) of each fragment are used for the synthesis. The probability that one of these bases is wrong is only 3% for a 200 bp product (12:400). Therefore only one out of 50,000 copies has an error introduced in the di-codon region (=0.002%; compared to synthetic oligos: 2-5%). Mutations outside of the di-codon region do not carry through to the synthesized sequence.

Mutated codons are further discriminated during ligation. Several hundred clones from synthetic genes and gene reassembly projects have been sequenced and no introduced base error or missing/wrong bases have been seen in the overhang region.

Plasmid preparation is an alternative to PCR amplification. Building blocks can be prepared from restriction digestion of the plasmid DNA. The fragments can be purified from its vector backbone by a size-fractionation column. This method is an alternative if nucleotide substitution causes a high mutation rate.

The Elongation Protocol

In one aspect, the elongation cycle involves 3 steps: (1) covalent linkage of the new fragment by DNA ligase, (2) fill-in the unligated overhangs by Klenow DNA polymerase, and (3) restriction digestion by EarI to generate the next overhang. Each step can be optimized separately, and then synthesize several short DNA sequences (30-60 bp) to test and optimize the entire synthesis cycle. The synthesized fragments can be cloned and sequenced to verify the efficiency and the fidelity of the elongation reactions.

In one aspect, reassembly of DNA molecules from synthetic oligonucleotides using the solid-phase support is applied to the reassembly of gene families. In this protocol, full-length reassembled genes were obtained by step-wise ligation of annealed oligonucleotides of 30-50 bases.

Two different sets of building blocks need to be prepared from the library's “archived” clones:

starter fragments

can be linked to solid support

amplification with primers E_F and T3

cut with BbsI for ligation to hook

cut with EarI after coupling

elongation fragments

amplification with primers SapF and T3

SapI site introduced during PCR

Cut with SapI

Used to elongate starter fragments by one codon/elongation cycle

Hook for Linking Starter Fragments to Solid Support: Immobilization of the Hook Fragment

Paramagnetic beads coated with Streptavidin can be purchased from Dynal A. S. (Oslo, Norway). The 5′- biotinylated forward oligo (5′-bio-GAACGATAATAAGCTTGATGACGAAGACAT-3′) (SEQ ID NO:23) and the reverse oligo (5′-CCCCATGTCTTCGTCATCAAGCTTATTATCGTTC-3′) (SEQ ID NO:24) can be purchased, e.g., from Integrated DNA Technologies Inc. (Coralville, Iowa). The two oligonucleotides can be annealed to generate the hook fragments. The hook fragments can be immobilized to the beads according to manufacturer's instructions (e.g., the Dynal protocol).

| | |
|---|---|
| T7 promoter | |
| (NNN)_{X}CGCGCGTAATACGACTCACTATAGGGCGAATTGGAGCTC | (SEQ ID NO:25) |
| (NNN)_{X}GCGCGCATTATGCTGAGTGATATCCCGCTTAACCTCGAGCCCC | (SEQ ID NO:26) |

Preparation of “Hook”:

length/sequence variable

may contain promoter (e.g. T7) for in vitro transcription/translation

compatible overhang for ligation of starter fragments

Alternative Method:

Instead of using PCR fragments derived from sequence verified clones, building blocks are synthesized from short (about 20 to 25 base pairs (bp)) double stranded (ds)DNA fragments derived from oligos. Only the 3 bases at the 3′ end of the bottom strand (see figure) are critical for building a correct sequence.

Principle:

>solid support<—hook—starter fragment—codon specific overhang

Hook for linking starter fragments to solid support:

| | |
|---|---|
| T7 promoter | |
| (NNN)_{X}CGCGCGTAATACGACTCACTATAGGGCGAATTGGAGCTC | (SEQ ID NO:27) |
| (NNN)_{X}GCGCGCATTATGCTGAGTGATATCCCGCTTAACCTCGAGCCCC | (SEQ ID NO:28) |

Starter Fragment:

| | |
|---|---|
| BsmFI | |
| GGGGATCCTGGGACGTTCTTCG | (SEQ ID NO:29) |
| TAGGACCCTGCAAGAAGCNNN | (SEQ ID NO:30) |

Building Blocks:

(SEQ ID NO:31)
NNNnnnTGAAGAGAGCTGCTACTAACTGCAGGAATTCGATATGAAGCTT
(SEQ ID NO:32)

nnnACTTCTCTCGACGATGATTGACGTCCTTAAGCTATACTTCGAA

In summary, as illustrated in FIG. 1, the “elongation cycle” of this exemplary gene building method of the invention comprises: “loading” starter oligo onto substrate; ligation (with any ligase, e.g., T4 ligase or E. coli ligase); wash; fill-in ends; wash; cut with restriction endonuclease; wash; repeat (reiterate cycle). Any type of protocol or alternative protocols can be used. Optimization of conditions can be done by routine screening of a range of parameters, e.g., temperature, time, buffers, number of elongation cycles, which ligase to use, choice of solid substrate, if any, and the like.

Ligation

Enzymes

In one aspect, the T4 DNA ligase is used; it is the most commonly used enzyme in DNA ligation reactions. It has a high specific activity and joins 5′ or 3′ protruding compatible overhangs very efficiently. It also ligates blunt-ended fragments but at a lower efficiency. This creates a possible problem, because the building blocks (if generated by PCR) are blunt-ended on one side and could ligate to other blunt-ended fragments resulting from the fill-in reaction. Dimerization of building blocks will not be a problem because non-phosphorylated primers are used for PCR. In one aspect, to avoid these side reactions E. coli DNA ligase can be used as an alternative to T4 DNA ligase. E. coli DNA ligase is NAD⁺-dependent and ligates only cohesive ends of DNA fragments. It has a 1 to 2 order of magnitude higher fidelity but lower specific activity than T4 DNA ligase. The E. coli DNA ligase is commercially available. Using routine screening protocols, both enzymes can be evaluated to determine the most efficient procedure under desired conditions.

Optimization

Using routine screening protocols, the ligation efficiency under different conditions can be optimized for, e.g., desired results, materials and/or conditions. Three parameters can be optimized, DNA concentration, enzyme units, and reaction time. A fluorescence (e.g. 6-Fam) labeled T3 primer (see Table 2 above) can be used with an unlabeled S1 primer in PCR reactions, using known di-codon clones as templates, to generate labeled elongation fragments. Several labeled fragments can be generated to cover different GC content in the overhangs. These fragments can be used to monitor the ligation efficiency during protocol development. In each reaction, one of the labeled fragments can be used as the last one to be added to the elongation chain (2 to 3 codons for the purpose of protocol development). Upon completion of the reaction, the fragments can be released from the solid-support and incorporated label can be analyzed, e.g., on an ABI PRISM 310 GENETIC ANALYZER™. A method as described by, e.g., Liu (1997) Appl. Environ. Microbiol 63:4516-4522, can be used.

Fill-in Reaction

Enzymes

In the ligation step, a molar excess of the next building block can be used to saturate the fragments attached to the beads and to drive the ligation to completion. The methods of the invention can be a multi-step process; therefore, even trace amounts of un-ligated fragments could reduce the accuracy and quality of the final product. To prevent un-ligated fragments from elongation in later cycles (same codon), a Klenow DNA polymerase can be used after each ligation step to fill in un-ligated overhangs. Klenow DNA polymerase has the advantage of being active in almost all commonly used restriction buffers avoiding additional buffer exchange. In one aspect, the enzyme is inactivated, e.g., heat-inactivated, before the next ligation step.

Optimization Fill-in Conditions

Using routine screening protocols, fill-in reaction conditions can be optimized for, e.g., desired results, materials and/or conditions. In one aspect, to optimize reaction conditions (fill in of all ends), a DNA fragment (30-40 bp) is used with a 3-base 5′ overhang as a substrate for the reaction. Two complementary oligos can be designed. The forward oligo can contain a 5′ fluorescence (e.g. 6-Fam) label. The reverse primer can be 3-bases longer at the 5′ than the forward oligo. Annealing of these two oligos will generate a fluorescence labeled DNA fragment with a 3-base 5′ overhang. The annealed fragment can be used as the substrate for the optimization of the fill-in reaction. Upon the completion of the reaction, the sample will be analyzed on, e.g., an ABI PRISM 310 GENETIC ANALYZER™ as described above.

The percentage of the unfilled fragment (same length as the forward oligo), partially filled fragments (one or two bases longer than the forward oligo), and completely filled fragment (same length as the reverse oligo) can be determined to assess the efficiency of the fill-in reaction. The fill-in reaction has to be optimized regarding (1) enzyme concentration, (2) buffer composition, (3) incubation time, and (4) inactivation temperature/time.

Restriction Digest Optimization

In one aspect, EarI is used after the fill-in reaction to generate a new overhang. Optimization of this step can include enzyme concentration and incubation time. A strategy similar to the one used for the optimization of the ligation reaction will be used for this reaction. A labeled building block can be linked to the hook fragment by ligation and cut with EarI. Release of labeled fragment can be analyzed on, e.g., an ABI PRISM 310 GENETIC ANALYZER™ as described above.

Software Development and Automation

Manipulation of a Target Sequence

To manipulate a sequence that is synthesized by the methods of the invention, silent mutations can be performed for host optimization and/or for the elimination of restriction sites for EarI, SapI, BsmFI and/or BbvI I in the sequence (e.g., newly synthesized gene). In one aspect, sequence manipulation is determined by software analyses in preparation for synthesis by the methods of the invention. In one aspect, silent mutations for both codon optimization and restriction site manipulation are performed.

Automation for Building Block Preparation

In one aspect, preparation of building blocks is performed on a Beckman BIOMEK 2000™ using off-the-shelf software and preparation kits. These operations are currently standard procedures; no further development are required to perform this step of the protocol.

Software to Generate a Sequence from Available Building Blocks

If not all building blocks are available, it may be necessary for a sequence to be built from the available material. A software application can be written that takes the sequencing results of the available building blocks into account and creates a feasible sequence. The software can loop through all wells in the experiment and create a database of all other wells that have the complimenting sequence. To create the sequence the software can pick a building block to start with and chooses randomly from all of the building blocks that can be added to that one. The system can repeat this process for as many building blocks as are required for the desired length.

Automation to Execute the Elongation Protocol

To execute the elongation protocol, an automation system can be developed that will read a file containing the gene sequence into memory and command a Beckman BIOMEK 2000™ robot to perform the steps in the protocol. To choose building blocks, the software can read the first and second codon in the sequence being synthesized. That sequence uniquely identifies a building block that can then be pipetted from the appropriate building block material plate. After loading the building block material, the robot can automatically perform the remainder of the elongation cycle. The next building block can be determined from the second and third codons in the sequence. This process can be repeated until the gene is complete.

Synthesis of an Artificial Gene

In one aspect a gene for an artificial protein sequence with a length of about 300 residues is generated based on the available di-codon clones. The gene can be synthesized according to the optimized elongation protocol, as discussed above. To maximize efficiency, small, equally sized fragments can be synthesized in parallel (round I). These partial genes can be used as building blocks in round II to generate the full-length gene. The number of codons per fragment in round I can be determined by the maximum number of cycles, which can be carried out from one starting point (see below).

Up to 22 fragments have been joined in using the exemplary protocol of the invention. For a gene of 300 codons, 14 fragments can be synthesized in parallel in the round I of synthesis. In the second round of the synthesis, 13 fragments can be ligated to the first fragment sequentially. The length of the incoming fragment may have little or no effect on the ligation efficiency. Thus, the efficiency of the second round synthesis of the 14 fragments can be similar to the first round synthesis.

The same artificial gene can be synthesized using oligos and a standard solid-phase protocol. Oligos can be ordered from a commercial source, e.g., Integrated DNA Technologies, and ligated to synthesize the full-length gene. This product can be used as a control to evaluate the efficiency and accuracy of additional products of the methods of the invention, as compared to a traditional method. At least 20 clones from each experiment can be sequenced and compared.

### Example 2

Antibody Reassembly

The following example describes implementation of the antibody reassembly methods of the invention to generate chimeric antigen binding polypeptides.

Reassembly Strategy:

A cloning vector was designed as schematically illustrated in FIG. 1. Any ribosome binding site (RBS) sequence or green fluorescent protein coding sequence (GFP) can be used, may of which are well known in the art.

Reassembly Strategy for Lambda Light Chains:

To reassemble lambda light chains, three domains were provided:

V_{L}: 38 sequences in 10 families; about 300 base pairs (bp) in length (˜300 bp)

J_{L}: 4 sequences; about 35 base pairs (bp) in length (˜35 bp)

C_{L}: 1 sequences; about 320 base pairs (bp) in length (˜320 bp)

→38×4×1=154 different combinations

V_{L} sequences were PCR amplified with gene specific primers:

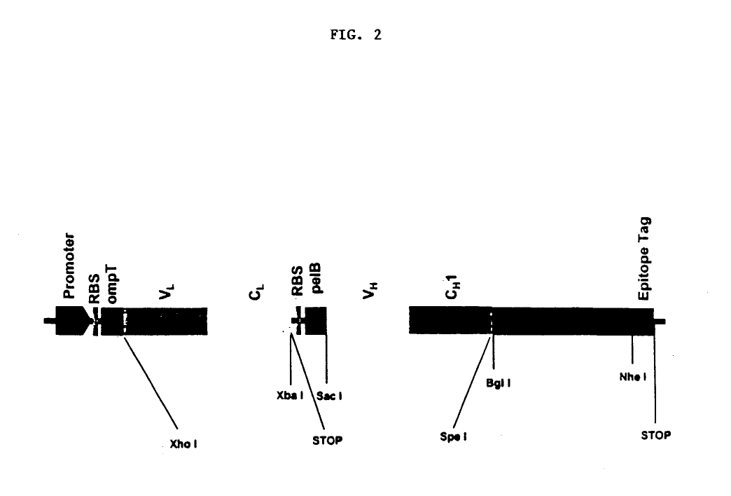

=> 5′ oligos are designed with a XhoI site; 3′ primers are designed with extension/SapI site (see scheme in FIG. 2);

=> J_{L} sequences are generated from oligos (see FIG. 2 and SEQ ID NO:1, SEQ ID NO:2, SEQ ID NO:3, SEQ ID NO:4);

=> C_{L} sequence is PCR amplified with an oligo including a BsrDI site at the 5′ end and a XbaI site at the 3′ end.

Because only 1 V_{L} gene has an internal SapI site:

→37×4×1=148 combinations

FIG. 2 schematically illustrates an exemplary scheme to reassemble lambda light chains according the methods of the invention. J region oligos (in the center shaded box) are SEQ ID NO:1, SEQ ID NO:2, SEQ ID NO:3, SEQ ID NO:4.

Primers for PCR amplification of V_{λ} and C_{λ} are:

Reverse primer V_{λ} add-on:

CATCATGCTCTTCACACMNM (SEQ ID NO:5) plus gene specific sequence (M=C or A)

Forward primer C_{λ}5′ add-on:

CTACTAGGTCTCATCCTG (SEQ ID NO:6) plus gene specific sequence; (last codon in J region changed from CTA to CTG because of codon usage in E. coli).

Reassembly Strategy for Kappa Light Chains:

To reassemble lambda light chains, three domains were provided:

V_{K}: 49 sequences in 7 families; about 300 base pairs (bp) in length (˜300 bp)

J_{K}: 5 sequences; about 35 base pairs (bp) in length (˜35 bp)

C_{K}: 1 sequences; about 320 base pairs (bp) in length (˜320 bp)

→49×5×1=254 combinations

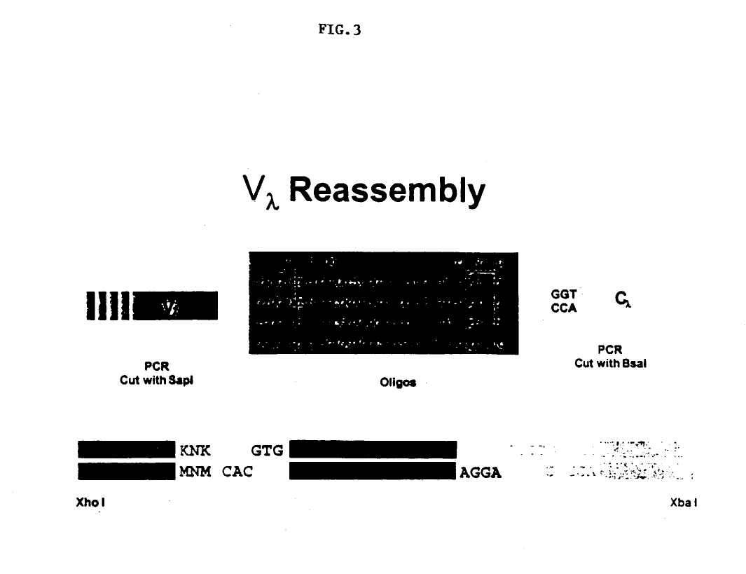

FIG. 3 schematically illustrates an exemplary scheme to reassemble kappa light chains according the methods of the invention. J region oligos (in the center shaded box) are SEQ ID NO:7, SEQ ID NO:8, SEQ ID NO:9, SEQ ID NO:10; SEQ ID NO:11.

V_{K} sequences were PCR amplified with gene specific primers:

=> 5′ oligos are designed with XhoI sites and 3′ primers are designed with extension BsrDI sites (see scheme in FIG. 3);

=> J_{K} sequences are generated from oligos (see FIG. 3 and SEQ ID NO:7, SEQ ID NO:8, SEQ ID NO:9, SEQ ID NO:10; SEQ ID NO:11);

=> C_{K} sequences are PCR amplified using oligos including a BsaI site at the 5′ end and a XbaI site at the 3′ end.

Primers for PCR amplification of V_{κ} and C_{κ} are:

Reverse primer V_{κ} add-on:

CATCATGCAATG (SEQ ID NO:12) plus gene specific part (the first base of the last codon is skipped)

Forward primer C_{κ}5′ add-on:

CTACTAGGTCTCAAA (SEQ ID NO:13) plus gene specific sequence.

Reassembly of Heavy Chains:

Immunoglobulin heavy chains were reassembled with four domains:

V_{H}: 57 sequences in 7 families; ˜300 bp

D_{H}: 116 sequences (both orientations, different reading frames included); ˜20 bp

J_{H}: 12 sequences; ˜60 bp

C_{H}: 1 sequence; ˜300 bp

→57×116×12×1=79344 combinations

Reassembly Strategy:

PCR amplify V_{H} genes with gene specific primer

Primers include SacI site at 5′ end

Primers include Sap I site at 3′ end to generate 3bp overhangs in last codon; last codon is AGA for most genes (45 out of 57)

V_{D} and V_{J} genes are synthesized from oligos (see scheme below); first library targets only AGA junctions and TAC junctions (7 of 12 J's)

PCR amplify CH gene, including a BsaI or BsmBI site at the 5′ end and a SpeI site at the 3′ end

→45×116×7×1=36540

Primers for PCR amplification of V_{H} and C_{H} are:

Reverse primer V_{H} add-on:

CATCATGCTCTTCA (SEQ ID NO:14) plus gene-specific part Forward primer C_{H}5′ add-on:

CTACTAGGTCTC (SEQ ID NO:15) plus gene specific part

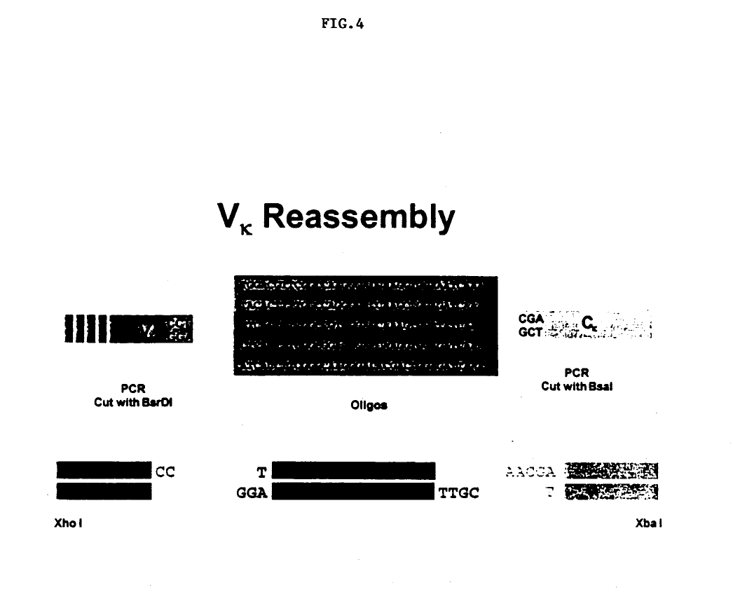

FIG. 4 schematically illustrates an exemplary scheme to reassemble antibody heavy chains according the methods of the invention.

### Example 3

Approaches to Step-wise Nucleic Acid Reassembly: Tandem Reassembly

The following example described an exemplary procedure of the invention. For example, step-wise nucleic acid reassembly (i.e., “Tandem Reassembly”) can be used in conjunction with the nucleic acid synthesis methods of the invention. In one aspect, step-wise nucleic acid reassembly is used to assemble nucleic acids made by iterative assembly of oligonucleotide building blocks using the compositions and methods of the invention. In one aspect, step-wise nucleic acid reassembly is used to further modify the chimeric antibodies of the invention. In one aspect, the products of step-wise nucleic acid reassembly are isolated and/or purified using the invention's compositions and methods for purifying double-stranded polynucleotides lacking base pair mismatches, insertion/deletion loops and/or nucleotide gaps.

This example is provided to illustrate an exemplary step-wise application of a reassembly nucleic acid. This step-wise approach can allow the construction of products to be expedited by allowing the construction of partial reassembly products (or reassembly sub-products or intermediate reassembly products) to occur simultaneously or in parallel, and for these partial reassembly products to then be assembled into final products. The following example illustrates this step-wise reassembly approach using 3 partial products, but in different aspects of this invention, different numbers of partial products can be used (e.g. corresponding to every integer value from 2 to one billion). In this approach, pools of nucleic acid fragments (or nucleic acid building blocks) containing sequences from each gene (or other sequence, e.g. gene pathway or regulatory motif), to be reassembled are stepwise ligated but not to full length.

In this example, the assembly process was started from three positions within the sequences: the 5′-end, an internal position (Internal) and the 3′-end. Overhangs at the junction points are designed to accommodate a biotinylated hook containing appropriate restriction sites (e.g. the solid phase protocol according to Dynal A. S., Oslo, Norway, see Biomagnetic Techniques in Molecular Biology—Technical Handbook, 3rd edition, section 5.1 entitled: “Solid-phase gene assembly”, page 135-137).

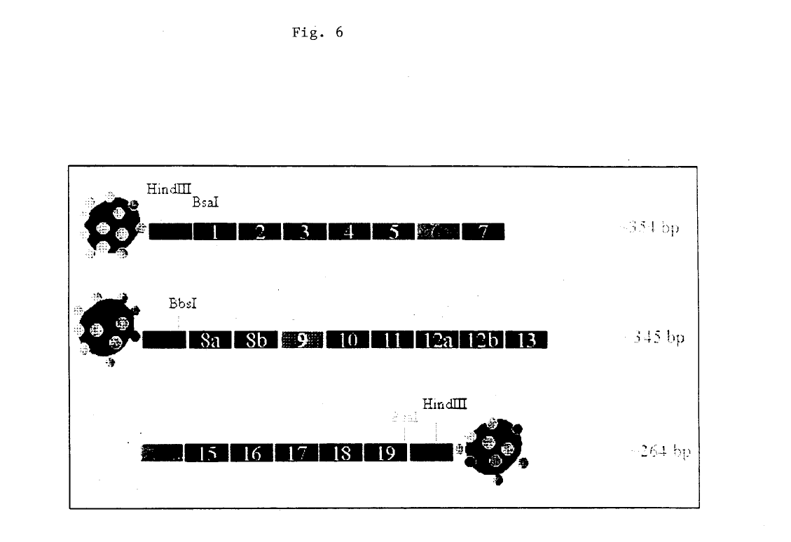

The example illustrated in FIG. 6 is for the reassembly of three esterase genes (a “three points ligation approach” for the reassembly of three esterase genes). After alignment of the three parental sequences, overhangs were designed and corresponding oligos were synthesized. Prior to the reassembly, analog sequences were pooled into one sample and 19 pools of nucleic acid building blocks were created (the 19 nucleic acid building blocks were named F1 to F19). Reassembly was carried out with the pools following standard procedures. Three sub-products were made: F1-7, F8a-13 and F14-19. Assembly processes were performed either in the 5′-3′ direction of the genes or, e.g. for the F14-19 intermediate product, in the 3′ to 5′ direction.

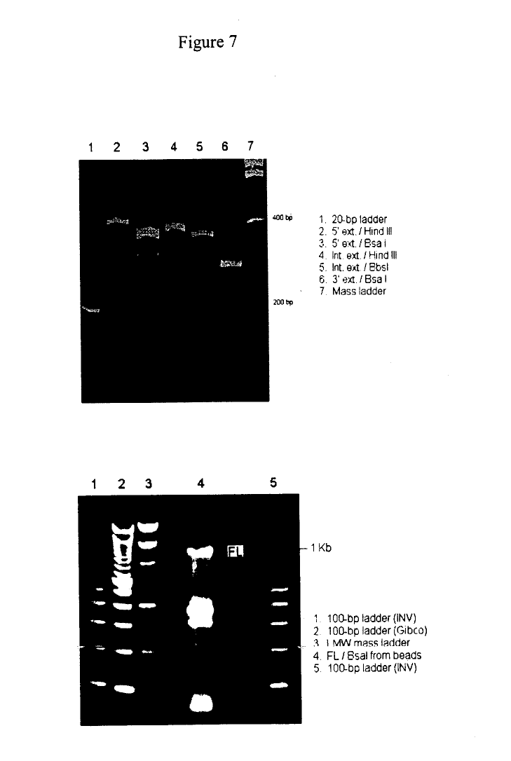

Once the three sub-products were made using solid phase bead supports, the F8a-13 and F14-19 sub-products were released from the beads using shift restriction enzymes (see FIG. 7A), e.g. Bsa I or Bsb I (other can be used as well). FIG. 7A illustrates the elution of reassembled DNA from the solid support using alternative restriction sites engineered in the biotinylated hook. Eluted F1-7 (lanes 2-3), eluted F8a-13 (lanes 4-5), and eluted F14-F19 (lane 6). DNA ladders (lanes 1 and 7).

The released F8a-13 was then assembled onto the bead-attached F1-7 sub-product, followed by the assembly of the F14-19 sub-product. Sub-products F8a-13 and F14-19 can be added in molar excess to facilitate the generation of full-length products. FIG. 7B shows the elution of final reassembled products. FIG. 7B illustrates the elution of final reassembled products from the solid support (lane 4). DNA ladders (lanes 1, 2, 3, and 5). Thus, the intended full-length product was gel purified for cloning and library generation.

### Example 4

An exemplary oligonucleotide purifying protocol: “MutS treatment”

This example describes an exemplary oligonucleotide purifying method of the invention, “MutS treatment.”

Reassembly of the 1658 OT5 Gene

This example illustrates that the treatment of reassembly fragments (or nucleic acid building blocks) with a MutS protein-based filtering (or purification) step substantially increased the yield of intact open reading frames that resulted from the nucleic acid reassembly process of the invention. To demonstrate this, the gene of a fluorescent protein was synthesized from nucleic acid building blocks with or without prior MutS treatment.

From the 732 base pair (bp) gene sequence for the fluorescent protein 1658 OT5 suitable nucleic acid building blocks were designed and the corresponding oligonucleotides (22 to 59 bases in length) were synthesized chemically. 20 reassembly fragments were prepared by annealing of 20 forward and 20 reverse oligonucleotides. In one arm of the experiment, the nucleic acid building blocks (concentration 25 pmol/μl) were left untreated, and in another arm of the experiment the nucleic acid building blocks were subjected to the following MutS treatment protocol:

Mut-S treatment: Fragments (1000 pmol) were added to 349 μl of a reaction mix (20 mM Tris/Cl pH 8.0, 90 mM KCl, 1 mM DTT, 5 mM MgCl₂, 10% v/v glycerol) and supplemented with 17.9 μl MutS (Epicentre, 2 mg/ml). The reaction mixture was incubated for 1 hour at room temperature, transferred into Microcon YM-100 (Millipore) filtration units and spun for 20 min at 4,700 g. The flow through was loaded onto YM-10 (Millipore) filtration units and concentrated by centrifugation (30 min, 13,800 g). The retentate was recovered and the volume was adjusted to a final oligonucleotide concentration of approximately 25 pmol/μl.

The nucleic acid reassembly process of the invention was then continued using magnetic beads as solid support (the solid phase protocol used was according to Dynal A. S., Oslo, Norway, see Biomagnetic Techniques in Molecular Biology—Technical Handbook, 3rd edition, section 5.1 entitled: “Solid-phase gene assembly”, page 135-137), and using MutS-treated nucleic acid building blocks in one experimental arm and untreated nucleic acid building blocks in the other arm. The final nucleic acid reassembly product was made by step-wise cycles of assembly and washes to remove unbound fragment. The full-length product was removed from the beads by restriction digestion, amplified by PCR, cloned into a suitable vector and transformed into E. coli. To investigate the influence of the MutS treatment, 20 clones from each reassembly reaction arm were randomly picked, the respective plasmids isolated and the integrity of the inserted open reading frame checked by sequencing.

Results: Sequence comparison revealed that the MutS treatment increased the yield of correct open reading frames for the gene 1658 OT5 substantially.

### Example 5

Gene Reassembly

The following example describes manipulation of three related parental nucleotide sequences using gene reassembly. Each of the three related parental nucleotide sequence was aligned in the computer to determine demarcation points, and 17 such points were identified. Once each demarcation point was determined, the system determined the sequence of the 18 different fragments that would make up each parental gene. Each fragment from the parental sequence had a unique 5′ and 3′ overhang so only genes in the proper order could be reassembled by the computer. Because there were 18 fragments and three parents, the system had a total of 18×3=54 total fragments to analyze. It is advantageous for the system to pre-ligate each of the fragments in a process in order to store datafiles corresponding to every possible combination of pre-ligated fragments. This allows the system to determine the proper quantities of each pre-ligated fragment at each step in the ligation reaction in order to generate a resulting progeny population that has a predetermined PDF. Thus, in this example, the computer determined and stored the following pre-ligated sequences into its memory for EACH parent sequence. Accordingly, the following pre-ligation method is carried out on each parent sequence, the resulting data is stored to the computer.

The nomenclature “F1_{—}1” refers to the first fragment from the chosen parental sequence. The nomenclature “F1_{—}5” corresponds, as shown below, to a dataset comprising a combination of the first, second, third, fourth and fifth fragments of the chosen parental sequence. Thus, the following listing illustrates that the system can generate a dataset that stores every possible pre-ligated fragment for a given parent. This dataset is then used by the system to determine the proper quantities of each pre-ligated fragment to result in the desired final crossover population of progeny chimeric sequences.

Listing of Pre-Ligation Dataset for a Parent Sequence having 18 fragments.

F1_1 = F1_1
F1_2 = F1_1 + F2_2
F1_3 = F1_1 + F2_2 + F3_3
F1_4 = F1_1 + F2_2 + F3_3 + F4_4
F1_5 = F1_1 + F2_2 + F3_3 + F4_4 + F5_5
F1_6 = F1_1 + F2_2 + F3_3 + F4_4 + F5_5 + F6_6
F1_7 = F1_1 + F2_2 + F3_3 + F4_4 + F5_5 + F6_6 + F7_7
F1_8 = F1_1 + F2_2 + F3_3 + F4_4 + F5_5 + F6_6 + F7_7 + F8_8
F1_9 = F1_1 + F2_2 + F3_3 + F4_4 + F5_5 + F6_6 + F7_7 + F8_8 + F9_9
F1_10 = F1_1 + F2_2 + F3_3 + F4_4 + F5_5 + F6_6 + F7_7 + F8_8 + F9_9 + F10_10
F1_11 = F1_1 + F2_2 + F3_3 + F4_4 + F5_5 + F6_6 + F7_7 + F8_8 + F9_9 + F10_10 + F11_11
F1_12 = F1_1 + F2_2 + F3_3 + F4_4 + F5_5 + F6_6 + F7_7 + F8_8 + F9_9 + F10_10 + F11_11 + F12_12
F1_13 = F1_1 + F2_2 + F3_3 + F4_4 + F5_5 + F6_6 + F7_7 + F8_8 + F9_9 + F10_10 + F11_11 + F12_12 + F13_13
F1_14 = F1_1 + F2_2 + F3_3 + F4_4 + F5_5 + F6_6 + F7_7 + F8_8 + F9_9 + F10_10 + F11_11 + F12_12 + F13_13 +
F14_14
F1_15 = F1_1 + F2_2 + F3_3 + F4_4 + F5_5 + F6_6 + F7_7 + F8_8 + F9_9 + F10_10 + F11_11 + F12_12 + F13_13 +
F14_14 + F15_15
F1_16 = F1_1 + F2_2 + F3_3 + F4_4 + F5_5 + F6_6 + F7_7 + F8_8 + F9_9 + F10_10 + F11_11 + F12_12 + F13_13 +
F14_14 + F15_15 + F16_16
F1_17 = F1_1 + F2_2 + F3_3 + F4_4 + F5_5 + F6_6 + F7_7 + F8_8 + F9_9 + F10_10 + F11_11 + F12_12 + F13_13 +
F14_14 + F15_15 + F16_16 + F17_17
F1_18 = F1_1 + F2_2 + F3_3 + F4_4 + F5_5 + F6_6 + F7_7 + F8_8 + F9_9 + F10_10 + F11_11 + F12_12 + F13_13 +
F14_14 + F15_15 + F16_16 + F17_17 + F18_18
F2_2 = F2_2
F2_3 = F2_2 + F3_3
F2_4 = F2_2 + F3_3 + F4_4
F2_5 = F2_2 + F3_3 + F4_4 + F5_5
F2_6 = F2_2 + F3_3 + F4_4 + F5_5 + F6_6
F2_7 = F2_2 + F3_3 + F4_4 + F5_5 + F6_6 + F7_7
F2_8 = F2_2 + F3_3 + F4_4 + F5_5 + F6_6 + F7_7 + F8_8
F2_9 = F2_2 + F3_3 + F4_4 + F5_5 + F6_6 + F7_7 + F8_8 + F9_9
F2_10 = F2_2 + F3_3 + F4_4 + F5_5 + F6_6 + F7_7 + F8_8 + F9_9 + F10_10
F2_11 = F2_2 + F3_3 + F4_4 + F5_5 + F6_6 + F7_7 + F8_8 + F9_9 + F10_10 + F11_11
F2_12 = F2_2 + F3_3 + F4_4 + F5_5 + F6_6 + F7_7 + F8_8 + F9_9 + F10_10 + F11_11 + F12_12
F2_13 = F2_2 + F3_3 + F4_4 + F5_5 + F6_6 + F7_7 + F8_8 + F9_9 + F10_10 + F11_11 + F12_12 + F13_13
F2_14 = F2_2 + F3_3 + F4_4 + F5_5 + F6_6 + F7_7 + F8_8 + F9_9 + F10_10 + F11_11 + F12_12 + F13_13 + F14_14
F2_15 = F2_2 + F3_3 + F4_4 + F5_5 + F6_6 + F7_7 + F8_8 + F9_9 + F10_10 + F11_11 + F12_12 + F13_13 + F14_14 +
F15_15
F2_16 = F2_2 + F3_3 + F4_4 + F5_5 + F6_6 + F7_7 + F8_8 + F9_9 + F10_10 + F11_11 + F12_12 + F13_13 + F14_14 +
F15_15 + F16_16
F2_17 = F2_2 + F3_3 + F4_4 + F5_5 + F6_6 + F7_7 + F8_8 + F9_9 + F10_10 + F11_11 + F12_12 + F13_13 + F14_14 +
F15_15 + F16_16 + F17_17
F2_18 = F2_2 + F3_3 + F4_4 + F5_5 + F6_6 + F7_7 + F8_8 + F9_9 + F10_10 + F11_11 + F12_12 + F13_13 + F14_14 +
F15_15 + F16_16 + F17_17 + F18_18
F3_3 = F3_3
F3_4 = F3_3 + F4_4
F3_5 = F3_3 + F4_4 + F5_5
F3_6 = F3_3 + F4_4 + F5_5 + F6_6
F3_7 = F3_3 + F4_4 + F5_5 + F6_6 + F7_7
F3_8 = F3_3 + F4_4 + F5_5 + F6_6 + F7_7 + F8_8
F3_9 = F3_3 + F4_4 + F5_5 + F6_6 + F7_7 + F8_8 + F9_9
F3_10 = F3_3 + F4_4 + F5_5 + F6_6 + F7_7 + F8_8 + F9_9 + F10_10
F3_11 = F3_3 + F4_4 + F5_5 + F6_6 + F7_7 + F8_8 + F9_9 + F10_10 + F11_11
F3_12 = F3_3 + F4_4 + F5_5 + F6_6 + F7_7 + F8_8 + F9_9 + F10_10 + F11_11 + F12_12
F3_13 = F3_3 + F4_4 + F5_5 + F6_6 + F7_7 + F8_8 + F9_9 + F10_10 + F11_11 + F12_12 + F13_13
F3_14 = F3_3 + F4_4 + F5_5 + F6_6 + F7_7 + F8_8 + F9_9 + F10_10 + F11_11 + F12_12 + F13_13 + F14_14
F3_15 = F3_3 + F4_4 + F5_5 + F6_6 + F7_7 + F8_8 + F9_9 + F10_10 + F11_11 + F12_12 + F13_13 + F14_14 + F15_15
F3_16 = F3_3 + F4_4 + F5_5 + F6_6 + F7_7 + F8_8 + F9_9 + F10_10 + F11_11 + F12_12 + F13_13 + F14_14 + F15_15 +
F16_16
F3_17 = F3_3 + F4_4 + F5_5 + F6_6 + F7_7 + F8_8 + F9_9 + F10_10 + F11_11 + F12_12 + F13_13 + F14_14 + F15_15 +
F16_16 + F17_17
F3_18 = F3_3 + F4_4 + F5_5 + F6_6 + F7_7 + F8_8 + F9_9 + F10_10 + F11_11 + F12_12 + F13_13 + F14_14 + F15_15 +
F16_16 + F17_17 + F18_18
F4_4 = F4_4
F4_5 = F4_4 + F5_5
F4_6 = F4_4 + F5_5 + F6_6
F4_7 = F4_4 + F5_5 + F6_6 + F7_7
F4_8 = F4_4 + F5_5 + F6_6 + F7_7 + F8_8
F4_9 = F4_4 + F5_5 + F6_6 + F7_7 + F8_8 + F9_9
F4_10 = F4_4 + F5_5 + F6_6 + F7_7 + F8_8 + F9_9 + F10_10
F4_11 = F4_4 + F5_5 + F6_6 + F7_7 + F8_8 + F9_9 + F10_10 + F11_11
F4_12 = F4_4 + F5_5 + F6_6 + F7_7 + F8_8 + F9_9 + F10_10 + F11_11 + F12_12
F4_13 = F4_4 + F5_5 + F6_6 + F7_7 + F8_8 + F9_9 + F10_10 + F11_11 + F12_12 + F13_13
F4_14 = F4_4 + F5_5 + F6_6 + F7_7 + F8_8 + F9_9 + F10_10 + F11_11 + F12_12 + F13_13 + F14_14
F4_15 = F4_4 + F5_5 + F6_6 + F7_7 + F8_8 + F9_9 + F10_10 + F11_11 + F12_12 + F13_13 + F14_14 + F15_15
F4_16 = F4_4 + F5_5 + F6_6 + F7_7 + F8_8 + F9_9 + F10_10 + F11_11 + F12_12 + F13_13 + F14_14 + F15_15 +
F16_16
F4_17 = F4_4 + F5_5 + F6_6 + F7_7 + F8_8 + F9_9 + F10_10 + F11_11 + F12_12 + F13_13 + F14_14 + F15_15 +
F16_16 + F17_17
F4_18 = F4_4 + F5_5 + F6_6 + F7_7 + F8_8 + F9_9 + F10_10 + F11_11 + F12_12 + F13_13 + F14_14 + F15_15 +
F16_16 + F17_17 + F18_18
F5_5 = F5_5
F5_6 = F5_5 + F6_6
F5_7 = F5_5 + F6_6 + F7_7
F5_8 = F5_5 + F6_6 + F7_7 + F8_8
F5_9 = F5_5 + F6_6 + F7_7 + F8_8 + F9_9
F5_10 = F5_5 + F6_6 + F7_7 + F8_8 + F9_9 + F10_10
F5_11 = F5_5 + F6_6 + F7_7 + F8_8 + F9_9 + F10_10 + F11_11
F5_12 = F5_5 + F6_6 + F7_7 + F8_8 + F9_9 + F10_10 + F11_11 + F12_12
F5_13 = F5_5 + F6_6 + F7_7 + F8_8 + F9_9 + F10_10 + F11_11 + F12_12 + F13_13
F5_14 = F5_5 + F6_6 + F7_7 + F8_8 + F9_9 + F10_10 + F11_11 + F12_12 + F13_13 + F14_14
F5_15 = F5_5 + F6_6 + F7_7 + F8_8 + F9_9 + F10_10 + F11_11 + F12_12 + F13_13 + F14_14 + F15_15
F5_16 = F5_5 + F6_6 + F7_7 + F8_8 + F9_9 + F10_10 + F11_11 + F12_12 + F13_13 + F14_14 + F15_15 + F16_16
F5_17 = F5_5 + F6_6 + F7_7 + F8_8 + F9_9 + F10_10 + F11_11 + F12_12 + F13_13 + F14_14 + F15_15 + F16_16 +
F17_17
F5_18 = F5_5 + F6_6 + F7_7 + F8_8 + F9_9 + F10_10 + F11_11 + F12_12 + F13_13 + F14_14 + F15_15 + F16_16 +
F17_17 + F18_18
F6_6 = F6_6
F6_7 = F6_6 + F7_7
F6_8 = F6_6 + F7_7 + F8_8
F6_9 = F6_6 + F7_7 + F8_8 + F9_9
F6_10 = F6_6 + F7_7 + F8_8 + F9_9 + F10_10
F6_11 = F6_6 + F7_7 + F8_8 + F9_9 + F10_10 + F11_11
F6_12 = F6_6 + F7_7 + F8_8 + F9_9 + F10_10 + F11_11 + F12_12
F6_13 = F6_6 + F7_7 + F8_8 + F9_9 + F10_10 + F11_11 + F12_12 + F13_13
F6_14 = F6_6 + F7_7 + F8_8 + F9_9 + F10_10 + F11_11 + F12_12 + F13_13 + F14_14
F6_15 = F6_6 + F7_7 + F8_8 + F9_9 + F10_10 + F11_11 + F12_12 + F13_13 + F14_14 + F15_15
F6_16 = F6_6 + F7_7 + F8_8 + F9_9 + F10_10 + F11_11 + F12_12 + F13_13 + F14_14 + F15_15 + F16_16
F6_17 = F6_6 + F7_7 + F8_8 + F9_9 + F10_10 + F11_11 + F12_12 + F13_13 + F14_14 + F15_15 + F16_16 + F17_17
F6_18 = F6_6 + F7_7 + F8_8 + F9_9 + F10_10 + F11_11 + F12_12 + F13_13 + F14_14 + F15_15 + F16_16 + F17_17 +
F18_18
F7_7 = F7_7
F7_8 = F7_7 + F8_8
F7_9 = F7_7 + F8_8 + F9_9
F7_10 = F7_7 + F8_8 + F9_9 + F10_10
F7_11 = F7_7 + F8_8 + F9_9 + F10_10 + F11_11
F7_12 = F7_7 + F8_8 + F9_9 + F10_10 + F11_11 + F12_12
F7_13 = F7_7 + F8_8 + F9_9 + F10_10 + F11_11 + F12_12 + F13_13
F7_14 = F7_7 + F8_8 + F9_9 + F10_10 + F11_11 + F12_12 + F13_13 + F14_14
F7_15 = F7_7 + F8_8 + F9_9 + F10_10 + F11_11 + F12_12 + F13_13 + F14_14 + F15_15
F7_16 = F7_7 + F8_8 + F9_9 + F10_10 + F11_11 + F12_12 + F13_13 + F14_14 + F15_15 + F16_16
F7_17 = F7_7 + F8_8 + F9_9 + F10_10 + F11_11 + F12_12 + F13_13 + F14_14 + F15_15 + F16_16 + F17_17
F7_18 = F7_7 + F8_8 + F9_9 + F10_10 + F11_11 + F12_12 + F13_13 + F14_14 + F15_15 + F16_16 + F17_17 + F18_18
F8_8 = F8_8
F8_9 = F8_8 + F9_9
F8_10 = F8_8 + F9_9 + F10_10
F8_11 = F8_8 + F9_9 + F10_10 + F11_11
F8_12 = F8_8 + F9_9 + F10_10 + F11_11 + F12_12
F8_13 = F8_8 + F9_9 + F10_10 + F11_11 + F12_12 + F13_13
F8_14 = F8_8 + F9_9 + F10_10 + F11_11 + F12_12 + F13_13 + F14_14
F8_15 = F8_8 + F9_9 + F10_10 + F11_11 + F12_12 + F13_13 + F14_14 + F15_15
F8_16 = F8_8 + F9_9 + F10_10 + F11_11 + F12_12 + F13_13 + F14_14 + F15_15 + F16_16
F8_17 = F8_8 + F9_9 + F10_10 + F11_11 + F12_12 + F13_13 + F14_14 + F15_15 + F16_16 + F17_17
F8_18 = F8_8 + F9_9 + F10_10 + F11_11 + F12_12 + F13_13 + F14_14 + F15_15 + F16_16 + F17_17 + F18_18
F9_9 = F9_9
F9_10 = F9_9 + F10_10
F9_11 = F9_9 + F10_10 + F11_11
F9_12 = F9_9 + F10_10 + F11_11 + F12_12
F9_13 = F9_9 + F10_10 + F11_11 + F12_12 + F13_13
F9_14 = F9_9 + F10_10 + F11_11 + F12_12 + F13_13 + F14_14
F9_15 = F9_9 + F10_10 + F11_11 + F12_12 + F13_13 + F14_14 + F15_15
F9_16 = F9_9 + F10_10 + F11_11 + F12_12 + F13_13 + F14_14 + F15_15 + F16_16
F9_17 = F9_9 + F10_10 + F11_11 + F12_12 + F13_13 + F14_14 + F15_15 + F16_16 + F17_17
F9_18 = F9_9 + F10_10 + F11_11 + F12_12 + F13_13 + F14_14 + F15_15 + F16_16 + F17_17 + F18_18
F10_10 = F10_10
F10_11 = F10_10 + F11_11
F10_12 = F10_10 + F11_11 + F12_12
F10_13 = F10_10 + F11_11 + F12_12 + F13_13
F10_14 = F10_10 + F11_11 + F12_12 + F13_13 + F14_14
F10_15 = F10_10 + F11_11 + F12_12 + F13_13 + F14_14 + F15_15
F10_16 = F10_10 + F11_11 + F12_12 + F13_13 + F14_14 + F15_15 + F16_16
F10_17 = F10_10 + F11_11 + F12_12 + F13_13 + F14_14 + F15_15 + F16_16 + F17_17
F10_18 = F10_10 + F11_11 + F12_12 + F13_13 + F14_14 + F15_15 + F16_16 + F17_17 + F18_18
F11_11 = F11_11
F11_12 = F11_11 + F12_12
F11_13 = F11_11 + F12_12 + F13_13
F11_14 = F11_11 + F12_12 + F13_13 + F14_14
F11_15 = F11_11 + F12_12 + F13_13 + F14_14 + F15_15
F11_16 = F11_11 + F12_12 + F13_13 + F14_14 + F15_15 + F16_16
F11_17 = F11_11 + F12_12 + F13_13 + F14_14 + F15_15 + F16_16 + F17_17
F11_18 = F11_11 + F12_12 + F13_13 + F14_14 + F15_15 + F16_16 + F17_17 + F18_18
F12_12 = F12_12
F12_13 = F12_12 + F13_13
F12_14 = F12_12 + F13_13 + F14_14
F12_15 = F12_12 + F13_13 + F14_14 + F15_15
F12_16 = F12_12 + F13_13 + F14_14 + F15_15 + F16_16
F12_17 = F12_12 + F13_13 + F14_14 + F15_15 + F16_16 + F17_17
F12_18 = F12_12 + F13_13 + F14_14 + F15_15 + F16_16 + F17_17 + F18_18
F13_13 = F13_13
F13_14 = F13_13 + F14_14
F13_15 = F13_13 + F14_14 + F15_15
F13_16 = F13_13 + F14_14 + F15_15 + F16_16
F13_17 = F13_13 + F14_14 + F15_15 + F16_16 + F17_17
F13_18 = F13_13 + F14_14 + F15_15 + F16_16 + F17_17 + F18_18
F14_14 = F14_14
F14_15 = F14_14 + F15_15
F14_16 = F14_14 + F15_15 + F16_16
F14_17 = F14_14 + F15_15 + F16_16 + F17_17
F14_18 = F14_14 + F15_15 + F16_16 + F17_17 + F18_18
F15_15 = F15_15
F15_16 = F15_15 + F16_16
F15_17 = F15_15 + F16_16 + F17_17
F15_18 = F15_15 + F16_16 + F17_17 + F18_18
F16_16 = F16_16
F16_17 = F16_16 + F17_17
F16_18 = F16_16 + F17_17 + F18_18
F17_17 = F17_17
F17_18 = F17_17 + F18_18
F18_18 = F18_18

Once the sequence of each pre-ligated fragment is determined, the system begins to estimate the portions of each pre-ligated sequence to be used to generate the desired PDF. As discussed above, the ligation reaction for a sequence having 18 fragments preferably takes place as 18 separate reactions. Thus, the system generates a starting set of ligation reactions for each of the 18 separate ligations. It should be noted that each ligation step uses progressively fewer of the pre-ligated molecules. This is due to the fact that, for example, the third step of the ligation reaction would not require pre-ligated fragments starting with fragment 1 “F1” or fragment 2 (F2) since these fragments have already been ligated to other fragments by the third step in the ligation. At step three, there should only ligation of fragments that bind to the third fragment from each parent.

For example, the following are exemplary ligation reactions that take place within the memory of the computer system.

Number of Ligation Steps: 18

Simulated Ligation Volume of each Step (ul): 100

| Ligation Step | Ligation Step | Ligation Step | Ligation Step | Ligation Step |
|---|---|---|---|---|
| #1 | #2 | #3 | #4 | #5 |
| 0.6 ul of Fl_1 | 0.7 ul of F2_2 | 0.7 ul of F3_3 | 0.8 ul of F4_4 | 1.0 ul of F5_5 |
| 1.2 ul of F1_2 | 1.3 ul of F2_3 | 1.5 ul of F3_4 | 1.7 ul of F4_5 | 1.9 ul of F5_6 |
| 1.8 ul of F1_3 | 2.0 ul of F2_4 | 2.2 ul of F3_5 | 2.5 ul of F4_6 | 2.9 ul of F5_7 |
| 2.3 ul of F1_4 | 2.6 ul of F2_5 | 2.9 ul of F3_6 | 3.3 ul of F4_7 | 3.8 ul of F5_8 |
| 2.9 ul of F1_5 | 3.3 ul of F2_6 | 3.7 ul of F3_7 | 4.2 ul of F4_8 | 4.8 ul of F5_9 |
| 3.5 ul of F1_6 | 3.9 ul of F2_7 | 4.4 ul of F3_8 | 5.0 ul of F4_9 | 5.7 ul of F5_10 |
| 4.1 ul of F1_7 | 4.6 ul of F2_8 | 5.0 ul of F3_9 | 5.8 ul of F4_10 | 6.7 ul of F5_11 |
| 4.7 ul of F1_8 | 5.2 ul of F2_9 | 5.9 ul of F3_10 | 6.7 ul of F4_11 | 7.6 ul of F5_12 |
| 5.3 ul of F1_9 | 5.9 ul of F2_10 | 6.6 ul of F3_11 | 7.5 ul of F4_12 | 8.6 ul of F5_13 |
| 5.8 ul of F1_10 | 6.5 ul of F2_11 | 7.4 ul of F3_12 | 8.3 ul of F4_13 | 9.5 ul of F5_14 |
| 6.4 ul of F1_11 | 7.2 ul of F2_12 | 8.1 ul of F3_13 | 9.2 ul of F4_14 | 10.5 ul of F5_15 |
| 7.0 ul of F1_12 | 7.8 ul of F2_13 | 8.8 ul of F3_14 | 10.0 ul of F4_15 | 11.4 ul of F5_16 |
| 7.6 ul of F1_13 | 8.5 ul of F2_14 | 9.6 ul of F3_15 | 10.8 ul of F4_16 | 12.4 ul of F5_17 |
| 8.2 ul of F1_14 | 9.2 ul of F2_15 | 10.3 ul of F3_16 | 11.7 ul of F4_17 | 13.3 ul of F5_18 |
| 8.8 ul of F1_15 | 9.8 ul of F2_16 | 11.0 ul of F3_17 | 12.5 ul of F4_18 | |
| 9.4 ul of Fl_16 | 10.5 ul of F2_17 | 11.8 ul of F3_18 | | |
| 9.9 ul of F1_17 | 11.1 ul of F2_18 | | | |
| 10.5 ul of F1_18 | | | | |
| Ligation Step | Ligation Step | Ligation Step | Ligation Step | Ligation Step |
| #6: | #7 | #8 | #9 | #10 |
| 1.1 ul of F6_6 | 1.3 ul of F7_7 | 1.5 ul of F8_8 | 1.8 ul of F9_9 | 2.2 ul of F10_10 |
| 2.2 ul of F6_7 | 2.6 ul of F7_8 | 3.0 ul of F8_9 | 3.6 ul of F9_10 | 4.4 ul of F10_11 |
| 3.3 ul of F6_8 | 3.8 ul of F7_9 | 4.5 ul of F8_10 | 5.5 ul of F9_11 | 6.7 ul of F10_12 |
| 4.4 ul of F6_9 | 5.l ul of F7_10 | 6.l ul of F5_11 | 7.3 ul of F9_12 | 8.9 ul of F10_13 |
| 5.5 ul of F6_10 | 6.4 ul of F7_11 | 7.6 ul of F8_12 | 9.1 ul of F9_13 | 11.1 ul of F10_14 |
| 6.6 ul of F6_11 | 7.7 ul of F7_12 | 9.1 ul of F8_13 | 10.9 ul of F9_14 | 13.3 ul of F10_15 |
| 7.7 ul of F6_12 | 9.0 ul of F7_13 | 10.6 ul of F8_14 | 12.7 ul of F9_15 | 15.6 ul of F10_16 |
| 8.8 ul of F6_13 | 10.3 ul of F7_14 | 12.1 ul of F8_15 | 14.5 ul of F9_16 | 17.8 ul of F10_17 |
| 9.9 ul of F6_14 | 11.5 ul of F7_15 | 13.6 ul of F8_16 | 16.4 ul of F9_17 | 20.0 ul of F10_18 |
| 11.0 ul of F6_15 | 12.8 ul of F7_16 | 15.2 ul of F8_17 | 18.2 ul of F9_18 | |
| 12.1 ul of F6_16 | 14.1 ul of F7_17 | 16.7 ul of F8_18 | | |
| 13.2 ul of F6_17 | 15.4 ul of F7_18 | | | |
| 14.3 ul of F6_18 | | | | |
| Ligation Step | Ligation Step | Ligation Step | Ligation Step | Ligation Step |
| #11 | #12 | #13 | #14 | #15 |
| 2.8 ul of F11_11 | 3.6 ul of F12_12 | 4.8 ul of F13_13 | 6.7 ul of F14_14 | |
| 5.6 ul of F11_12 | 7.1 ul of F12_13 | 9.5 ul of F13_14 | 13.3 ul of F14_15 | 10.0 ul of F15_15 |
| 8.3 ul of F11_13 | 10.7 ul of F12_14 | 14.3 ul of F13_15 | 20.0 ul of F14_16 | 20.0 ul of F15_16 |
| 11.1 ul of F11_14 | 14.3 ul of F12_15 | 19.0 ul of F13_16 | 26.7 ul of F14_17 | 30.0 ul of F15_17 |
| 13.9 ul of F11_15 | 17.9 ul of F12_16 | 23.8 ul of F13_17 | 33.3 ul of F14_18 | 40.0 ul of F15_18 |
| 16.7 ul of F11_16 | 21.4 ul of F12_17 | 28.6 ul of F13_18 | | |
| 19.4 ul of F11_17 | 25.0 ul of F12_18 | | | |
| 22.2 ul of F11_18 | | | | |
| Ligation Step | Ligation Step | Ligation Step | | |
| #16 | #17 | #18 | | |
| 16.7 ul of F16_16 | 33.3 ul of F17_17 | 100.0 ul of F18_18 | | |
| 33.3 ul of F16_17 | 66.7 ul of F17_18 | | | |
| 50.0 ul of F16_18 | | | | |

Carrying out the preceding ligation reactions results in a calculated PDF. Thus, the system can then adjust the volumes of each pre-ligated fragment during a further round of simulated reassembly until the PDF matches the desired probability function. The majority of progeny molecules only have one or two crossover events. Adjusting the quantities of the ligation reactions, as shown below will skew the PDF so that it moves towards progeny molecules having more crossover events.

Computer Systems:

The methods of the invention, particular, the gene reassembly aspects of the invention, can use computer systems to carry out the methods described herein. In one aspect, the computer system is a conventional personal computer such as those based on an Intel microprocessor and running a Windows operating system. The output of the computer system is a fragment PDF that can be used as a recipe for producing reassembled progeny genes, and the estimated crossover PDF of those genes. The processing described herein can be performed by a personal computer using the MATLAB™ programming language and development environment. The invention is not limited to any particular hardware or software configuration. For example, computers based on other well-known microprocessors and running operating system software such as UNIX™, Linux, MacOS™ and others are contemplated.

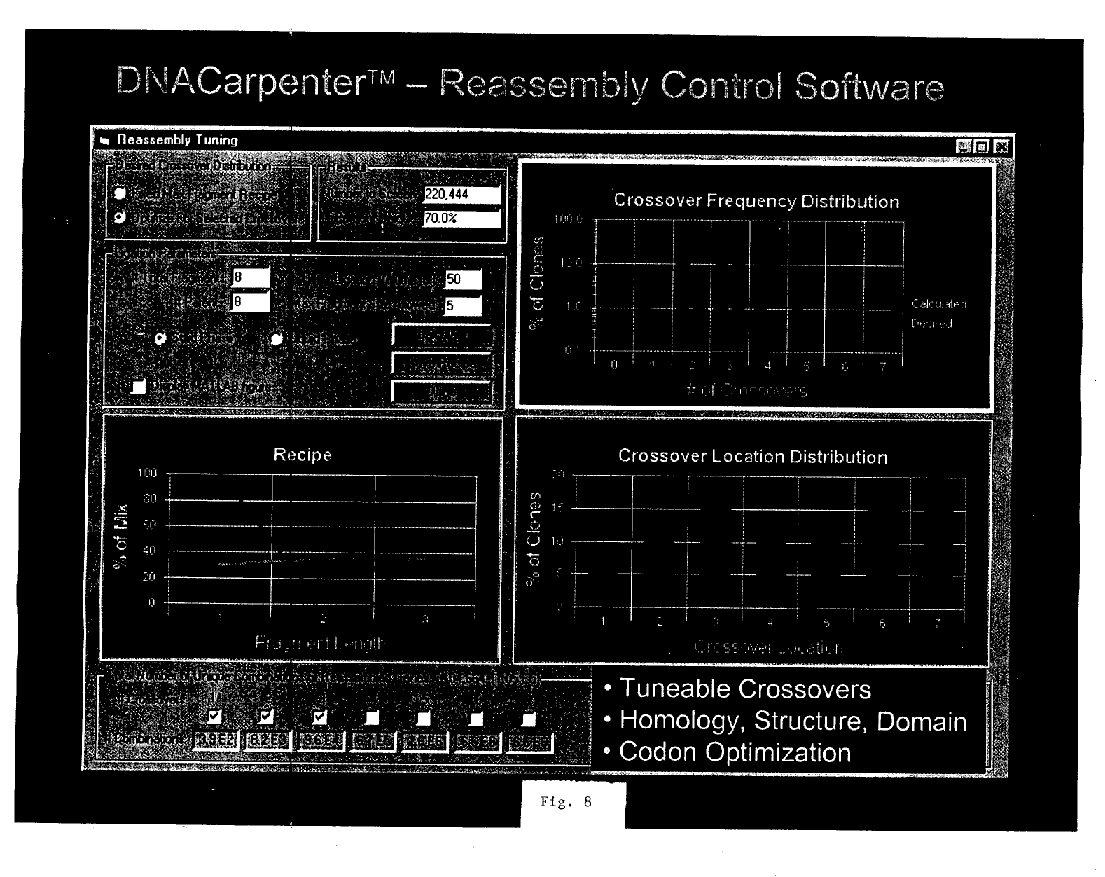

FIG. 8 illustrates an exemplary software program used in the methods of the invention. This “GENECARPENTER™” software program can be used as gene reassembly control software, and particularly in the methods of the invention for designing and making polynucleotides by iterative assembly of codon building blocks.

### Example 6

Iterative or Combinatorial Approach

In various aspects, this invention incorporates methods comprising introducing point mutations or codon mutations (e.g. by GSSM, where all possible amino acid substitutions are introduced at each position) followed by selection &/or screening, in combination with chimerization among selected products (e.g. positive hits) and/or parental sequences, and optionally repeating with one or more selection &/or screening step(s), and optionally one or more mutagenesis step(s). The screening or selection criteria according to this invention can include increases or decreases in one or more of the following: thermotolerance, ability to renature after denaturation by, e.g. heat (e.g. as determined with the helpd of a bomb calorimeter), storage life (e.g. shelf life at various temperatures), bioavailability, expression level, resistance to digestive tract destruction or to protease-mediated degradation, and activity &/or stability under different environmental conditions (e.g. exposure to different pH, pressure, salinity, solvent, etc. conditions).

Evolution by the GSSM™ method. The GSSM™ method was used to create a comprehensive library of point mutations in gene BD7746. A screen for thermotolerance was developed which measures the residual activity of an enzyme after heat challenge at high temperature. GSSM combined with a xylanase thermotolerance screen identified nine unique point mutants that had improved thermal tolerance. All nine mutations were combined in one gene using site-directed mutagenesis to generate a 9× mutant enzyme.

Generation of combinatorial GSSM™ variants using gene reassembly technology. To identify variants of the 9 point mutations with highest thermal tolerance and activity compared to the 9× variant, a Gene Reassembly library of all possible mutant combinations (2⁹) was constructed and screened. Using thermostability as the criterion, 33 unique combinations of the nine mutations were identified as up-mutants. A secondary screen was performed to select for variants with higher activity/expression than the evolved 933 . This screen yielded 10 variants with sequences possessing between 6 and 8 mutations in various combinations. All 10 variants have higher thermotolerance and improved activity over the 9× variant. These enzymes were subsequently purified and characterized.

Detailed Protocols:

Gene Site Saturation Mutagenesis and Activity Screening of BD7746. The BD7746 gene was amplified by PCR and cloned into the expression vector pTrcHis2 using the pTrcHis2 TOPO™ TA Cloning® Kit (Invitrogen, Carlsbad, Calif.). GSSM was performed as described previously (Short, JM 2001) using 64-fold degenerate oligonucleotides to randomize at each codon in the gene so that all possible amino acids would be encoded. The resultant GSSM library was transformed into XL1-Blue (Stratagene, La Jolla, Calif.) for screening.

Individual clones were arrayed in 96-well microtiter plates containing 200 μL of LB media and 100 μg/mL ampicillin using an automated colony picker (AutoGen, Ma). Four 96-well plates were screened per codon. The plates were incubated overnight at 37° C. These master plates were replicated using a 96-well pintool into fresh media containing antibiotic. The replica plates were sealed with a gas permeable adhesive film and incubated overnight at 37° C. After incubation, the seals were removed and the plates centrifuged at approximately 3000 g for 10 minutes. The supernatant was removed and the cells resuspended in 45 μL of 100 mM citrate/phosphate buffer (pH 6.0) containing 100 mM KCl (CP buffer). The plates were then covered with an adhesive aluminum seal and incubated at 80° C. for 20 minutes followed by the addition of 30 μL of 2% Azo-xylan prepared in CP buffer and incubation overnight at 37° C. After incubation, 200 μL of 100% ethanol was added and the plates were centrifuged at approximately 3000 g for 10 minutes. The supernatant was transferred to fresh plates and absorbance at 590 nm measured to quantify residual enzyme activity.

All nine mutations were combined in one gene using site-directed mutagenesis to generate a 9× mutant enzyme. The 9× gene, the wild-type gene and all nine single mutant genes were PCR amplified using primers designed to append an N-terminal hexahistidine tag. The PCR products were cloned into pTrcHis2 as described above.

GeneReassembly™ library construction and screening. The 591 bp XYL7746 gene (gene plus codons for hexahistidine tag) was divided into 5 segments according to the locations of the mutations in the GSSM clones. In this scenario, segments 1 and 3 corresponded to the wild-type gene while segments 2 and 4 contained 0-4 amino acid mutations each and segment 5 contained 0-1 mutations. Three of the segments, 1, 3 and 5 were produced by PCR where segments 1 and 3 used the wild-type template and segment 5 was made using two different templates (wild type and mutant S79P). Segments 2 and 4 were both made by annealing synthetic oligonucleotide containing 0-4 mutations each. After all the segments were made the library was constructed by first digesting the PCR products of segments 1, 3 and 5 to create overhangs compatible with those of the annealed oligomers 2 and 4. Segments 1-3 and 4-5 were ligated separately. The ligated 1-3 segment was amplified by PCR and the product was digested and ligated to segment 4-5. The final library (512 mutants; segments 1-5) was isolated and cloned into pTrcHis2 and transformed into XL1 Blue MRF' cells (Stratagene, La Jolla, Calif.) and was plated on solid LB medium containing 100 μg/mL ampicillin. Approximately 4000 colonies were auto-picked (see above) into approximately forty 96-well plates and were incubated at 37° C. overnight. The screening assay was performed as described above for the screening of the GSSM™ mutant library except that the resuspended cells were incubated for 60 minutes at 80° C. followed by addition of substrate and incubation of plates at 37° C. for 20 minutes.

A number of embodiments of the invention have been described. Nevertheless, it will be understood that various modifications may be made without departing from the spirit and scope of the invention. Accordingly, other embodiments are within the scope of the following claims.

What is claimed is:

1. A library of chimeric nucleic acids encoding a plurality of chimeric antigen binding polypeptides, the library made by a method comprising the following steps:
(a) providing a plurality of nucleic acids encoding a lambda light chain variable region polypeptide domain (V_{λ}) or a kappa light chain variable region polypeptide domain (V_{κ});
(b) providing a plurality of oligonucleotides encoding a J region polypeptide domain (V_{J});
(c) providing a plurality of nucleic acids encoding a lambda light chain constant region polypeptide domain (C_{λ}) or a kappa light chain constant region polypeptide domain (C_{κ});
(d) joining together a nucleic acid of step (a), a nucleic acid of step (c) and an oligonucleotide of step (b), wherein the oligonucleotide of step (b) is placed between the nucleic acids of step (a) and step (c) to generate a V-J-C chimeric nucleic acid coding sequence encoding a chimeric antigen binding polypeptide, and repeating this joining step to generate a library of chimeric nucleic acid coding sequences encoding a library of chimeric antigen binding polypeptides.

2. The library of claim 1, wherein an antigen binding polypeptide comprises an single chain antibody.

3. The library of claim 1, wherein an antigen binding polypeptide comprises a Fab fragment, an Fd fragment or an antigen binding complementarity determining region (CDR).

4. The library of claim 1, wherein the lambda light chain variable region polypeptide domain (Vλ) nucleic acid coding sequence or the kappa light chain variable region polypeptide domain (Vκ) nucleic acid coding sequence of step (a) are generated by an amplification reaction.

5. The library of claim 1, wherein lambda light chain constant region polypeptide domain (Cλ) nucleic acid coding sequence or the kappa light chain constant region polypeptide domain (Cκ) nucleic acid coding sequence of step (c) are generated by an amplification reaction.

6. The library of claim 4 or claim 5, wherein the amplification reaction comprises a polymerase chain reaction (PCR) amplification reaction using a pair of oligonucleotide primers.

7. The library of claim 6, wherein the oligonucleotide primers further comprise a restriction enzyme site.

8. The library of claim 1, wherein the lambda light chain variable region polypeptide domain (Vλ) nucleic acid coding sequence, the kappa light chain variable region polypeptide domain (Vκ) nucleic acid coding sequence, the lambda light chain constant region polypeptide domain (Cλ) nucleic acid coding sequence or the kappa light chain constant region polypeptide domain (Cκ) nucleic acid coding sequence is between about 99 and about 600 base pair residues in length.

9. The library of claim 8, wherein a nucleic acid coding sequence is between about 198 and about 402 base pair residues in length.

10. The library of claim 9, wherein a nucleic acid coding sequence is between about 300 and about 320 base pair residues in length.

11. The library of claim 4 or claim 5, wherein amplified nucleic acid is a mammalian nucleic acid.

12. The library of claim 11, wherein the amplified mammalian nucleic acid is a human nucleic acid.

13. The library of claim 4 or claim 5, wherein amplified nucleic acid is a genomic DNA, a cDNA or an RNA.

14. The library of claim 1, wherein an oligonucleotide encoding a J region polypeptide domain of step (b) is between about 9 and about 99 base pair residues in length.

15. The library of claim 14, wherein an oligonucleotide encoding a J region polypeptide domain of step (b) is between about 18 and about 81 base pair residues in length.

16. The library of claim 15, wherein an oligonucleotide encoding a J region polypeptide domain of step (b) is between about 36 and about 63 base pair residues in length.

17. The library of claim 1, wherein the joining of step (d) to generate a chimeric nucleic acid comprises a DNA ligase, a transcription or an amplification reaction.

18. The library of claim 17, wherein the amplification reaction comprises a polymerase chain reaction (PCR) amplification reaction.

19. The library of claim 18, wherein the amplification reaction comprises use of oligonucleotide primers.

20. The library of claim 19, wherein the oligonucleotide primers further comprises a restriction enzyme site.

21. The library of claim 17, wherein the transcription comprises a DNA polymerase transcription reaction.

22. A library of chimeric nucleic acids encoding a plurality of chimeric antigen binding polypeptides, the library made by a method comprising the following steps:
(a) providing a plurality of nucleic acids encoding an antibody heavy chain variable region polypeptide domain (V_{H});
(b) providing a plurality of oligonucleotides encoding a D region polypeptide domain (V_{D});
(c) providing a plurality of oligonucleotides encoding a J region polypeptide domain (V_{J});
(d) providing a plurality of nucleic acids encoding a heavy chain constant region polypeptide domain (C_{H});
(e) joining together a nucleic acid of step (a), a nucleic acid of step (d) and an oligonucleotide of step (b) and step (c), wherein the oligonucleotides of step (b) and step (c) are placed between the nucleic acids of step (a) and step (d) to generate a V-D-J-C chimeric nucleic acid coding sequence encoding a chimeric antigen binding polypeptide, and repeating this joining step to generate a library of chimeric nucleic acid coding sequences encoding a library of chimeric antigen binding polypeptides.

23. The library of claim 22, wherein an antigen binding polypeptide comprises an single chain antibody.

24. The library of claim 22, wherein an antigen binding polypeptide comprises a Fab fragment, an Fd fragment or an antigen binding complementarity determining region (CDR).

25. The library of claim 23 or claim 24, wherein an antigen binding polypeptide comprise a μ, γ, γ2, γ3, γ4, δ, ε, α1 or α2 constant region.

26. The library of claim 22, wherein the heavy chain variable region polypeptide domain (V_{H}) is generated by an amplification reaction.

27. The library of claim 22, wherein heavy chain constant region polypeptide domain (C_{H}) nucleic acid coding sequence is generated by an amplification reaction.

28. The library of claim 26 or claim 27, wherein the amplification reaction comprises a polymerase chain reaction (PCR) amplification reaction using a pair of oligonucleotide primers.

29. The library of claim 28, wherein the oligonucleotide primers further comprise a restriction enzyme site.

30. The library of claim 22, wherein the heavy chain variable region polypeptide domain (V_{H}) nucleic acid coding sequence or the heavy chain constant region polypeptide domain (C_{H}) nucleic acid coding sequence is between about 99 and about 600 base pair residues in length.

31. The library of claim 30, wherein a nucleic acid coding sequence is between about 198 and about 402 base pair residues in length.

32. The library of claim 31, wherein a nucleic acid coding sequence is between about 300 and about 320 base pair residues in length.

33. The library of claim 26 or claim 27, wherein amplified nucleic acid is a mammalian nucleic acid.

34. The library of claim 33, wherein the amplified mammalian nucleic acid is a human nucleic acid.

35. The library of claim 26 or claim 27, wherein amplified nucleic acid is a genomic DNA, a cDNA or an RNA.

36. The library of claim 22, wherein an oligonucleotide encoding a D region polypeptide domain of step (b) or a J region polypeptide domain of step (c) is between about 9 and about 99 base pair residues in length.

37. The library of claim 36, wherein the oligonucleotide is between about 18 and about 81 base pair residues in length.

38. The library of claim 37, wherein the oligonucleotide is between about 36 and about 63 base pair residues in length.

39. The library of claim 22, wherein the joining of step (e) to generate a chimeric nucleic acid comprises a DNA ligase, a transcription or an amplification reaction.

40. The library of claim 39, wherein the amplification reaction comprises a polymerase chain reaction (PCR) amplification reaction.

41. The library of claim 40, wherein the amplification reaction comprises use of oligonucleotide primers.

42. The library of claim 41, wherein the oligonucleotide primers further comprise a restriction enzyme site.

43. The library of claim 39, wherein the transcription comprises a DNA polymerase transcription reaction.

44. An expression vector comprising a chimeric nucleic acid selected from a library as set forth in claim 1 or claim 22.

45. A transformed cell comprising a chimeric nucleic acid selected from a library as set forth in claim 1 or claim 22.

46. A transformed cell comprising an expression vector as set forth in claim 44.

47. A non-human transgenic animal comprising a chimeric nucleic acid selected from a library as set forth in claim 1 or claim 22.

48. A method for making a chimeric antigen binding polypeptide comprising the following steps:
(a) providing a nucleic acid encoding a lambda light chain variable region polypeptide domain (V_{λ}) or a kappa light chain variable region polypeptide domain (V_{κ});
(b) providing an oligonucleotides encoding a J region polypeptide domain (V_{J});
(c) providing a nucleic acid encoding a lambda light chain constant region polypeptide domain (C_{λ}) or a kappa light chain constant region polypeptide domain (C_{κ});
(d) joining together a nucleic acid of step (a), a nucleic acid of step (c) and an oligonucleotide of step (b), wherein the oligonucleotide of step (b) is placed between the nucleic acids of step (a) and step (c) to generate a V-J-C chimeric nucleic acid coding sequence encoding a chimeric antigen binding polypeptide.

49. A method for making a library of chimeric antigen binding polypeptides comprising the following steps:
(a) providing a plurality of nucleic acids encoding a lambda light chain variable region polypeptide domain (V_{λ}) or a kappa light chain variable region polypeptide domain (V_{κ});
(b) providing a plurality of oligonucleotides encoding a J region polypeptide domain (Vⱼ);
(c) providing a plurality of nucleic acids encoding a lambda light chain constant region polypeptide domain (C_{λ}) or a kappa light chain constant region polypeptide domain (C_{κ});
(d) joining together a nucleic acid of step (a), a nucleic acid of step (c) and an oligonucleotide of step (b), wherein the oligonucleotide of step (b) is placed between the nucleic acids of step (a) and step (c) to generate a V-J-C chimeric nucleic acid coding sequence encoding a chimeric antigen binding polypeptide, and repeating this joining step to generate a library of chimeric nucleic acid coding sequences encoding a library of chimeric antigen binding polypeptides.

50. A method for making a chimeric antigen binding polypeptide comprising the following steps:
(a) providing a nucleic acid encoding an antibody heavy chain variable region polypeptide domain (V_{H});
(b) providing an oligonucleotide encoding a D region polypeptide domain (V_{D});
(c) providing an oligonucleotide encoding a J region polypeptide domain (V_{J});
(d) providing a nucleic acid encoding a heavy chain constant region polypeptide domain (C_{H});
(e) joining together a nucleic acid of step (a), a nucleic acid of step (d) and an oligonucleotide of step (b) and step (c), wherein the oligonucleotides of step (b) and step (c) are placed between the nucleic acids of step (a) and step (d) to generate a V-D-J-C chimeric nucleic acid coding sequence encoding a chimeric antigen binding polypeptide.

51. A method for making a library of chimeric antigen binding polypeptides comprising the following steps:
(a) providing a plurality of nucleic acids encoding an antibody heavy chain variable region polypeptide domain (V_{H});
(b) providing a plurality of oligonucleotides encoding a D region polypeptide domain (V_{D});
(c) providing a plurality of oligonucleotides encoding a J region polypeptide domain (V_{J});
(d) providing a plurality of nucleic acids encoding a heavy chain constant region polypeptide domain (C_{H});
(e) joining together a nucleic acid of step (a), a nucleic acid of step (d) and an oligonucleotide of step (b) and step (c), wherein the oligonucleotides of step (b) and step (c) are placed between the nucleic acids of step (a) and step (d) to generate a V-D-J-C chimeric nucleic acid coding sequence encoding a chimeric antigen binding polypeptide, and repeating this joining step to generate a library of chimeric nucleic acid coding sequences encoding a library of chimeric antigen binding polypeptides

52. The method of claim 48, 49, 50 or 51, further comprising screening the expressed chimeric antigen binding polypeptide for its ability to specifically bind an antigen.

53. The method of claim 48, 49, 50 or 51, further comprising mutagenizing the nucleic acid coding sequence encoding a chimeric antigen binding polypeptide by a method comprising an optimized directed evolution system or a synthetic ligation reassembly, or a combination thereof.

54. The method of claim 53, further comprising screening the mutagenized chimeric antigen binding polypeptide for its ability to specifically bind an antigen.

55. The method of claim 54, further comprising screening the mutagenized chimeric antigen binding polypeptide for its ability to specifically bind an antigen.

56. The method of claim 55, comprising identifying a mutagenized antigen binding site variant by its increased antigen binding affinity or antigen binding specificity as compared to the affinity or specificity of the chimeric antigen binding polypeptide before mutagenesis.

57. The method of claim 53, comprising screening the mutagenized chimeric antigen binding polypeptide for its ability to specifically bind an antigen by a method comprising phage display of the antigen binding site polypeptide.

58. The method of claim 53, comprising screening the mutagenized chimeric antigen binding polypeptide for its ability to specifically bind an antigen by a method comprising expression of the expressed antigen binding site polypeptide in a liquid phase.

59. The method of claim 53, comprising screening the mutagenized chimeric antigen binding polypeptide for its ability to specifically bind an antigen by a method comprising ribosome display of the antigen binding site polypeptide.

60. The method of claim 48, 49, 50 or 51, further comprising screening the chimeric antigen binding polypeptide for its ability to specifically bind an antigen by a method comprising immobilizing the polypeptide in a solid phase.

61. The method of claim 48, 49, 50 or 51, comprising screening the chimeric antigen binding polypeptide for its ability to specifically bind an antigen by a method comprising a capillary array.

62. The method of claim 48, 49, 50 or 51, comprising screening the chimeric antigen binding polypeptide for its ability to specifically bind an antigen by a method comprising a double-orificed container.

63. The method of claim 62, wherein the double-orificed container comprises a double-orificed capillary array.

64. The method of claim 63, wherein the double-orificed capillary array is a GIGAMATRIX™ capillary array.

65. A method for making a library of chimeric antigen binding polypeptides comprising the following steps:
(a) providing a plurality of V-J-C chimeric nucleic acids encoding a chimeric antigen binding polypeptide made by a method as set forth in claim 48 or a plurality of V-D-J-C chimeric nucleic acids encoding a chimeric antigen binding polypeptide made by a method as set forth in claim 50;
(b) providing a plurality of oligonucleotides, wherein each oligonucleotide comprises a sequence homologous to a chimeric nucleic acid of step (a), thereby targeting a specific sequence of the chimeric nucleic acid, and a sequence that is a variant of the chimeric nucleic acid; and
(c) generating “n” number of progeny polynucleotides comprising non-stochastic sequence variations by replicating the chimeric nucleic acid of step (a) with the oligonucleotides of step (b), wherein n is an integer, thereby generating a library of chimeric antigen binding polypeptides.

66. The method of claim 65, wherein the sequence homologous to the chimeric nucleic acid is x bases long, wherein x is an integer between 3 and 100.

67. The method of claim 66, wherein the sequence homologous to the chimeric nucleic acid is x bases long, wherein x is an integer between 5 and 50.

68. The method of claim 67, wherein the sequence homologous to the chimeric nucleic acid is x bases long, wherein x is an integer between 10 and 30.

69. The method of claim 65, wherein the sequence that is a variant of the chimeric nucleic acid is x bases long, wherein x is an integer between 1 and 50.

70. The method of claim 69, wherein the sequence that is a variant of the chimeric nucleic acid is x bases long, wherein x is an integer between 2 and 20.

71. The method of claim 65, wherein the oligonucleotide of step (b) further comprises a second sequence homologous to the chimeric nucleic acid and the variant sequence is flanked by the sequences homologous to the chimeric nucleic acid.

72. The method of claim 71, wherein the second sequence that is a variant of the chimeric nucleic acid is x bases long, wherein x is an integer between 1 and 50.

73. The method of claim 72, wherein the second sequence is x bases long, wherein x is 3, 6, 9or 12.

74. The method of claim 65, wherein the oligonucleotides comprise variant sequences targeting a chimeric nucleic acid codon, thereby generating a plurality of progeny chimeric polynucleotides comprising a plurality of variant codons.

75. The method of claim 74, wherein the variant sequences generate variant codons encoding all nineteen naturally-occurring amino acid variants for a targeted codon, thereby generating all nineteen possible natural amino acid variations at the residue encoded by the targeted codon.

76. The method of claim 75, wherein the oligonucleotides comprise variant sequences targeting a plurality of chimeric nucleic acid codons.

77. The method of claim 76, wherein the oligonucleotides comprising variant sequences target all of the codons in the chimeric nucleic acid, thereby generating a plurality of progeny polypeptides wherein all amino acids are non-stochastic variants of the polypeptide encoded by the chimeric nucleic acid.

78. The method of claim 77, wherein the variant sequences generate variant codons encoding all nineteen naturally-occurring amino acid variants for all of the chimeric nucleic acid codons, thereby generating a plurality of progeny polypeptides wherein all amino acids are non-stochastic variants of the polypeptide encoded by the chimeric nucleic acid and a variant for all nineteen possible natural amino acids at all of the codons.

79. The method of claim 65, wherein the n is an integer between 1 and about 10³⁰.

80. The method of claim 79, wherein the n is an integer between about 10² and about 10².

81. The method of claim 80, wherein the n is an integer between about 10² and about 10¹⁰.

82. The method of claim 65, wherein the replicating of step (c) comprises an enzyme-based replication.

83. The method of claim 82, wherein the enzyme-based replication comprises a polymerase-based amplification reaction.

84. The method of claim 83, wherein the amplification reaction comprises a polymerase chain reaction (PCR).

85. The method of claim 82, wherein the enzyme-based replication comprises an error-free polymerase reaction.

86. The method of claim 65, wherein an oligonucleotide of step (b) further comprises a nucleic acid sequence capable of introducing one or more nucleotide residues into the template polynucleotide.

87. The method of claim 86, wherein an oligonucleotide of step (b) further comprises a nucleic acid sequence capable of deleting one or more residue from the template polynucleotide.

88. The method of claim 87, wherein the oligonucleotide of step (b) further comprises addition of one or more stop codons to the template polynucleotide.

89. A method for making a library of chimeric antigen binding polypeptides comprising the following steps:
(a) providing x number of V-J-C chimeric nucleic acids encoding a chimeric antigen binding polypeptide made by a method as set forth in claim 48 or x number of V-D-J-C chimeric nucleic acids encoding a chimeric antigen binding polypeptide made by a method as set forth in claim 50;
(b) providing y number of building block polynucleotides, wherein y is an integer, and the building block polynucleotides are designed to cross-over reassemble with a chimeric nucleic acid of step (a) at predetermined sequences and comprise a sequence that is a variant of the chimeric nucleic acid and a sequence homologous to the chimeric nucleic acid flanking the variant sequence; and,
(c) combining at least one building block polynucleotide with at least one chimeric nucleic acid such that the building block polynucleotide cross-over reassembles with the chimeric nucleic acid to generate non-stochastic progeny chimeric polynucleotides, thereby generating a library of polynucleotides encoding chimeric antigen binding polypeptides.

90. The method of claim 89, wherein x is an integer between 1 and about 10¹⁰.

91. The method of claim 90, wherein the x is an integer between about 10 and about 10².

92. The method of claim 1, wherein the x is an integer selected from the group consisting of 1, 2, 3, 4, 5, 6, 7, 8, 9 and 10.

93. The method of claim 89, wherein a plurality of building block polynucleotides are used and the variant sequences target a chimeric nucleic acid codon to generate a plurality of progeny polynucleotides that are variants of the targeted codon, thereby generating a plurality of natural amino acid variations at a residue in a polypeptide encoded by the chimeric nucleic acid.

94. The method of claim 93, wherein the variant sequences generate variant codons encoding all nineteen naturally-occurring amino acid variants for the targeted codon, thereby generating all nineteen possible natural amino acid variations at the residue encoded by the targeted codon in a polypeptide encoded by the chimeric nucleic acid.

95. The method of claim 94, wherein a plurality of building block polynucleotides are used, and the variant sequences target a plurality of chimeric nucleic acid codons, thereby generating a plurality of codons that are variants of the targeted codons and a plurality of natural amino acid variations at a plurality of residues encoded by the targeted codon in a polypeptide encoded by the chimeric nucleic acid.

96. The method of claim 95, wherein the variant sequences generate variant codons in all of the codons in the chimeric nucleic acid, thereby generating a plurality of progeny polypeptides wherein all amino acids are non-stochastic variants of the polypeptide encoded by the chimeric nucleic acid.

97. The method of claim 96, wherein the variant sequences generate variant codons encoding all nineteen naturally-occurring amino acid variants for all of the chimeric nucleic acid codons, thereby generating a plurality of progeny polypeptides wherein all amino acids are non-stochastic variants of the polypeptide encoded by the chimeric nucleic acid and a variant for all nineteen possible natural amino acids at all of the codons.

98. The method of claim 93, wherein all of the codons in an antigen binding site are targeted.

99. The method of claim 89, wherein the library comprises between 1 and about 10³⁰ members.

100. The method of claim 99, wherein the library comprises between about 10² and about 10²⁰ members.

101. The method of claim 100, wherein the library comprises between about 10³ and about 10¹⁰ members.

102. The method of claim 89, wherein an end of a building block polynucleotide comprises at least about 6 nucleotides homologous to a chimeric nucleic acid.

103. The method of claim 102, wherein an end of a building block polynucleotide comprises at least about 15 nucleotides homologous to a chimeric nucleic acid.

104. The method of claim 103, wherein an end of a building block polynucleotide comprises at least about 21 nucleotides homologous to a chimeric nucleic acid.

105. The method of claim 89, wherein combining one or more building block polynucleotides with a chimeric nucleic acid comprises z cross-over events between the building block polynucleotides and the chimeric nucleic acid, wherein y is an integer between 1 and about 10²⁰.

106. The method of claim 105, wherein z is an integer between about 10 and about 10¹⁰.

107. The method of claim 106, wherein z is an integer between about 10² and about 10⁵.

108. The method of claim 89, wherein a non-stochastic progeny chimeric polynucleotide differs from a chimeric nucleic acid in z number of residues, wherein z is between 1 and about 10⁴.

109. The method of claim 108, wherein a non-stochastic progeny chimeric polynucleotide differs from the template polynucleotide in z number of residues, wherein z is between 10 and about 10³.

110. The method of claim 109, wherein a non-stochastic progeny chimeric polynucleotide differs from the template polynucleotide in z number of residues, wherein z is selected from the group consisting of 1, 2, 3, 4, 5, 6, 7, 8, 9 and 10.

111. The method of claim 89, wherein a non-stochastic progeny chimeric polynucleotide differs from a chimeric nucleic acid in z number of codons, wherein z is between 1 and about 10⁴.

112. The method of claim 111, wherein a non-stochastic progeny chimeric polynucleotide differs from a chimeric nucleic acid in z number of codons, wherein z is between 10 and about 10³.

113. The method of claim 112, wherein a non-stochastic progeny chimeric polynucleotide differs from a chimeric nucleic acid in z number of codons, wherein z is selected from the group consisting of 1, 2, 3, 4, 5, 6, 7, 8, 9 and 10.

114. The method of claim 48, 49, 50 or 51, further comprising mutagenizing the nucleic acid encoding the chimeric antigen binding polypeptide.

115. The method of claim 114, wherein the nucleic is mutagenized by a method comprising an optimized directed evolution system or a synthetic ligation reassembly, or a combination thereof.

116. The method of claim 114, wherein the nucleic is mutagenized by a method comprising gene site saturated mutagenesis (GSSM), step-wise nucleic acid reassembly, error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, synthetic ligation reassembly (SLR) or a combination thereof.

117. The method of claim 114, wherein the nucleic is mutagenized by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation or a combination thereof.
